SEQUENCE LISTING (58 sequences)

SEQ ID NO: 1 - protein, 915 aa, Homo sapiens
MARRSSFQSCQIISLFTFAVGVSICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILRWLTLPDHERPSVYAFYSEQPDFSGHKYGPFGPEESSYGSPFTPAKRPKRKVAPKRRQERPVAPPKKRRRKIHRMDHYAAETRQDKMTNPLREIDKIVGQLMDGLKQLKLRRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGPTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMLLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 2 - DNA, 3231 bp, Homo sapiens
cgtgaaggcaaagagaacacgctgcaaaaggcttccaagaatcctcgacatggcaaggaggagctcgttccagtcgtgtcagataatatccctgttcacttttgccgttggagtcagtatctgcttaggattcactgcacatcgaattaagagagcagaaggatgggaggaaggtcctcctacagtgctatcagactccccctggaccaacatctccggatcttgcaagggcaggtgctttgaacttcaagaggctggacctcctgattgtcgctgtgacaacttgtgtaagagctataccagttgctgccatgactttgatgagctgtgtttgaagacagcccgtggctgggagtgtactaaggacagatgtggagaagtcagaaatgaagaaaatgcctgtcactgctcagaggactgcttggccaggggagactgctgtaccaattaccaagtggtttgcaaaggagagtcgcattgggttgatgatgactgtgaggaaataaaggccgcagaatgccctgcagggtttgttcgccctccattaatcatcttctccgtggatggcttccgtgcatcatacatgaagaaaggcagcaaagtcatgcctaatattgaaaaactaaggtcttgtggcacacactctccctacatgaggccggtgtacccaactaaaacctttcctaacttatacactttggccactgggctatatccagaatcacatggaattgttggcaattcaatgtatgatcctgtatttgatgccacttttcatctgcgagggcgagagaaatttaatcatagatggtggggaggtcaaccgctatggattacagccaccaagcaaggggtgaaagctggaacattcttttggtctgttgtcatccctcacgagcggagaatattaaccatattgcggtggctcaccctgccagatcatgagaggccttcggtctatgccttctattctgagcaacctgatttctctggacacaaatatggccctttcggccctgaggagagtagttatggctcaccttttactccggctaagagacctaagaggaaagttgcccctaagaggagacaggaaagaccagttgctcctccaaagaaaagaagaagaaaaatacataggatggatcattatgctgcggaaactcgtcaggacaaaatgacaaatcctctgagggaaatcgacaaaattgtggggcaattaatggatggactgaaacaactaaaactgcgtcggtgtgtcaacgtcatctttgtcggagaccatggaatggaagatgtcacatgtgatagaactgagttcttgagtaattacctaactaatgtggatgatattactttagtgcctggaactctaggaagaattcgatccaaatttagcaacaatgctaaatatgaccccaaagccattattgccaatctcacgtgtaaaaaaccagatcagcactttaagccttacttgaaacagcaccttcccaaacgtttgcactatgccaacaacagaagaattgaggatatccatttattggtggaacgcagatggcatgttgcaaggaaacctttggatgtttataagaaaccatcaggaaaatgctttttccagggagaccacggatttgataacaaggtcaacagcatgcagactgtttttgtaggttatggcccaacatttaagtacaagactaaagtgcctccatttgaaaacattgaactttacaatgttatgtgtgatctcctgggattgaagccagctcctaataatgggacccatggaagtttgaatcatctcctgcgcactaataccttcaggccaaccatgccagaggaagttaccagacccaattatccagggattatgtaccttcagtctgattttgacctgggctgcacttgtgatgataaggtagagccaaagaacaagttggatgaactcaacaaacggcttcatacaaaagggtctacagaagagagacacctcctctatgggcgacctgcagtgctttatcggactagatatgatatcttatatcacactgactttgaaagtggttatagtgaaatattcctaatgctactctggacatcatatactgtttccaaacaggctgaggtttccagcgttcctgaccatctgaccagttgcgtccggcctgatgtccgtgtttctccgagtttcagtcagaactgtttggcctacaaaaatgataagcagatgtcctacggattcctctttcctccttatctgagctcttcaccagaggctaaatatgatgcattccttgtaaccaatatggttccaatgtatcctgctttcaaacgggtctggaattatttccaaagggtattggtgaagaaatatgcttcggaaagaaatggagttaacgtgataagtggaccaatcttcgactatgactatgatggcttacatgacacagaagacaaaataaaacagtacgtggaaggcagttccattcctgttccaactcactactacagcatcatcaccagctgtctggatttcactcagcctgccgacaagtgtgacggccctctctctgtgtcctccttcatcctgcctcaccggcctgacaacgaggagagctgcaatagctcagaggacgaatcaaaatgggtagaagaactcatgaagatgcacacagctagggtgcgtgacattgaacatctcaccagcctggacttcttccgaaagaccagccgcagctacccagaaatcctgacactcaagacatacctgcatacatatgagagcgagatttaactttctgagcatctgcagtacagtcttatcaactggttgtatatttttatattgtttttgtatttattaatttgaaaccaggacattaaaaatgttagtattttaatcctgtaccaaatctgacatattatgcctgaatgactccactgtttttctctaatgcttgatttaggtagccttgtgttctgagtagagcttgtaataaatactgcagcttgagaaaaagtggaagcttctaaatggtgctgcagatttgatatttgcattgaggaaatattaattttccaatgcacagttgccacatttagtcctgtactgtatggaaacactgattttgtaaagttgcctttatttgctgttaactgttaactatgacagatatatttaagccttataaaccaatcttaaacataataaatcacacattcagtttt

SEQ ID NO: 3 - protein, 364 aa, Homo sapiens
MAAISTSIPVISQPQFTAMNEPQCFYNESIAFFYNRSGKHLATEWNTVSKLVMGLGITVCIFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIENCSNMAPLYSDSYLVFWAIFNLVTFVVMVVLYAHIFGYVRQRTMRMSRHSSGPRRNRDTMMSLLKTVVIVLGAFIICWTPGLVLLLLDVCCPQCDVLAYEKFFLLLAEFNSAMNPIIYSYRDKEMSATFRQILCCQRSENPTGPTEGSDRSASSLNHTILAGVHSNDHSVV

SEQ ID NO: 4 - protein, 351 aa, Homo sapiens
MVIMGQCYYNETIGFFYNNSGKELSSHWRPKDVVVVALGLTVSVLVLLTNLLVIAAIASNRRFHQPIYYLLGNLAAADLFAGVAYLFLMFHTGPRTARLSLEGWFLRQGLLDTSLTASVATLLAIAVERHRSVMAVQLHSRLPRGRVVMLIVGVWVAALGLGLLPAHSWHCLCALDRCSRMAPLLSRSYLAVWALSSLLVFLLMVAVYTRIFFYVRRRVQRMAEHVSCHPRYRETTLSLVKTVVIILGAFVVCWTPGQVVLLLDGLGCESCNVLAVEKYFLLLAEANSLVNAAVYSCRDAEMRRTFRRLLCCACLRQSTRESVHYTSSAQGGASTRIMLPENGHPLMDSTL

SEQ ID NO: 5 - protein, 353 aa, Homo sapiens
MNECHYDKHMDFFYNRSNTDTVDDWTGTKLVIVLCVGTFFCLFIFFSNSLVIAAVIKNRKFHFPFYYLLANLAAADFFAGIAYVFLMFNTGPVSKTLTVNRWFLRQGLLDSSLTASLTNLLVIAVERHMSIMRMRVHSNLTKKRVTLLILLVWAIAIFMGAVPTLGWNCLCNISACSSLAPIYSRSYLVFWTVSNLMAFLIMVVVYLRIYVYVKRKTNVLSPHTSGSISRRRTPMKLMKTVMTVLGAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQGAVCNKSTS

SEQ ID NO: 6 - protein, 370 aa, Homo sapiens
MGDRRFIDFQFQDSNSSLRPRLGNATANNTCIVDDSFKYNLNGAVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 7 - protein, 372 aa, Homo sapiens [sequence omitted]

SEQ ID NO: 8 - protein, 344 aa, Homo sapiens
MVSVNSSHCFYNDSFKYTLYGCMFSMVFVLGLISNCVAIYIFICVLKVRNETTTYMINLAMSDLLFVFTLPFRIFYFTTRNWPFGDLLCKISVMLFYTNMYGSILFLTCISVDRFLAIVYPFKSKTLRTKRNAKIVCTGVWLTVIGGSAPAVFVQSTHSQGNNASEACFENFPEATWKTYLSRIVIFIEIVGFFIPLILNVTCSSMVLKTLTKPVTLSRSKINKTKVLKMIFVHLIIFCFCFVPYNINLILYSLVRTQTFVNCSVVAAVRTMYPITLCIAVSNCCFDPIVYYFTSDTIQNSIKMKNWSVRRSDFRFSEVHGAENFIQHNLQTLKSKIFDNESAA

SEQ ID NO: 9 - protein, 1116 aa, Homo sapiens
MERAISPGLLVRALLLLLLLLGLAARTVAAGRARGLPAPTAEAAFGLGAAAAPTSATRVPAAGAVAAAEVTVEDAEALPAAAGEQEPRGPEPDDETELRPRGRSLVIISTLDGRIAALDPENHGKKQWDLDVGSGSLVSSSLSKPEVFGNKMIIPSLDGALFQWDQDRESMETVPFTVESLLESSYKFGDDVVLVGGKSLTTYGLSAYSGKVRYICSALGCRQWDSDEMEQEEDILLLQRTQKTVRAVGPRSGNEKWNFSVGHFELRYIPDMETRAGFIESTFKPNENTEESKIISDVEEQEAAIMDIVIKVSVADWKVMAFSKKGGHLEWEYQFCTPIASAWLLKDGKVIPISLFDDTSYTSNDDVLEDEEDIVEAARGATENSVYLGMYRGQLYLQSSVRISEKFPSSPKALESVTNENAIIPLPTIKWKPLIHSPSRTPVLVGSDEFDKCLSNDKFSHEEYSNGALSILQYPYDNGYYLPYYKRERNKRSTQITVRFLDNPHYNKNIRKKDPVLLLHWWKEIVATILFCIIATTFIVRRLFHPHPHRQRKESETQCQTENKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFATKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 10 - DNA, 4659 bp, Homo sapiens
ggaaagtccaccttccccaacaaggccagcctgggaacatggagtggcagcggccgcagccaatgagagagcaaacgcgcggaaagtttgctcaatgggcgatgtccgagataggctgtcactcaggtggcagcggcagaggccgggctgagacgtggccaggggaacacggctggctgtccaggccgtcggggcggcagtagggtccctagcacgtccttgccttcttgggagctccaagcggcgggagaggcaggcgtcagtggctgcgcctccatgcctgcgcgcggggcgggacgctgatggagcgcgccatcagcccggggctgctggtacgggcgctgctgctgctgctgctgctgctggggctcgcggcaaggacggtggccgcggggcgcgcccgtggcctcccagcgccgacggcggaggcggcgttcggcctcggggcggccgctgctcccacctcagcgacgcgagtaccggcggcgggcgccgtggctgcggccgaggtgactgtggaggacgctgaggcgctgccggcagccgcgggagagcaggagcctcggggtccggaaccagacgatgagacagagttgcgaccgcgcggcaggtcattagtaattatcagcactttagatgggagaattgctgccttggatcctgaaaatcatggtaaaaagcagtgggatttggatgtgggatccggttccttggtgtcatccagccttagcaaaccagaggtatttgggaataagatgatcattccttccctggatggagccctcttccagtgggaccaagaccgtgaaagcatggaaacagttcctttcacagttgaatcacttcttgaatcttcttataaatttggagatgatgttgttttggttggaggaaaatctctgactacatatggactcagtgcatatagtggaaaggtgaggtatatctgttcagctctgggttgtcgccaatgggatagtgacgaaatggaacaagaggaagacatcctgcttctacagcgtacccaaaaaactgttagagctgtcggacctcgcagtggcaatgagaagtggaatttcagtgttggccactttgaacttcggtatattccagacatggaaacgagagccggatttattgaaagcacctttaagcccaatgagaacacagaagagtctaaaattatttcagatgtggaagaacaggaagctgccataatggacatagtgataaaggtttcggttgctgactggaaagttatggcattcagtaagaagggaggacatctggaatgggagtaccagttttgtactccaattgcatctgcctggttacttaaggatgggaaagtcattcccatcagtctttttgatgatacaagttatacatctaatgatgatgttttagaagatgaagaagacattgtagaagctgccagaggagccacagaaaacagtgtttacttgggaatgtatagaggccagctgtatctgcagtcatcagtcagaatttcagaaaagtttccttcaagtcccaaggctttggaatctgtcactaatgaaaacgcaattattcctttaccaacaatcaaatggaaacccttaattcattctccttccagaactcctgtcttggtaggatctgatgaatttgacaaatgtctcagtaatgataagttttctcatgaagaatatagtaatggtgcactttcaatcttgcagtatccatatgataatggttattatctaccatactacaagagggagaggaacaaacgaagcacacagattacagtcagattcctcgacaacccacattacaacaagaatatccgcaaaaaggatcctgttcttcttttacactggtggaaagaaatagttgcaacgattttgttttgtatcatagcaacaacgtttattgtgcgcaggcttttccatcctcatcctcacaggcaaaggaaggagtctgaaactcagtgtcaaactgaaaataaatatgattctgtaagtggtgaagccaatgacagtagctggaatgacataaaaaactctggatatatatcacgatatctaactgattttgagccaattcaatgcctgggacgtggtggctttggagttgtttttgaagctaaaaacaaagtagatgactgcaattatgctatcaagaggatccgtctccccaatagggaattggctcgggaaaaggtaatgcgagaagttaaagccttagccaagcttgaacacccgggcattgttagatatttcaatgcctggctcgaagcaccaccagagaagtggcaagaaaagatggatgaaatttggctgaaagatgaaagcacagactggccactcagctctcctagcccaatggatgcaccatcagttaaaatacgcagaatggatcctttcgctacaaaagaacatattgaaatcatagctccttcaccacaaagaagcaggtctttttcagtagggatttcctgtgaccagacaagttcatctgagagccagttctcaccactggaattctcaggaatggaccatgaggacatcagtgagtcagtggatgcagcatacaacctccaggacagttgccttacagactgtgatgtggaagatgggactatggatggcaatgatgaggggcactcctttgaactttgtccttctgaagcttctccttatgtaaggtcaagggagagaacctcctcttcaatagtatttgaagattctggctgtgataatgcttccagtaaagaagagccgaaaactaatcgattgcatattggcaaccattgtgctaataaactaactgctttcaagcccaccagtagcaaatcttcttctgaagctacattgtctatttctcctccaagaccaaccactttaagtttagatctcactaaaaacaccacagaaaaactccagcccagttcaccaaaggtgtatctttacattcaaatgcagctgtgcagaaaagaaaacctcaaagactggatgaatggacgatgtaccatagaggagagagagaggagcgtgtgtctgcacatcttcctgcagatcgcagaggcagtggagtttcttcacagtaaaggactgatgcacagggacctcaagccatccaacatattctttacaatggatgatgtggtcaaggttggagactttgggttagtgactgcaatggaccaggatgaggaagagcagacggttctgaccccaatgccagcttatgccagacacacaggacaagtagggaccaaactgtatatgagcccagagcagattcatggaaacagctattctcataaagtggacatcttttctttaggcctgattctatttgaattgctgtatccattcagcactcagatggagagagtcaggaccttaactgatgtaagaaatctcaaatttccaccattatttactcagaaatatccttgtgagtacgtgatggttcaagacatgctctctccatcccccatggaacgacctgaagctataaacatcattgaaaatgctgtatttgaggacttggactttccaggaaaaacagtgctcagacagaggtctcgctccttgagttcatcgggaacaaaacattcaagacagtccaacaactcccatagccctttgccaagcaattagccttaagttgtgctagcaaccctaataggtgatgcagataatagcctacttcttagaatatgcctgtccaaaattgcagacttgaaaagtttgttcttcgctcaatttttttgtggactactttttttatatcaaatttaagctggatttgggggcataacctaatttgagccaactcctgagttttgctatacttaaggaaagggctatctttgttctttgttagtctcttgaaactggctgctggccaagctttatagccctcaccatttgcctaaggaggtagcagcaatccctaatatatatatatagtgagaactaaaatggatatatttttataatgcagaagaaggaaagtccccctgtgtggtaactgtattgttctagaaatatgctttctagagatatgatgattttgaaactgatttctagaaaaagctgactccatttttgtccctggcgggtaaattaggaatctgcactattttggaggacaagtagcacaaactgtataacggtttatgtccgtagttttatagtcctatttgtagcattcaatagctttattccttagatggttctagggtgggtttacagctttttgtacttttacctccaataaagggaaaatgaagctttttatgtaaattggttgaaaggtctagttttgggaggaaaaaagccgtagtaagaaatggatcatatatattacaactaacttcttcaactatggactttttaagcctaatgaaatcttaagtgtcttatatgtaatcctgtaggttggtacttcccccaaactgattataggtaacagtttaatcatctcacttgctaacatgtttttatttttcactgtaaatatgtttatgttttatttataaaaattctgaaatcaatccatttgggttggtggtgtacagaacacacttaagtgtgttaacttgtgacttctttcaagtctaaatgatttaataaaactttttttaaattaagaaaaaaaaaa

SEQ ID NO: 11 - DNA, 20 bp, Artificial Sequence
  A synthetic oligopeptide
caacatctccggatcttgca

SEQ ID NO: 12 - DNA, 20 bp, Artificial Sequence
  A synthetic oligopeptide
tgggtacaccggcctcatgt

SEQ ID NO: 13 - DNA, 20 bp, Artificial Sequence
  A synthetic oligopeptide
tgatgcacggaagccatcca

SEQ ID NO: 14 - DNA, 20 bp, Artificial Sequence
  A synthetic oligopeptide
taaaggtttcggttgctgac

SEQ ID NO: 15 - DNA, 20 bp, Artificial Sequence
  A synthetic oligopeptide
agagctgtcggacctcgcag

SEQ ID NO: 16 - DNA, 20 bp, Artificial Sequence
  A synthetic oligopeptide
ccatttcgtcactatcccat

SEQ ID NO: 17 - DNA, 20 bp, Artificial Sequence
  A synthetic oligopeptide
gggtatagcacccataacga

SEQ ID NO: 18 - DNA, 20 bp, Artificial Sequence
  A synthetic oligopeptide
gttggccaacctattggtca

SEQ ID NO: 19 - DNA, 20 bp, Artificial Sequence
  A synthetic oligopeptide
tagcacatggctccttcgtc

SEQ ID NO: 20 - DNA, 20 bp, Artificial Sequence
  A synthetic oligopeptide
cacaagcctcactgcgtcgg

SEQ ID NO: 21 - DNA, 20 bp, Artificial Sequence
  A synthetic oligopeptide
tgctactacaacgagaccat

SEQ ID NO: 22 - DNA, 20 bp, Artificial Sequence
  A synthetic oligopeptide
tgagcatgaccacgcggcca

SEQ ID NO: 23 - DNA, 20 bp, Artificial Sequence
  A synthetic oligopeptide
tgacgtacacgtagatccgc

SEQ ID NO: 24 - DNA, 20 bp, Artificial Sequence
  A synthetic oligopeptide
caacttgctggttatcgccg

SEQ ID NO: 25 - DNA, 20 bp, Artificial Sequence
  A synthetic oligopeptide
gatactgtcgatgactggac

SEQ ID NO: 26 - DNA, 20 bp, Artificial Sequence
  A synthetic oligopeptide
gatctcgtactattaggact

SEQ ID NO: 27 - DNA, 20 bp, Artificial Sequence
  A synthetic oligopeptide
tttacaacttcaaccgccac

SEQ ID NO: 28 - DNA, 20 bp, Artificial Sequence
  A synthetic oligopeptide
aaggcttctccaaacgtgtc

SEQ ID NO: 29 - DNA, 20 bp, Artificial Sequence
  A synthetic oligopeptide
cgacctcctgtgccagacga

SEQ ID NO: 30 - DNA, 20 bp, Artificial Sequence
  A synthetic oligopeptide
cggcgggcacggcaaacacc

SEQ ID NO: 31 - DNA, 20 bp, Artificial Sequence
  A synthetic oligopeptide
taggtcggtagtcaggacac

SEQ ID NO: 32 - DNA, 20 bp, Artificial Sequence
  A synthetic oligopeptide
gtgtggttaactgtgatcgg

SEQ ID NO: 33 - DNA, 20 bp, Artificial Sequence
  A synthetic oligopeptide
acaacacggaattggccatt

SEQ ID NO: 34 - DNA, 20 bp, Artificial Sequence
  A synthetic oligopeptide
aaatcgatctacactaatac

SEQ ID NO: 35 - DNA, 20 bp, Artificial Sequence
  A synthetic oligopeptide
tctccatggaccaacacatc

SEQ ID NO: 36 - DNA, 20 bp, Artificial Sequence
  A synthetic oligopeptide
cttccctaatctgtatacgc

SEQ ID NO: 37 - DNA, 20 bp, Artificial Sequence
  A synthetic oligopeptide
atcggcgtcaatctctgctt

SEQ ID NO: 38 - DNA, 20 bp, Artificial Sequence
  A synthetic oligopeptide
ggcaacggccgaagtgaccg

SEQ ID NO: 39 - DNA, 20 bp, Artificial Sequence
  A synthetic oligopeptide
ccgatgacgacgtggaactg

SEQ ID NO: 40 - DNA, 20 bp, Artificial Sequence
  A synthetic oligopeptide
agatggacgaatcgctgcac

SEQ ID NO: 41 - DNA, 20 bp, Artificial Sequence
  A synthetic oligopeptide
ccttcttttataaccggagt

SEQ ID NO: 42 - DNA, 20 bp, Artificial Sequence
  A synthetic oligopeptide
tccatacacgaatgagcaac

SEQ ID NO: 43 - DNA, 20 bp, Artificial Sequence
  A synthetic oligopeptide
cgtagattgccaccatgacc

SEQ ID NO: 44 - DNA, 20 bp, Artificial Sequence
  A synthetic oligopeptide
tagacgggtggaacgcatgg

SEQ ID NO: 45 - DNA, 20 bp, Artificial Sequence
  A synthetic oligopeptide
tgctactacaacgagaccat

SEQ ID NO: 46 - DNA, 20 bp, Artificial Sequence
  A synthetic oligopeptide
tagggcccacgcagccaagt

SEQ ID NO: 47 - DNA, 20 bp, Artificial Sequence
  A synthetic oligopeptide
acggtcaacgttttcgacac

SEQ ID NO: 48 - DNA, 20 bp, Artificial Sequence
  A synthetic oligopeptide
cttgtgatcgtcctgtgcgt

SEQ ID NO: 49 - DNA, 20 bp, Artificial Sequence
  A synthetic oligopeptide
aggcaattccatcccagcgt

SEQ ID NO: 50 - DNA, 20 bp, Artificial Sequence
  A synthetic oligopeptide
gatcgcgtaccatcaggacc

SEQ ID NO: 51 - DNA, 20 bp, Artificial Sequence
  A synthetic oligopeptide
aaggcttctccaaacgtgtc

SEQ ID NO: 52 - DNA, 20 bp, Artificial Sequence
  A synthetic oligopeptide
tttacaactttaatcgccac

SEQ ID NO: 53 - DNA, 20 bp, Artificial Sequence
  A synthetic oligopeptide
catcaacgtggaccgctatg

SEQ ID NO: 54 - DNA, 20 bp, Artificial Sequence
  A synthetic oligopeptide
ggagaccagtcgccaatacc

SEQ ID NO: 55 - DNA, 20 bp, Artificial Sequence
  A synthetic oligopeptide
gatgttcttgtacgtgcagt

SEQ ID NO: 56 - DNA, 20 bp, Artificial Sequence
  A synthetic oligopeptide
gaacgtaacttgttctagta

SEQ ID NO: 57 - DNA, 20 bp, Artificial Sequence
  A synthetic oligopeptide
ggagtcgtcataagggcact

SEQ ID NO: 58 - DNA, 20 bp, Artificial Sequence
  A synthetic oligopeptide
gcaacacggaattggccatt

(19) United States
(12) Patent Application Publication
(10) Pub. No.: US 2022/0273752 A1
(43) Pub. Date: Sep. 1, 2022

(54) MODULATION OF DENDRITIC CELL FUNCTION BY THE PHOSPHOLIPID MESSENGER LPA

(71) Applicant: Cornell University, Ithaca, NY (US)

(72) Inventors: Juan Rodrigo Cubillos Ruiz, New York, NY (US); Chang-Suk Chae, New York, NY (US)

(21) Appl. No.: 17/622,634

(22) PCT Filed: Jul. 2, 2020
(86) PCT No.: PCT/US2020/040665

§ 371 Date: Dec. 23, 2021

Related U.S. Application Data
(60) Provisional application No. 62/870,181, filed on Jul. 3, 2019.
(60) Provisional application No. 62/958,573, filed on Jan. 8, 2020.
(60) Provisional application No. 62/962,349, filed on Jan. 17, 2020.

(51) Int. Cl. A61K 38/00; A61K 31/47; A61K 41/00; A61K 31/42; A61K 31/496; A61P 35/04

(52) U.S. Cl. CPC A61K 38/005; A61K 31/47; A61K 41/00; A61K 31/42; A61K 31/496; A61P 35/04

(57) ABSTRACT

Described herein are compositions and methods that include use of PERK inhibitors, inhibitors of enzymes that can synthesize lysophosphatidic acid (LPA), inhibitors of LPA signaling, such as LPA receptor antagonists, deletion/mutation knockout/knock-down) or PERK or LPA receptors, or combinations thereof. Such compositions and methods can increase production of interferon by dendritic cells in subjects suffering from cancer and improve the survival of those subjects.

### PRIORITY APPLICATIONS

This application claims benefit of priority to the filing date of U.S. Provisional Application Ser. No. 62/870,181 (filed Jul. 3, 2019), 62/958,573 (filed Jan. 8, 2020), and 62/962,349 (filed Jan. 17, 2020), the contents of which are specifically incorporated by reference herein in their entireties.

### GOVERNMENT FUNDING

This invention was made with government support under W81XWH-16-1-0438 awarded by the Department of Defense. The government has certain rights in the invention.

### INCORPORATION BY REFERENCE OF SEQUENCE LISTING PROVIDED AS A TEXT FILE

A Sequence Listing is provided herewith as a text file, “2053719.txt” created on Jul. 1, 2020 and having a size of 53.248 bytes. The contents of the text file are incorporated by reference herein in their entirety.

### BACKGROUND

Cancer is an uncontrolled growth of abnormal cells in various parts of the body. Presently cancer may be treated by surgery, radiotherapy, chemotherapy, immunotherapy, etc., with varying degrees of success. However, surgical therapy cannot completely remove extensively metastasized tumor cells. Radiotherapy and chemotherapy do not have sufficient selectivity to kill cancer cells in the presence of rapidly proliferating normal cells. Immunotherapy is largely limited to the use of cytokines, neutralizing antibodies (checkpoint blockers), therapeutic cancer vaccines or adoptive transfer of cancer-reactive T cells. Cytokines, checkpoint inhibitors and adoptive immunotherapies may cause serious toxicity, and continuous use of vaccines may lead to immune tolerance.

### SUMMARY

Described herein are compositions and methods that inhibit the synthesis and/or functioning of lysophosphatidic acid (LPA) and/or the Endoplasmic Reticulum (ER) stress sensor PERK and/or enzymes involved in in vivo. Surprisingly, such inhibition increases type-I interferon expression in dendritic cells within a mammalian subject. As shown herein, increasing type-I interferon expression or function in dendritic cells by using Applicants' compositions and methods extends overall survival in subjects with aggressive forms of cancer.

For example, composition are described herein that include one or more inhibitors of: (a) lysophosphatidic acid (LPA) production, (b) LPA receptor(s), (c) PERK activation, or (d) a combination of such inhibitors in an amount effective for increasing type-I interferon expression in dendritic cells within a mammalian subject.

Methods are also described herein that include administering to a subject a composition that includes one or more inhibitors of LPA production, one or more inhibitors of one or more LPA receptors, one or more inhibitors of PERK activation, or a combination thereof. The compositions can be administered in an amount effective for increasing type-I interferon expression and/or function. The results of such administration include reducing the progression of cancer, reducing the tumor load, and prolonging the survival of the subject to whom the compositions were administered.

In another example, methods described herein can include: a) obtaining dendritic cells from a subject, b) deleting at least a portion of an endogenous PERK (also known as EIF2AK3) gene, at least a portion of an endogenous autotaxin-encoding (Enpp2) gene, at least a portion of one or more LPAR-encoding genes, or a combination thereof in one or more dendritic cells to generate one or more PERK-defective, Enpp2-defective, or LPAR-defective dendritic cells; and c) administering a population of the PERK-defective, Enpp2-defective, or LPAR-defective dendritic cells to the subject. Such methods can also reduce the progression of cancer, reduce the tumor load, and prolong the survival of the subject to whom the compositions were administered.

Other methods and compositions are also described herein.

### DESCRIPTION OF THE FIGURES

FIG. 1 illustrates relative expression levels of genes encoding lysophosphatidic acid (LPA) receptors in the indicated murine dendritic cell (DC) populations, as determined by RNA-seq. Ovarian cancer DCs were sorted from tumor locations of mice bearing ID8-based metastatic ovarian carcinoma for 24 days.

FIG. 2A-2B illustrate that LPA exposure induces intracellular lipid accumulation in dendritic cells. FIG. 2A shows FACS analysis of bone marrow-derived dendritic cells (BMDCs) exposed to 100 μM LPA for 6 hours. Bodipy 493/503 staining was used to detect intracellular lipids by FACS. ***P<0.0005. FIG. 2B graphically illustrates quantities of Bodipy-stained intracellular lipids in LPA-treated cells compared to untreated cells.

FIG. 3A-3C illustrate that LPA inhibits the antigen-presenting capacity of dendritic cells. Bone marrow-derived dendritic cells (BMDCs) were exposed to 100 μM LPA for 6 hours and then pulsed with full-length ovalbumin (OVA) for 3 hours.

Cells were washed and co-cultured with OVA-specific OT-II T cells labeled with carboxyfluorescein succinimidyl ester (CFSE, which stains intracellular molecules, typically lysine residues). T cell proliferation was assessed by FACS 3 days later.

FIG. 3A shows representative FACS data at various CFSE levels. FIG. 3B graphically illustrates the percentage of OT-II T cells exhibiting cell division. FIG. 3C graphically illustrates the division index of proliferating cells as determined by FlowJo analysis. ****P<0.0001.

FIG. 4A-4L graphically illustrate that induction of a set of highly tumorigenic and immunomodulatory genes is mainly PERK-dependent in LPA-exposed dendritic cells undergoing endoplasmic reticulum (ER) stress. Bone marrow-derived dendritic cells (BMDCs) from the indicated genotypes were left untreated or were incubated with LPA (100 μM), Tunicamycin (TM, 1 μg/ml) or with the combination of both for 6 hours. Gene expression was determined by qPCR. In all cases data, was normalized to endogenous Actb levels in each sample. These findings have further been confirmed using primary splenic dendritic cells and with an independent ER stressor, Thapsigargin (extensive data not shown). ***P<0.0005, ***P<0.0001. The serine/threonine-protein kinase/endoribonuclease inositol-requiring enzyme 1 α (IRE1α) in humans is encoded by the ERNI gene, and expression of the IRE1α protein is activated during endoplasmic reticulum (ER) stress. TM is a pharmacological ER stressor that causes protein glycosylation defects. FIG. 4A graphically illustrates expression of IL-1, in bone marrow-derived DCs from Ern1^{f/f} or Ern1^{f/f} Vav1-Cre (Ern1 knockout) mice with or without co-treatment by ER stressor Tunicamycin (TM) and/or physiological concentrations of LPA. FIG. 4B graphically illustrates expression of IL-6 in bone marrow-derived DCs from Ern1 or Ern1^{f/f} Vav1-Cre mice with or without co-treatment by ER stressor Tunicamycin (TM) and/or physiological concentrations of LPA. FIG. 4C graphically illustrates expression of Ptgs2 (Cox-2) in bone marrow-derived DCs co-treated from Ern1^{f/f} or Ern1^{f/f} Vav1-Cre mice with or without treatment by ER stressor Tunicamycin (TM) and/or physiological concentrations of LPA. FIG. 4D graphically illustrates expression of Vegf-α in bone marrow-derived DCs from Ern1^{f/f} or Ern1^{f/f} Vav1-Cre mice with or without co-treatment by ER stressor Tunicamycin (TM) and/or physiological concentrations of LPA. FIG. 4E graphically illustrates expression of IL-1β in bone marrow-derived DCs from Atf6^{f/f} or Atf6^{f/f} Vav1-Cre mice with or without co-treatment by ER stressor Tunicamycin (TM) and/or physiological concentrations of LPA. FIG. 4F graphically illustrates expression of IL-6 in bone marrow-derived DCs from Atf6^{f/f} or Atf6^{f/f} Vav1-Cre mice with or without co-treatment by ER stressor Tunicamycin (TM) and/or physiological concentrations of LPA. FIG. 4G graphically illustrates expression of Ptgs2 in bone marrow-derived DCs co-treated from Atf6^{f/f} or Atf6^{f/f} Vav1-Cre mice with or without treatment by ER stressor Tunicamycin (TM) and/or physiological concentrations of LPA. FIG. 4H graphically illustrates expression of Vegf-α in bone marrow-derived DCs from Atf6^{f/f} or Atf6^{f/f} Vav1-Cre mice with or without co-treatment by ER stressor Tunicamycin (TM) and/or physiological concentrations of LPA. FIG. 4I graphically illustrates expression of IL-1, in bone marrow-derived DCs from Perk^{f/f} or Perk^{f/f} Tek-Cre mice with or without co-treatment by ER stressor Tunicamycin (TM) and/or physiological concentrations of LPA. FIG. 4J graphically illustrates expression of IL-6 in bone marrow-derived DCs from Perk^{f/f} or Perk^{f/f} Tek-Cre mice with or without co-treatment by ER stressor Tunicamycin (TM) and/or physiological concentrations of LPA. FIG. 4K graphically illustrates expression of Ptgs2 in bone marrow-derived DCs co-treated from Perk^{f/f} or Perk^{f/f} Tek-Cre mice with or without treatment by ER stressor Tunicamycin (TM) and/or physiological concentrations of LPA. FIG. 4L graphically illustrates expression of Vegf-α in bone marrow-derived DCs from Perk^{f/f} or Perk^{f/f} Tek-Cre mice with or without co-treatment by ER stressor Tunicamycin (TM) and/or physiological concentrations of LPA.

FIG. 5A-5P graphically illustrate that the ER stress sensor PERK is necessary for the rapid induction of pro-tumoral and immunomodulatory cytokines by LPA-exposed DCs undergoing ER stress. BMDCs of the indicated genotypes were stimulated with LPA (100 μM), TM (1 μg/ml) or the combination of both for 6 h and supernatants were analyzed using Multiplex Cytokine assays. *P<0.05, **P<0.01, ***P<0.0005, ****P<0.0001. FIG. 5A graphically illustrates expression of IL-10 in bone marrow-derived DCs from Perk^{f/f} (Perk-expressing) or Perk^{f/f} Tek-Cre (Perk knockout) mice with or without co-treatment by ER stressor Tunicamycin (TM) and/or physiological concentrations of LPA. FIG. 5B graphically illustrates expression of IL-6 in bone marrow-derived DCs from Perk^{f/f} or Perk^{f/f} Tek-Cre mice with or without co-treatment by ER stressor Tunicamycin (TM) and/or physiological concentrations of LPA. FIG. 5C graphically illustrates expression of Vegf-α in bone marrow-derived DCs co-treated from Perk^{f/f} or Perk^{f/f} Tek-Cre mice with or without treatment by ER stressor Tunicamycin (TM) and/or physiological concentrations of LPA. FIG. 5D graphically illustrates expression of LIF in bone marrow-derived DCs from Perk^{f/f} or Perk^{f/f} Tek-Cre mice with or without co-treatment by ER stressor Tunicamycin (TM) and/or physiological concentrations of LPA. FIG. 5E graphically illustrates expression of M-CSF in bone marrow-derived DCs from Perk^{f/f} or Perk^{f/f} Tek-Cre mice with or without co-treatment by ER stressor Tunicamycin (TM) and/or physiological concentrations of LPA. FIG. 5F graphically illustrates expression of GRO-α in bone marrow-derived DCs from Perk^{f/f} or Perk^{f/f} Tek-Cre mice with or without co-treatment by ER stressor Tunicamycin (TM) and/or physiological concentrations of LPA. FIG. 5G graphically illustrates expression of MIP1-α in bone marrow-derived DCs co-treated from Perk^{f/f} or Perk^{f/f} Tek-Cre mice with or without treatment by ER stressor Tunicamycin (TM) and/or physiological concentrations of LPA. FIG. 5H graphically illustrates expression of IP10 in bone marrow-derived DCs from Perk^{f/f} or Perk^{f/f} Tek-Cre mice with or without co-treatment by ER stressor Tunicamycin (TM) and/or physiological concentrations of LPA. FIG. 5I graphically illustrates expression of INF-α in bone marrow-derived DCs from Perk^{f/f} or Perk^{f/f} Tek-Cre mice with or without co-treatment by ER stressor Tunicamycin (TM) and/or physiological concentrations of LPA. FIG. 5J graphically illustrates expression of RANTES in bone marrow-derived DCs from Perk^{f/f} or Perk^{f/f} Tek-Cre mice with or without co-treatment by ER stressor Tunicamycin (TM) and/or physiological concentrations of LPA. FIG. 5K graphically illustrates expression of TNF-α in bone marrow-derived DCs co-treated from Perk^{f/f} or Perk^{f/f} Tek-Cre mice with or without treatment by ER stressor Tunicamycin (TM) and/or physiological concentrations of LPA. FIG. 5L graphically illustrates expression of MCP3 in bone marrow-derived DCs from Perk^{f/f} or Perk^{f/f} Tek-Cre mice with or without co-treatment by ER stressor Tunicamycin (TM) and/or physiological concentrations of LPA. FIG. 5M graphically illustrates expression of MCP1 in bone marrow-derived DCs from Perk^{f/f} or Perk^{f/f} Tek-Cre mice with or without co-treatment by ER stressor Tunicamycin (TM) and/or physiological concentrations of LPA. FIG. 5N graphically illustrates expression of MIP2 in bone marrow-derived DCs co-treated from Perk^{f/f} or Perk^{f/f} Tek-Cre mice with or without treatment by ER stressor Tunicamycin (TM) and/or physiological concentrations of LPA. FIG. 5O graphically illustrates expression of IL1-α, in bone marrow-derived DCs from Perk^{f/f} or Perk^{f/f} Tek-Cre mice with or without co-treatment by ER stressor Tunicamycin (TM) and/or physiological concentrations of LPA. FIG. 5P graphically illustrates expression of IL-18 in bone marrow-derived DCs from Perk^{f/f} or Perk^{f/f} Tek-Cre mice with or without co-treatment by ER stressor Tunicamycin (TM) and/or physiological concentrations of LPA.

FIG. 6A-6E illustrate that pharmacological inhibition of PERK prevents induction of pro-tumoral factors in LPA-exposed human DCs undergoing ER stress. Monocyte-derived DCs were generated from peripheral human blood and cells were treated for 6 hours with LPA (100 μM), TM (1 μg/ml) or the combination of both, in the presence or absence of the PERK inhibitor AMG PERK 44. Gene expression was subsequently determined by RT-qPCR relative to endogenous ACTB in each sample. FIG. 6A graphically illustrates expression of the PERK-dependent ER stress response gene DDIT3 in monocyte-derived DCs with or without treatment by AMG PERK 44. FIG. 6B graphically illustrates expression of IL6 in monocyte-derived DCs with or without treatment by AMG PERK 44. FIG. 6C graphically illustrates expression of IL1B in monocyte-derived DCs with or without treatment by AMG PERK 44. FIG. 6D graphically illustrates expression of PTGS2 in monocyte-derived DCs with or without treatment by AMG PERK 44. FIG. 6E graphically illustrates expression of VEGFA in monocyte-derived DCs with or without treatment by AMG PERK 44.

FIG. 7A-7B illustrate that conditional PERK deletion in CD11c⁺ immune cells (Perk^{f/f} Cd11c-Cre) delays metastatic ovarian cancer progression. FIG. 7A illustrates survival of female mice of the indicated genotypes (n=8/group) that were intraperitoneally challenged with parental ID8 ovarian cancer cells and host survival was monitored over time. *P<0.05. ****P<0.0001. n=8/group. FIG. 7B graphically illustrates survival of female mice of the indicated genotypes (n=8/group) that were intraperitoneally challenged with variant ovarian cancer cells that are highly aggressive and overexpress VEGFA and Defb29 (ID8-Defb29/Vegf-A). Host survival was monitored over time. *P<0.05. ****P<0.0001. n=8/group. FIG. 8A-8D graphically illustrate expression of LPA/ER stress-induced tumorigenic and immunomodulatory genes by tumor-associated dendritic cells (tDCs) present in ovarian cancer ascites can be diminished using a small-molecule inhibitor targeting autotaxin. FIG. 8A graphically illustrates expression of IL1β (relative to Actb) with and without treatment with 200 nM or 1000 nM of the Autotaxin inhibitor GLPG1690. FIG. 8B graphically illustrates expression of IL6 (relative to Artb) with and without treatment with 200 nM or 1000 nM of the Autotaxin inhibitor GLPG1690. FIG. 8C graphically illustrates expression of Ptgs2 (relative to Actb) with and without treatment with 200 nM or 1000 nM of the Autotaxin inhibitor GLPG1690. FIG. 8D graphically illustrates expression of Vegf-α (relative to Actb) with and without treatment with 200 nM or 1000 nM of the Autotaxin inhibitor GLPG1690.

FIG. 9A-9B illustrate the anti-ovarian cancer effects of treatment with the autotaxin inhibitor GLPG1690. FIG. 9A is a schematic diagram illustrating the treatment of female mice that were intraperitoneally injected with ID8 ovarian cancer cells overexpressing VEGFA and Def29b. FIG. 9B graphically illustrates host survival as monitored over time of the mice treated as described for FIG. 9A. ***P<0.001. ****P<0.0001.

FIG. 10A-10B illustrate that LPA inhibits type-1 IFN signaling in dendritic cells. FIG. 10A shows heatmap analysis of type-I IFN target genes from LPA-treated BMDCs, showing that LPA reduces expression of essentially all of the listed type-I IFN target genes except Xdh. FIG. 10B shows Ingenuity Pathway Analysis (IPA) of RNA-seq highlighting that LPA causes severe downregulation of multiple gene networks commonly induced by type-I IFNs (i.e., the genes listed above the line), while upregulating various immunosuppressive gene programs controlled by NKX2-3, CREB1, PTGER4 and HIF1 (i.e., the genes listed below the line).

FIG. 11A-11H illustrate that LPA downregulates type-I IFN target genes in dendritic cells. BMDCs were left untreated or incubated with LPA (100 uM) for 2 hours and 6 hours, and gene expression was determined by RT-qPCR. **P<0.001, ***P<0.0005, ****P<0.0001. FIG. 11A illustrates downregulation of Ddx58 mRNA (encoding DExD/H-box helicase 58) relative to Actb expression at 2 hours and 6 hours after LPA treatment of BMDCs. FIG. 11B illustrates downregulation of Ifit1 mRNA (encoding interferon-induced protein with tetratricopeptide repeats 1) relative to Actb expression at 2 hours and 6 hours after LPA treatment of BMDCs. FIG. 11C illustrates downregulation of Ifit2 mRNA (encoding interferon-induced protein with tetratricopeptide repeats 2) relative to Actb expression at 2 hours and 6 hours after LPA treatment of BMDCs. FIG. 11D illustrates downregulation of Isg15 mRNA (interferon-stimulated gene 15) relative to Actb expression at 2 hours and 6 hours after LPA treatment of BMDCs. FIG. 11E illustrates downregulation of Ciita mRNA (encoding a Class II Major Histocompatibility Complex Transactivator) relative to Actb expression at 2 hours and 6 hours after LPA treatment of BMDCs. FIG. 11F illustrates downregulation of Oas1a mRNA (encoding 2′-5′ oligoadenylate synthetase 1A) relative to Actb expression at 2 hours and 6 hours after LPA treatment of BMDCs. FIG. 11G illustrates downregulation of Oas1g mRNA (encoding 2′-5′ oligoadenylate synthetase 1G) relative to Actb expression at 2 hours and 6 hours after LPA treatment of BMDCs. FIG. 11H illustrates downregulation of Oas2 mRNA (encoding 2′-5′ oligoadenylate synthetase 2) relative to Actb expression at 2 hours and 6 hours after LPA treatment of BMDCs.

FIG. 12A-12D illustrate that LPA represses IFN-β production by diverse DC types. Splenic dendritic cells (sDCs), bone marrow dendritic cells (BMDCs) and plasmacytoid dendritic cells (pDCs) were stimulated with Poly (I:C) (a high molecular weight, synthetic analog of double-stranded RNA (dsRNA) that is a potent inducer of interferon), LPS (lipopolysaccharide) or CpG ODN1585 (a synthetic immunostimulatory oligonucleotide) in the presence or absence of LPA (10 uM and 100 uM). IFN-β protein expression levels were determined by ELISA. **P<0.001, ***P<0.0005, ****P<0.0001. FIG. 12A illustrates IFN-β protein expression in splenic DCs (sDCs) after treatment with LPA and/or Poly (I:C). FIG. 12B illustrates IFN-β protein expression in bone marrow dendritic cells (BMDCs) after treatment with LPA and/or Poly (I:C). FIG. 12C illustrates IFN-β protein expression in bone marrow dendritic cells (BMDCs) after treatment with LPS and/or LPA. FIG. 12D illustrates IFN-β protein expression in plasmacytoid DCs (pDCs) after treatment with ODN1585 and/or LPA.

FIG. 13A-13D illustrate that LPA prevents expression of type-I IFN-related genes in BMDCs exposed to ovarian cancer cells pre-treated with the PARP inhibitor Talazoparib. RT-qPCR results are shown of type-I IFN target genes in BMDCs after coculture with talazoparib-treated ovarian cancer cells. *P<0.05, **P<0.001, ****P<0.0001. FIG. 13A illustrates downregulation of Ddx58 mRNA (encoding DExD/H-box helicase 58) by LPA relative to Actb expression in BMDCs co-cultured with ovarian cancer cells pre-treated with the PARP inhibitor Talazoparib. Talazoparib is used in the treatment of advanced breast cancer with germline breast cancer (BRCA) mutations. FIG. 13B illustrates downregulation of Isg15 mRNA (interferon-stimulated gene 15) by LPA relative to Actb expression in BMDCs co-cultured with ovarian cancer cells pre-treated with the PARP inhibitor Talazoparib.

FIG. 13C illustrates downregulation of Oas1a mRNA (encoding 2′-5′ oligoadenylate synthetase 1A) by LPA relative to Actb expression in BMDCs co-cultured with ovarian cancer cells pre-treated with the PARP inhibitor Talazoparib. FIG. 13D illustrates downregulation of Oas2 mRNA (encoding 2′-5′ oligoadenylate synthetase 2) by LPA relative to Actb expression in BMDCs co-cultured with ovarian cancer cells pre-treated with the PARP inhibitor Talazoparib.

FIG. 14A-14B shows representative western blots demonstrating phosphorylation of TBK1 and IRF3, which is crucial for type-I IFN expression, is inhibited in LPA-exposed BMDCs stimulated with either LPS or Poly(I:C). FIG. 14A shows a representative western blot demonstrating that phosphorylation of TBK1 and IRF3 is inhibited in LPA-exposed BMDCs stimulated with LPS. FIG. 14B shows a representative western blot demonstrating phosphorylation of TBK1 and IRF3 is inhibited in LPA-exposed BMDCs stimulated with Poly(I:C).

FIG. 15A-15B illustrate that genetic loss of autotaxin (encoded by Enpp2) in the ovarian cancer cell delays malignant progression and increases in vivo host survival. The mice received variant ovarian cancer cells that are highly aggressive and that overexpress VEGFA and Defb29 (ID8-Defb29/Vegf-A). FIG. 15A graphically illustrates results of a first experiment showing the percent survival of female mice that have ovarian cancer cells that lack the Enpp2 gene (Enpp2 sgRNA). Control mice with ovarian cancer cells that have a functional Enpp2 gene (control sgRNA) exhibit reduced survival. FIG. 15B graphically illustrates results of a second experiment showing the percent survival of female mice with ovarian cancer cells that lack the Enpp2 gene (Enpp2 sgRNA) compared to control mice with ovarian cancer cells that have a functional Enpp2 gene. ***P<0.0001. Control sgRNA, scrambled single-guide RNA. Enpp2 sgRNA, autotaxin-targeting single-guide RNA.

FIG. 16A-16F show that mice implanted with Enpp2-deficient ovarian cancer cells have decreased proportions of malignant spheroids in the peritoneal cavity, while demonstrating enhanced infiltration by activated T cells that produce IFNγ in situ. Peritoneal tumors and ascites samples were collected 4 weeks post-tumor challenge and cells were analyzed by FACS. FIG. 16A illustrates that mice implanted with Enpp2-deficient ovarian cancer cells (bottom panel) have decreased proportions of malignant spheroids in the peritoneal cavity, compared to control mice implanted with ovarian cancer cells having wild type Enpp2 (top panel). FIG. 16B illustrates the proportion of tumor-associated CD3⁺CD4⁺ T cells that produce interferon gamma (IFNγ) in ascites fluids. The top panel shows FACS analysis of ascites from mice with control wild type Enpp2 ovarian cancer cells. The bottom panel shows FACS analysis of ascites from mice with Enpp2-deficient ovarian cancer cells. As illustrated, when the ovarian cancer cells are Enpp2-deficient there is enhanced infiltration by activated T cells that produce IFNγ. FIG. 16C illustrates the proportion of tumor-associated CD3⁺CD8α⁺ T cells that produce IFNγ in ascites fluids. The top panel corresponds to FACS analysis of ascites from mice with control Enpp2-sufficient ovarian cancer cells. The bottom panel corresponds to FACS analysis of ascites from mice with Enpp2-deficient ovarian cancer cells. As illustrated, when the ovarian cancer cells are Enpp2-deficient there is enhanced infiltration by CD3⁺CD8α⁺ T cells that produce IFNγ. FIG. 16D graphically illustrates the percentage of high side scatter (SSC^{high}) tumor cells in mice with control ovarian cancer cells (control sgRNA) and in mice with Enpp2-deficient ovarian cancer cells (Enpp2 sgRNA). FIG. 16E graphically illustrates the percentage of antigen-experienced CD44+CD4+ cells (gated for CD3⁺CD4⁺ cells) that express IFNγ in mice with Enpp2-deficient ovarian cancer (Enpp2 sgRNA). For comparison, the percentage of antigen-experienced CD44+CD4+ cells is also shown for control ovarian cancer that express a functional Enpp2 gene (control sgRNA). FIG. 16F graphically illustrates the percentage of antigen-experienced CD44+CD8+ T cells (gated for CD3⁺CD8α⁺ cells) that express IFNγ in mice with control ovarian cancer (control sgRNA), and in mice with Enpp2-deficient ovarian cancer (Enpp2 sgRNA). The data shown are pooled from multiple independent mice, and corresponding statistics are shown. *P<0.05.

FIG. 17A-17C illustrate host survival and disease progression in mice bearing autotaxin-deficient ovarian cancer (Enpp2 sgRNA) and treated with the TLR3 agonist Poly(1:C). FIG. 17A schematically illustrates the experimental scheme and treatment regimen. FIG. 17B graphically illustrates the survival of the indicated groups when treated as described in FIG. 17A. As shown the autotaxin-deficient ovarian cancer (Enpp2 sgRNA) treated with the TLR3 agonist Poly(I:C) exhibit prolonged survival compared to the other groups. FIG. 17C graphically illustrates ascites accumulation over time of the groups of animals treated as described in FIG. 17A. ****P<0.0001.

FIG. 18A-18C illustrate that blockade of the type-I IFN receptor 1 (IFNAR1) abrogates the therapeutic effects Poly-(I:C) in mice bearing autotaxin-deficient ovarian tumors. FIG. 18A schematically illustrates the experimental scheme and treatment regimen employed. FIG. 18B graphically illustrates the percent survival for the groups described in FIG. 18A. As shown, the autotaxin-deficient ovarian cancer (Enpp2 sgRNA) treated with the TLR3 agonist Poly(I:C) exhibit prolonged survival compared to the other groups. FIG. 18C graphically illustrates ascites accumulation over time of the groups of animals treated as described in FIG. 18A. ***P<0.0005, ****P<0.0001.

FIG. 19A-19C illustrate the effects of the PARP inhibitor Talazoparib in mice bearing autotaxin-deficient ovarian cancer cells. FIG. 19A schematically illustrates the experimental scheme and treatment regimen employed. FIG. 19B graphically illustrates the percent survival for the groups described in FIG. 19A. As shown the autotaxin-deficient ovarian cancer (Enpp2 sgRNA) treated with the PARP inhibitor Talazoparib exhibit prolonged survival compared to the other groups. FIG. 19C graphically illustrates ascites accumulation over time of the groups of animals treated as described in FIG. 19A. **P<0.001, ***P<0.0005, ****P<0.0001.

FIG. 20A-20C illustrate the anti-ovarian cancer effects of co-treatment with the autotaxin inhibitor GLPG1690 and the PARP inhibitor Talazoparib. FIG. 20A schematically illustrates the experimental scheme and treatment regimen employed.

FIG. 20B graphically illustrates the percent survival for the groups described in FIG. 20A. As shown, treatment with the autotaxin inhibitor GLPG1690 and the PARP inhibitor Talazoparib exhibit prolonged survival compared to the other groups. FIG. 20C graphically illustrates ascites accumulation over time of the groups of animals treated as described in FIG. 20A. **P<0.001, ***P<0.001. ****P<0.0001.

FIG. 21 graphically illustrates that female mice without PERK in their CD11c⁺ dendritic cells (Eif2ak3^{f/f} Cd11c-Cre) and that have ID8-based ovarian tumors devoid of autotaxin (Enpp2 sgRNA) exhibit significantly improved survival compared to their PERK-expressing littermate controls (Eif2ak3^{f/f}), or with their corresponding isogenic controls harboring scrambled sgRNA (Control sgRNA). Statistical differences were analyzed using the Log-rank test; *P<0.05, **P<0.01, ***P<0.001.

### DETAILED DESCRIPTION

Described herein are compositions and methods that inhibit autotaxin, an enzyme required for lysophosphatidic acid (LPA) production in vivo. Such compositions and methods can be used to modulate dendritic cell function to increase interferon production, and thereby extend overall survival in cancer hosts. In some cases, autotaxin reduction combined with one or more PERK inhibitors, PARP inhibitors, TLR3 agonists, or a combination thereof can further extend survival of cancer patients.

The Examples provided herein show that inhibitors of LPA synthesis can increase type-I interferon expression in dendritic cells in vivo, within a mammalian subject. Surprisingly such increased interferon production improves the survival of cancer patients. Hence, the compositions and methods described herein are effective chemotherapeutic agents and methods.

Type-I interferons (IFNs) are central coordinators of tumor-immune system interactions. Cancer cells differ antigenically from their normal counterparts and emit danger signals that are detectable by the immune system (e.g., tumor-associated antigens, TAAs). Such signals facilitate establishment of a productive and long-lasting immune response against tumor cells.

Type-I-interferons (IFNs) consist of thirteen partially homologous IFN-α cytokines, a single IFN-β and several not yet well characterized single gene products (IFN-ε, IFN-τ, IFN-κ, IFN-ω, IFN-δ and IFN-ζ) all of which are mostly non-glycosylated proteins of 165-200 amino acids. See, e.g., Pestka et al. Immunol Rev 202:8-32 (2004).

Inhibition of autotaxin (encoded by Enpp2) reduces lysophosphatidic acid (LPA) production. LPA is a bioactive lipid present at high concentrations in malignant ascites and serum of ovarian cancer patients (Fang et al., Ann N Y Acad Sci. 905: 188-208 (2000); Fang et al., Biochimica et Biophysica acta, 1582(1-3): 257-64 (2002)). It is also overproduced in multiple other cancer types such as pancreatic, prostate, breast and colorectal cancer, where it operates as a potent messenger that promotes the proliferation and malignant cells (Hu et al., J Natl Cancer Inst. 95(10):733-40 (2003): Yamada et al. J Biol Chem. 279(8):6595-605 3-5 (2004)); Panupinthu et al. Br J Cancer. 102(6):941-6 (2010)). Importantly, overexpression of LPA-controlled gene signatures strongly correlates with poor prognosis in ovarian cancer patients (Murph et al. PLoS One. 4(5):e5583 (2009)). While LPA has been demonstrated to sustain cancer cell viability and aggressiveness, it remains unknown whether this phospholipid also facilitates malignant progression by inhibiting anti-tumor immunity.

As described herein LPA is a tumor-induced lipid mediator that cripples protective anti-cancer immune responses by inhibiting the optimal function of dendritic cells (DCs).

The synthetic pathways for LPA include conversion of phosphatidylcholine (PC) into lysophosphatidylcholine (LPC) by lecithin-cholesterol acyltransferase (LCAT) and phospholipase A (PLA) I enzymes, or by conversion of PC to phosphatidic acid (PA) by phospholipase D (PLD). LPC is then metabolized to produce lysophosphatidic acid (LPA) by the enzyme autotaxin (ATX). Any of these enzymes can be inhibited to reduce the synthesis of LPA. LPA can be broken down into monoacylglycerol (MAC) by a family of lipid phosphate phosphatases (LPPs). Increased synthesis or activity of these phosphatases can also reduce the quantity or concentration of LPA. Such reduction in LPA is an effective cancer treatment, as illustrated herein.

For example, the expression of autotaxin can be reduced by administration of inhibitors of autotaxin such as GLPG1690, nucleic acid inhibitors of autotaxin, and/or knock-down or knockout of the gene encoding autotaxin. In some cases, cells (e.g., dendritic cells) can be removed from a subject, followed by mutation of the endogenous autotaxin gene in the cells to destroy or reduce autotaxin activity, and then administration of the autotaxin-mutated (knockout or knock-down) cells to the subject.

One example of a human autotaxin protein is shown below as SEQ ID NO:1 (see also NCBI accession no. AAA64785.1, which provides information about conserved domains).

| | |
|---|---|
| 1 | MARRSSFQSC QIISLFTFAV QVSICLGFTA HRIKRAEGWE |
| 41 | EGPPTVLSDS PWTNISGSCK GRCFELQEAG PPDCRCDNLC |
| 81 | KSYTSCCHDF DELCLKTARG WECTKDRCGE VRNEENACHC |
| 121 | SEDCLARGDC CTNYQVVCKG ESHWVDDDCE EIKAAECPAG |
| 161 | FVRPPLIIFS VDGFRASYMK KGSKVMPNIE KLRSCGTHSP |
| 201 | YMRPVYPTKT FPNLYTLATG LYPESHGIVG NSMYDPVFDA |
| 241 | TFHLRGREKF NHRWWGGQPL WITATKQGVK AGTFFWSVVI |
| 281 | PHERRILTIL RWLTLPDHER PSVYAFYSEQ PDFSGKHYGP |
| 321 | FGPEESSYGS PFTPAKRPKR KVAPKRRQER PVAPPKKRRR |
| 361 | KIHRMDHYAA ETRQDKMTNP LREIDKIVGQ LMDGLKQLKL |
| 401 | RRCVNVIFVG DHGMEDVTCD RTEFLSNYLT NVDDITLVPG |
| 441 | TLGRIRSKFS NNAKYDPKAI IANLTCKKPD QHFKPYLKQH |
| 481 | LPKRLHYANN RRIEDIHLLV ERRWHVARKP LDVYKKPSGK |
| 521 | CFFQGDHGFD NKVNSMQTVF VGYGPTFKYK TKVPPFENIE |
| 561 | LYNVMCDLLG LKPAPNNGTH GSLNHLLRTN TFRPTMPEEV |
| 601 | TRPNYPGIMY LQSDFDLGCT CDDKVPEKNK LDELNKRLHT |
| 641 | KGSTEERHLL YGRPAVLYRT RYDILYHTDF ESGYSEIFLM |
| 681 | LLWTSYTVSK QAEVSSVPDH LTSCVRPDVR VSPSFSQNCL |
| 721 | AYKNDKQMSY GFLFPPYLSS SPEAKYDAFL VTNMVPMYPA |
| 761 | FKRVWNYFQR VLVKKYASER VGVNVISGPI FDYDYDGLHD |
| 801 | TEDKIKQYVE GSSIPVPTHY YSIITSCLDF TQPADKCDGP |
| 841 | LSVSSFILPH RPDNEESCNS SEDESKWVEE LMKMHTARVR |
| 881 | DIEHLTSLDF FRKTSRSYPE ILTLKTYLHT YESIE |

A cDNA sequence encoding the human autotaxin protein is available from the NCBI database as accession no. W35594.1, which also provide primer information (see, www.ncbi.nlm.nih.gov/nuccore/537905). This cDNA sequence that encodes the human autotaxin protein (SEQ ID NO: 1) is shown below as SEQ ID NO:2.

| | |
|---|---|
| 1 | CGTGAAGGCA AAGAGAACAC GCTGCAAAAG GCTTCCAAGA |
| 41 | ATCCTCGACA TGGCAAGGAG GAGCTCGTTC CAGTCGTGTC |
| 81 | AGATAATATC CCTGTTCACT TTTGCCGTTG GAGTCAGTAT |
| 121 | CTGCTTAGGA TTCACTGCAC ATCGAATTAA GAGAGCAGAA |
| 161 | GGATGGGAGG AAGGTCCTCC TACAGTGCTA TCAGACTCCC |
| 201 | CCTGGACCAA CATCTCCGGA TCTTGCAAGG GCAGGTGCTT |
| 241 | TGAACTTCAA GAGGCTGGAC CTCCTGATTG TCGCTGTGAC |
| 281 | AACTTGTGTA AGAGCTATAC CAGTTGCTGC CATGACTTTG |
| 321 | ATGAGCTGTG TTTGAAGACA GCCCGTGGCT GGGAGTGTAC |
| 361 | TAAGGACAGA TGTGGAGAAG TCAGAAATGA AGAAAATGCC |
| 401 | TGTCACTGCT CAGAGGACTG CTTGGCCAGG GGAGACTGCT |
| 441 | GTACCAATTA CCAAGTGGTT TGCAAAGGAG AGTCGCATTG |
| 481 | GGTTGATGAT GACTGTGAGG AAATAAAGGC CGCAGAATGC |
| 521 | CCTGCAGGGT TTGTTCGCCC TCCATTAATC ATCTTCTCCG |
| 561 | TGGATGGCTT CCGTGCATCA TACATGAAGA AAGGCAGCAA |
| 601 | AGTCATGCCT AATATTGAAA AACTAAGGTC TTGTGGCACA |
| 641 | CACTCTCCCT ACATGAGGCC GGTGTACCCA ACTAAAACCT |
| 681 | TTCCTAACTT ATACACTTTG GCCACTGGGC TATATCCAGA |
| 721 | ATCACATGGA ATTGTTGGCA ATTCAATGTA TGATCCTGTA |
| 761 | TTTGATGCCA CTTTTCATCT GCGAGGGCGA GAGAAATTTA |
| 801 | ATCATAGATG GTGGGGAGGT CAACCGCTAT GGATTACAGC |
| 841 | CACCAAGCAA GGGGTGAAAG CTGGAACATT CTTTTGGTCT |
| 881 | GTTGTCATCC CTCACGAGCG GAGAATATTA ACCATATTGC |
| 921 | GGTGGCTCAC CCTGCCAGAT CATGAGAGGC CTTCGGTCTA |
| 961 | TGCCTTCTAT TCTGAGCAAC CTGATTTCTC TGGACACAAA |
| 1001 | TATGGCCCTT TCGGCCCTGA GGAGAGTAGT TATGGCTCAC |
| 1041 | CTTTTACTCC GGCTAAGAGA CCTAAGAGGA AAGTTGCCCC |
| 1081 | TAAGAGGAGA CAGGAAAGAC CAGTTGCTCC TCCAAAGAAA |
| 1121 | AGAAGAAGAA AAATACATAG GATGGATCAT TATGCTGCGG |
| 1161 | AAACTCGTCA GGACAAAATG ACAAATCCTC TGAGGGAAAT |
| 1201 | CGACAAAATT GTGGGGCAAT TAATGGATGG ACTGAAACAA |
| 1241 | CTAAAACTGC GTCGGTGTGT CAACGTCATC TTTGTCGGAG |
| 1281 | ACCATGGAAT GGAAGATGTC ACATGTGATA GAACTGAGTT |
| 1321 | CTTGAGTAAT TACCTAACTA ATGTGGATGA TATTACTTTA |
| 1361 | GTGCCTGGAA CTCTAGGAAG AATTCGATCC AAATTTAGCA |
| 1401 | ACAATGCTAA ATATGACCCC AAAGCCATTA TTGCCAATCT |
| 1441 | CACGTGTAAA AAACCAGATC AGCACTTTAA GCCTTACTTG |
| 1481 | AAACAGCACC TTCCCAAACG TTTGCACTAT GCCAACAACA |
| 1521 | GAAGAATTGA GGATATCCAT TTATTGGTGG AACGCAGATG |
| 1561 | GCATGTTGCA AGGAAACCTT TGGATGTTTA TAAGAAACCA |
| 1601 | TCAGGAAAAT GCTTTTTCCA GGGAGACCAC GGATTTGATA |
| 1641 | ACAAGGTCAA CAGCATGCAG ACTGTTTTTG TAGGTTATGG |
| 1681 | CCCAACATTT AAGTACAAGA CTAAAGTGCC TCCATTTGAA |
| 1721 | AACATTGAAC TTTACAATGT TATGTGTGAT CTCCTGGGAT |
| 1761 | TGAAGCCAGC TCCTAATAAT GGGACCCATG GAAGTTTGAA |
| 1801 | TCATCTCCTG CGCACTAATA CCTTCAGGCC AACCATGCCA |
| 1841 | GAGGAAGTTA CCAGACCCAA TTATCCAGGG ATTATGTACC |
| 1881 | TTCAGTCTGA TTTTGACCTG GGCTGCACTT GTGATGATAA |
| 1921 | GGTAGAGCCA AAGAACAAGT TGGATGAACT CAACAAACGG |
| 1961 | CTTCATACAA AAGGGTCTAC AGAAGAGAGA CACCTCCTCT |
| 2001 | ATGGGCGACC TGCAGTGCTT TATCGGACTA GATATGATAT |
| 2041 | CTTATATCAC ACTGACTTTG AAAGTGGTTA TAGTGAAATA |
| 2081 | TTCCTAATGC TACTCTGGAC ATCATATACT GTTTCCAAAC |
| 2121 | AGGCTGAGGT TTCCAGCGTT CCTGACCATC TGACCAGTTG |
| 2161 | CGTCCGGCCT GATGTCCGTG TTTCTCCGAG TTTCAGTCAG |
| 2201 | AACTGTTTGG CCTACAAAAA TGATAAGCAG ATGTCCTACG |
| 2241 | GATTCCTCTT TCCTCCTTAT CTGAGCTCTT CACCAGAGGC |
| 2281 | TAAATATGAT GCATTCCTTG TAACCAATAT GGTTCCAATG |
| 2321 | TATCCTGCTT TCAAACGGGT CTGGAATTAT TTCCAAAGGG |
| 2361 | TATTGGTGAA GAAATATGCT TCGGAAAGAA ATGGAGTTAA |
| 2401 | CGTGATAAGT GGACCAATCT TCGACTATGA CTATGATGGC |
| 2441 | TTACATGACA CAGAAGACAA AATAAAACAG TACGTGGAAG |
| 2481 | GCAGTTCCAT TCCTGTTCCA ACTCACTACT ACAGCATCAT |
| 2521 | CACCAGCTGT CTGGATTTCA CTCAGCCTGC CGACAAGTGT |
| 2561 | GACGGCCCTC TCTCTGTGTC CTCCTTCATC CTGCCTCACC |
| 2601 | GGCCTGACAA CGAGGAGAGC TGCAATAGCT CAGAGGACGA |
| 2641 | ATCAAAATGG GTAGAAGAAC TCATGAAGAT GCACACAGCT |
| 2681 | AGGGTGCGTG ACATTGAACA TCTCACCAGC CTGGACTTCT |
| 2721 | TCCGAAAGAC CAGCCGCAGC TACCCAGAAA TCCTGACACT |
| 2761 | CAAGACATAC CTGCATACAT ATGAGAGCGA GATTTAACTT |
| 2801 | TCTGAGCATC TGCAGTACAG TCTTATCAAC TGGTTGTATA |
| 2841 | TTTTTATATT GTTTTTGTAT TTATTAATTT GAAACCAGGA |
| 2881 | CATTAAAAAT GTTAGTATTT TAATCCTGTA CCAAATCTGA |
| 2921 | CATATTATGC CTGAATGACT CCACTGTTTT TCTCTAATGC |
| 2961 | TTGATTTAGG TAGCCTTGTG TTCTGAGTAG AGCTTGTAAT |
| 3001 | AAATACTGCA GCTTGAGAAA AAGTGGAAGC TTCTAAATGG |
| 3041 | TGCTGCAGAT TTGATATTTG CATTGAGGAA ATATTAATTT |
| 3081 | TCCAATGCAC AGTTGCCACA TTTAGTCCTG TACTGTATGG |
| 3121 | AAACACTGAT TTTGTAAAGT TGCCTTTATT TGCTGTTAAC |
| 3161 | TGTTAACTAT GACAGATATA TTTAAGCCTT ATAAACCAAT |
| 3201 | CTTAAACATA ATAAATCACA CATTCAGTTT T |

A human autotaxin gene is located on chromosome 8 at about NC_000008.11 (119557077 . . . 119673576, complement; see genomic sequence NCBI accession number NG_029498.3).

Expression of LPA receptors (LPARs) in immune cells can be reduced ex vivo for anti-cancer therapeutic purposes. For example, cells (e.g., dendritic cells) can be removed from a subject, followed by mutation/elimination/silencing of endogenous LPAR-encoding genes in the cells to destroy or reduce LPA signaling, and then the LPAR-mutated (knockout or knock-down) cells can be administered to the subject.

An example of a human LPAR1 sequence is shown below as SEQ ID NO:3 (see also NCBI accession no. NP_001392.2, which provides information about conserved domains).

| | |
|---|---|
| 1 | MAAISTSIPV ISQPQFTAMN EPQCFYNESI AFFYNRSGKH |
| 41 | LATEWNTVSK LVMGLGITVC IFIMLANLLV MVAIYVNRRF |
| 81 | HFPIYYLMAN LAAADFFAGL AYFYLMFNTG PNTRRLVTVS |
| 121 | WLLRQDLIDT SLTASVANLL AIAIERHITV FRQMLHTRMS |
| 161 | NRRVVVVIVV IWTMAIVMGA IPSVGWNCIC DIENCSNMAP |
| 201 | LYSDSYLVFW AIFNLVTFVV MVVLYAHIFG YVRQRTMRMS |
| 241 | RHSSGPRRNR DTMMSLLKTV VIVLGAFIIC WTPGLVLLLL |
| 281 | DVCCPQCDVL AYEKFFLLLA EFNSAMNPII YSYRDKEMSA |
| 321 | TFRQILCCQR SENPTGPTEG SDRSASSLNH TILAGVHSND |
| 361 | HSVV |

A cDNA sequence encoding the human LPAR 1 protein is available from the NCBI database as accession no. NM_001401.4, which also provide primer information (see, www.ncbi.nlm.nih.gov/nuccore/1191017826). A human LPAR1 gene is located on chromosome 9 at about NC_000009.12 (110873252 . . . 111038998, complement).

An example of a human LPAR2 sequence is shown below as SEQ ID NO:4 (see also NCBI accession no. NP_004711.22, which provides information about conserved domains: see www.ncbi.nlm.nih.gov/protein/NP_004711.2).

| | |
|---|---|
| 1 | MVIMGQCYYN ETTGFFYNNS GKELSSHWRP KDVVVVALGL |
| 41 | TVSVLVLLTN LLVIAATASN RRFHQPIYYL LGNLAAADLF |
| 81 | AGVAYLFLMF HTGPRTARIS LEGWFLRQGL LDTSLTASVA |
| 121 | TLLATAVERH RSVMAVQLHS RLPRGRVVML IVGVWVAALG |
| 161 | LGLLPAHSWH CLCALDRCSR MAPLLSRSYL AVWATSSLLV |
| 201 | FLLMVAVYTR IFFYVRRRVQ RMAEHVSCHP RYRETTLSLV |
| 241 | KTVVIILGAF VVCWTPGQVV LLLDGLGCES CNVLAVEKYF |
| 281 | LLLAEANSLV NAAVYSCRDA EMRRTFRRLL CCACLRQSTR |
| 321 | ESVHYTSSAQ GGASTRIMIP ENGHPLMDST L |

A cDNA sequence encoding the human LPAR2 protein is available from the NCBI database as accession no. NM_004720.5, which also provides primer information (see, www.ncbi.nlm.nih.gov/nuccore/183396768). An updated LPAR2 cDNA sequence is available as NCBI accession no. NM_004720.7. A human LPAR2 gene is located on chromosome 19 at about NC_000019.10 (19623655 . . . 19628395, complement).

An example of a human LPAR3 sequence is shown below as SEQ ID NO:5 (see also NCBI accession no. NP_036284.1, which provides information about conserved domains; see www.ncbi.nlm.nih.gov/protein/NP_036284.1).

| | |
|---|---|
| 1 | MNECHYDKHM DFFYNRSNTD TVDDWTGTKL VIVLCVGTFF |
| 41 | CLFIFFSNSL VIAAVIKNRK FHFPFYLLAA NLAAASFFAG |
| 81 | IAYVFLMFNT GPVSKTLTVN RWFLRQGLLD SSLTASLTNL |
| 121 | LVIAVERHMS IMRMRVHSNL TKKRVTLLIL LVWAIAIFMG |
| 161 | AVPTLGWNCL CNISACSSLA PIYSRSYLVF WTVSNLMAFL |
| 201 | IMVVVYLRIY VYVKRKTNVL SPHTSGSISR RRTPMKLMKT |
| 241 | VMTVLGAFVV CWTPGLVVLL LDGLNCRQCG VQHVKRWFLL |
| 281 | LALLNSVVNP IIYSYKDEDM YGTMKKMICC FSQENPERRP |
| 321 | SRIPSTVLSR SDTGSQYIED SISQGAVCNK STS |

A cDNA sequence encoding the human LPAR3 protein is available from the NCBI database as accession no. NM_012152.2, which also provide primer information (see, www.ncbi.nlm.nih.gov/nuccore/183396778). An updated cDNA sequence for this LPAR3 protein is available as NCBI accession no. NM_012152.3. A human LPAR3 gene is located on chromosome 1 at about NC_000001.11 (84811601 . . . 84893206, complement).

An example of a human LPAR4 sequence is shown below as SEQ ID NO:6 (see also NCBI accession no. NP_001264929.1, which provides information about conserved domains; see www.ncbi.nlm.nih.gov/protein/NP_001264929.1).

| | |
|---|---|
| 1 | MGDRRFIDFQ FQSSNSSLRP RLGNATANNT CIVDDSFKYN |
| 41 | LNGAVYSVVF ILGLITNSVS LFVFCFRMKM RSETAIFITN |
| 81 | LAVSDLLFVC TLPFKIFYNF NRHWPFGDTL CKISGTAFLT |
| 121 | NIYGSMLFLT CISVDRFLAI VYPFRSRTIR TRRNSAIVCA |
| 161 | GVWILVLSGG ISASLFSTTN VNNATTTCFE GFSKRVWKTY |
| 201 | LSKITIFIEV VGFIIPLILN VSCSSVVLRT LRKPATLSQI |
| 241 | GTNKKKVLKM ITVHMAVFVV CFVPYNSVLF LYALVRSQAI |
| 281 | TNCFLERFAK IMYPITLCLA TLNCCFDPFI YYFTLESFQK |
| 321 | SFYINAHIRM ESLFKTETPL TTKPSLPAIQ EEVSDQTTNN |
| 361 | GGELMLESTF |

A cDNA sequence encoding the human LPAR4 protein is available from the NCBI database as accession no. NM_001278000.1, which also provide primer information (see, www.ncbi.nlm.nih.gov/nuccore/487439766). A human LPAR4 gene is located on the X chromosome at about NC_000023.11 (78747658 . . . 78758714).

An example of a human LPAR5 sequence is shown below as SEQ ID NO:7 (see also NCBI accession no. NP_065133.1, which provides information about conserved domains; see www.ncbi.nlm.nih.gov/protein/9966879).

| | |
|---|---|
| 1 | MLANSSSTNS SVLPCPDYRP THRLHLVVYS LVLAAGLPLN |
| 41 | ALALWVFLRA LRVHSVVSVY MCNLAASDLL FTLSLPVRLS |
| 81 | YYALHHWPFP DLLCQTTGAI FQMNMYGSCI FLMLINCFRY |
| 121 | AAIVHPLRLR HLRRPRVARL LCLGVWALIL VFAVPAARVH |
| 161 | RPSRCRYRDL EVRLCFESFS DELWKGRLLP LVLLAEALGF |
| 201 | LLPLAAVVYS SGRVFWTLAR PDATQSQRRR KTVRLLLANL |
| 241 | VIFLLCFVPY NSTLAVYGLL RSKLVAASVP ARDRVRGVLM |
| 281 | VMVLLAGANC CLDPLVYYFS AEGFRNTLRG LGTPHRARTS |
| 321 | ATNGTRAALA QSERSAVTTD ATRPDAASQG LLRPSDSHSL |
| 361 | SSFTQCPQDS AL |

A cDNA sequence encoding the human LPAR5 protein is available from the NCBI database as accession no. NM_020400.5, which also provide primer information (see, www.ncbi.nlm.nih.gov/nuccore/NM_020400.5). An updated cDNA sequence for this LPAR5 protein is available as NCBI accession no. NM_020400.6. A human LPAR5 gene is located on chromosome 12 at about NC_000012.12 (6618835 . . . 6635959, complement).

An example of a human LPAR6 sequence is shown below as SEQ ID NO:8 (see also NCBI accession no. NP_001155970.1, which provides information about conserved domains; see www.ncbi.nlm.nih.gov/protein/NP_0011.55970.1).

| | |
|---|---|
| 1 | MVSVNSSHCF YNDSFKTYLY GCMFSMVFVL GLISNCVAIY |
| 41 | IFICVLKVRN ETTTYMINLA MSDLLFVFTL PFRIFYFTTR |
| 81 | NWPFGDLLCK ISVMLFYTNM YGSILFLTCI SVDRFLAIVY |
| 121 | PFKSKTLRTK RNAKIVCTGV WLTVIGGSAP AFVFQSTHSQ |
| 161 | GNNASEACFE NFPEATWKTY LSRIVIFIEI VGFFIPLILN |
| 201 | VTCSSMVLKT LTKPVTLSRS KINKTKVLKM IFVHLIIFCF |
| 241 | CFVPYNINLI LYSLVRTQTF VNCSVVAAVR TMYPITLCIA |
| 281 | VSNCCFDPIV YYFTSDTIQN SIKMKNWSVR RSDFRFSEVH |
| 321 | GAENFTCHNL QTLKSKIFDN ESAA |

A cDNA sequence encoding the human LPAR6 protein is available from the NCBI database as accession no. NM_001162498.1, which also provide primer information (see, www.ncbi.nlm.nih.gov/nuccore/241982707). An updated cDNA sequence for this LPAR6 protein is available as NCBI accession no. NM_001162498.3. A human LPAR6 gene is located on chromosome 13 at about NC_000013.11 (48400897 . . . 48444669, complement).

Also as illustrated herein, knockout, knockdown, or inhibition of PERK is an effective cancer treatment, especially when combined with inhibition of LPA. An example of a human PERK amino acid sequence is shown below as SEQ ID NO:9 (see also NCBI accession no. NP_004827.4, which provides information about conserved domains: see www.ncbi.nlm.nih.gov/protein/NP_004827.4).

| | |
|---|---|
| 1 | MERAISPGLL VRALLLLLLL LGLAARTVAA GRARGLPAPT |
| 41 | AEAAFGLGAA AAPTSATRVP AAGAVAAAEV TVEDAEALPA |
| 81 | AAGEQEPRGP EPDDETELRP RGRSLVIIST LDGRIAATDP |
| 121 | ENHGKKQWDL DVGSGSLVSS SLSKPEVFGN KMIIPSLDGA |
| 161 | LFQWDQDRES METVPFTVES LLESSYKFGD DVVLVGGKSL |
| 201 | TTYGLSAYSG KVRYICSATG CRQWDSDEME QEEDILLLQR |
| 241 | TQKTVRAVGP RSGNEKWNFS VGEFELRYIP DMETRAGFIE |
| 281 | STFKPNENTE ESKIISDVEE QEAATMDIVI KVSVADWKVM |
| 321 | AFSKKGGHLE WEYQECTPIA SAWLLKDGKV IPISLFDDTS |
| 361 | YTSNDDVLED EEDTVEAARG ATENSVYLGM YRGQLYLQSS |
| 401 | VRISEKFPSS PKALESVTNE NAIIPLPTIK WKPLIHSPSR |
| 441 | TPVLVGSDEF DKCLSNDKFS HEEYSNGALS ILQYPYDNGY |
| 481 | YLPYYKRERN KRSTQITVRF LDNPHYNKNI RKKDPVLLLH |
| 521 | WWKEIVATIL FCIIATTFIV RRLFHPHPHR QRKESETQCQ |
| 561 | TENKYDSVSG EANDSSWNDI KNSGYISRYL TDEEPIQCLG |
| 601 | RGGEGVVFEA KNKVDDCNYA IKRIRLPNRE LAREKVMREV |
| 641 | KALAKLEHPG IVRYFNAWLE APPEKWQEKM DEIWLKDEST |
| 681 | DWPLSSPSPM DAPSVKIRRM DPFATKEHIE IIAPSPQRSR |
| 721 | SFSVGISCDQ TSSSESQFSP LEFSGMDHED ISESVDAAYN |
| 761 | LQDSCLTDCD VEDGTMDGND EGHSFELCPS EASPYVRSRE |
| 801 | RTSSSIVFED SGCDNASSKE EPKTNRLHIG NHCANKLTAF |
| 841 | KPTSSKSSSE ATLSISPPRP TTLSLDLTKN TTEKLCQSSP |
| 881 | KVYLYIQMQL CRKENLKDWM NGRCTIEERE RSVCLHIFLQ |
| 921 | IAEAVEFLHS KGLMHRDLKP SNIFFTMDDV VKVGDFGLVT |
| 961 | AMDQDEEEQT VLTPMPAYAR HTGQVGTKLY MSPEQIHGNS |
| 1001 | YSHKVDIFSL GLILFELLYP FSTQMERVRT LTDVRNLKFP |
| 1041 | PLFTQKYPCE YVMVQDMLSP SPMERPEAIN IIENAVFEDL |
| 1081 | DFPGKTVLRQ RSRSLSSSGT KHSRQSNNSH SPLPSN |

A cDNA sequence encoding the human PERK protein is available from the NCBI database as accession no. NM_004836.6, which also provide primer information (see, www.ncbi.nlm.nih.gov/nuccore/927028873). A cDNA sequence that encodes the human PERK protein (SEQ ID NO:9) is shown below as SEQ ID NO: 10.

| | |
|---|---|
| 1 | GGAAAGTCCA CCTTCCCCAA CAAGGCCAGC CTGGGAACAT |
| 41 | GGAGTGGCAG CGGCCGCAGC CAATGAGAGA GCAAACGCGC |
| 81 | GGAAAGTTTG CTCAATGGGC GATGTCCGAG ATAGGCTGTC |
| 121 | ACTCAGGTGG CAGCGGCAGA GGCCGGGCTG AGACGTGGCC |
| 161 | AGGGGAACAC GGCTGGCTGT CCAGGCCGTC GGGGCGGCAG |
| 201 | TAGGGTCCCT AGCACGTCCT TGCCTTCTTG GGAGCTCCAA |
| 241 | GCGGCGGGAG AGGCAGGCGT CAGTGGCTGC GCCTCCATGC |
| 281 | CTGCGCGCGG GGCGGGACGC TGATGGAGCG CGCCATCAGC |
| 321 | CCGGGGCTGC TGGTACGGGC GCTGCTGCTG CTGCTGCTGC |
| 361 | TGCTGGGGCT CGCGGCAAGG ACGGTGGCCG CGGGGCGCGC |
| 401 | CCGTGGCCTC CCAGCGCCGA CGGCGGAGGC GGCGTTCGGC |
| 441 | CTCGGGGCGG CCGCTGCTCC CACCTCAGCG ACGCGAGTAC |
| 481 | CGGCGGCGGG CGCCGTGGCT GCGGCCGAGG TGACTGTGGA |
| 521 | GGACGCTGAG GCGCTGCCGG CAGCCGCGGG AGAGCAGGAG |
| 561 | CCTCGGGGTC CGGAACCAGA CGATGAGACA GAGTTGCGAC |
| 601 | CGCGCGGCAG GTCATTAGTA ATTATCAGCA CTTTAGATGG |
| 641 | GAGAATTGCT GCCTTGGATC CTGAAAATCA TGGTAAAAAG |
| 681 | CAGTGGGATT TGGATGTGGG ATCCGGTTCC TTGGTGTCAT |
| 721 | CCAGCCTTAG CAAACCAGAG GTATTTGGGA ATAAGATGAT |
| 761 | CATTCCTTCC CTGGATGGAG CCCTCTTCCA GTGGGACCAA |
| 801 | GACCGTGAAA GCATGGAAAC AGTTCCTTTC ACAGTTGAAT |
| 841 | CACTTCTTGA ATCTTCTTAT AAATTTGGAG ATGATGTTGT |
| 881 | TTTGGTTGGA GGAAAATCTC TGACTACATA TGGACTCAGT |
| 921 | GCATATAGTG GAAAGGTGAG GTATATCTGT TCAGCTCTGG |
| 961 | GTTGTCGCCA ATGGGATAGT GACGAAATGG AACAAGAGGA |
| 1001 | AGACATCCTG CTTCTACAGC GTACCCAAAA AACTGTTAGA |
| 1041 | GCTGTCGGAC CTCGCAGTGG CAATGAGAAG TGGAATTTCA |
| 1081 | GTGTTGGCCA CTTTGAACTT CGGTATATTC CAGACATGGA |
| 1121 | AACGAGAGCC GGATTTATTG AAAGCACCTT TAAGCCCAAT |
| 1161 | GAGAACACAG AAGAGTCTAA AATTATTTCA GATGTGGAAG |
| 1201 | AACAGGAAGC TGCCATAATG GACATAGTGA TAAAGGTTTC |
| 1241 | GGTTGCTGAC TGGAAAGTTA TGGCATTCAG TAAGAAGGGA |
| 1281 | GGACATCTGG AATGGGAGTA CCAGTTTTGT ACTCCAATTG |
| 1321 | CATCTGCCTG GTTACTTAAG GATGGGAAAG TCATTCCCAT |
| 1361 | CAGTCTTTTT GATGATACAA GTTATACATC TAATGATGAT |
| 1401 | GTTTTAGAAG ATGAAGAAGA CATTGTAGAA GCTGCCAGAG |
| 1441 | GAGCCACAGA AAACAGTGTT TACTTGGGAA TGTATAGAGG |
| 1481 | CCAGCTGTAT CTGCAGTCAT CAGTCAGAAT TTCAGAAAAG |
| 1521 | TTTCCTTCAA GTCCCAAGGC TTTGGAATCT GTCACTAATG |
| 1561 | AAAACGCAAT TATTCCTTTA CCAACAATCA AATGGAAACC |
| 1601 | CTTAATTCAT TCTCCTTCCA GAACTCCTGT CTTGGTAGGA |
| 1641 | TCTGATGAAT TTGACAAATG TCTCAGTAAT GATAAGTTTT |
| 1681 | CTCATGAAGA ATATAGTAAT GGTGCACTTT CAATCTTGCA |
| 1721 | GTATCCATAT GATAATGGTT ATTATCTACC ATACTACAAG |
| 1761 | AGGGAGAGGA ACAAACGAAG CACACAGATT ACAGTCAGAT |
| 1801 | TCCTCGACAA CCCACATTAC AACAAGAATA TCCGCAAAAA |
| 1841 | GGATCCTGTT CTTCTTTTAC ACTGGTGGAA AGAAATAGTT |
| 1881 | GCAACGATTT TGTTTTGTAT CATAGCAACA ACGTTTATTG |
| 1921 | TGCGCAGGCT TTTCCATCCT CATCCTCACA GGCAAAGGAA |
| 1961 | GGAGTCTGAA ACTCAGTGTC AAACTGAAAA TAAATATGAT |
| 2001 | TCTGTAAGTG GTGAAGCCAA TGACAGTAGC TGGAATGACA |
| 2041 | TAAAAAACTC TGGATATATA TCACGATATC TAACTGATTT |
| 2081 | TGAGCCAATT CAATGCCTGG GACGTGGTGG CTTTGGAGTT |
| 2121 | GTTTTTGAAG CTAAAAACAA AGTAGATGAC TGCAATTATG |
| 2161 | CTATCAAGAG GATCCGTCTC CCCAATAGGG AATTGGCTCG |
| 2201 | GGAAAAGGTA ATGCGAGAAG TTAAAGCCTT AGCCAAGCTT |
| 2241 | GAACACCCGG GCATTGTTAG ATATTTCAAT GCCTGGCTCG |
| 2281 | AAGCACCACC AGAGAAGTGG CAAGAAAAGA TGGATGAAAT |
| 2321 | TTGGCTGAAA GATGAAAGCA CAGACTGGCC ACTCAGCTCT |
| 2361 | CCTAGCCCAA TGGATGCACC ATCAGTTAAA ATACGCAGAA |
| 2401 | TGGATCCTTT CGCTACAAAA GAACATATTG AAATCATAGC |
| 2441 | TCCTTCACCA CAAAGAAGCA GGTCTTTTTC AGTAGGGATT |
| 2481 | TCCTGTGACC AGACAAGTTC ATCTGAGAGC CAGTTCTCAC |
| 2521 | CACTGGAATT CTCAGGAATG GACCATGAGG ACATCAGTGA |
| 2561 | GTCAGTGGAT GCAGCATACA ACCTCCAGGA CAGTTGCCTT |
| 2601 | ACAGACTGTG ATGTGGAAGA TGGGACTATG GATGGCAATG |
| 2641 | ATGAGGGGCA CTCCTTTGAA CTTTGTCCTT CTGAAGCTTC |
| 2681 | TCCTTATGTA AGGTCAAGGG AGAGAACCTC CTCTTCAATA |
| 2721 | GTATTTGAAG ATTCTGGCTG TGATAATGCT TCCAGTAAAG |
| 2761 | AAGAGCCGAA AACTAATCGA TTGCATATTG GCAACCATTG |
| 2801 | TGCTAATAAA CTAACTGCTT TCAAGCCCAC CAGTAGCAAA |
| 2841 | TCTTCTTCTG AAGCTACATT GTCTATTTCT CCTCCAAGAC |
| 2881 | CAACCACTTT AAGTTTAGAT CTCACTAAAA ACACCACAGA |
| 2921 | AAAACTCCAG CCCAGTTCAC CAAAGGTGTA TCTTTACATT |
| 2961 | CAAATGCAGC TGTGCAGAAA AGAAAACCTC AAAGACTGGA |
| 3001 | TGAATGGACG ATGTACCATA GAGGAGAGAG AGAGGAGCGT |
| 3041 | GTGTCTGCAC ATCTTCCTGC AGATCGCAGA GGCAGTGGAG |
| 3081 | TTTCTTCACA GTAAAGGACT GATGCACAGG GACCTCAAGC |
| 3121 | CATCCAACAT ATTCTTTACA ATGGATGATG TGGTCAAGGT |
| 3161 | TGGAGACTTT GGGTTAGTGA CTGCAATGGA CCAGGATGAG |
| 3201 | GAAGAGCAGA CGGTTCTGAC CCCAATGCCA GCTTATGCCA |
| 3241 | GACACACAGG ACAAGTAGGG ACCAAACTGT ATATGAGCCC |
| 3281 | AGAGCAGATT CATGGAAACA GCTATTCTCA TAAAGTGGAC |
| 3321 | ATCTTTTCTT TAGGCCTGAT TCTATTTGAA TTGCTGTATC |
| 3361 | CATTCAGCAC TCAGATGGAG AGAGTCAGGA CCTTAACTGA |
| 3401 | TGTAAGAAAT CTCAAATTTC CACCATTATT TACTCAGAAA |
| 3441 | TATCCTTGTG AGTACGTGAT GGTTCAAGAC ATGCTCTCTC |
| 3481 | CATCCCCCAT GGAACGACCT GAAGCTATAA ACATCATTGA |
| 3521 | AAATGCTGTA TTTGAGGACT TGGACTTTCC AGGAAAAACA |
| 3561 | GTGCTCAGAC AnAGGTCTCG CTCCTTGAGT TCATCGGGAA |
| 3601 | CAAAACATTC AAGACAGTCC AACAACTCCC ATAGCCCTTT |
| 3641 | GCCAAGCAAT TAGCCTTAAG TTGTGCTAGC AACCCTAATA |
| 3681 | GGTGATGCAG ATAATAGCCT ACTTCTTAGA ATATGCCTGT |
| 3721 | CCAAAATTGC AGACTTGAAA AGTTTGTTCT TCGCTCAATT |
| 3761 | TTTTTGTGGA CTACTTTTTT TATATCAAAT TTAAGCTGGA |
| 3801 | TTTGGGGGCA TAACCTAATT TGAGCCAACT CCTGAGTTTT |
| 3841 | GCTATACTTA AGGAAAGGGC TATCTTTGTT CTTTGTTAGT |
| 3881 | CTCTTGAAAC TGGCTGCTGG CCAAGCTTTA TAGCCCTCAC |
| 3921 | CATTTGCCTA AGGAGGTAGC AGCAATCCCT AATATATATA |
| 3961 | TATAGTGAGA ACTAAAATGG ATATATTTTT ATAATGCAGA |
| 4001 | AGAAGGAAAG TCCCCCTGTG TGGTAACTGT ATTGTTCTAG |
| 4041 | AAATATGCTT TCTAGAGATA TGATGATTTT GAAACTGATT |
| 4081 | TCTAGAAAAA GCTGACTCCA TTTTTGTCCC TGGCGGGTAA |
| 4121 | ATTAGGAATC TGCACTATTT TGGAGGACAA GTAGCACAAA |
| 4161 | CTGTATAACG GTTTATGTCC GTAGTTTTAT AGTCCTATTT |
| 4201 | GTAGCATTCA ATAGCTTTAT TCCTTAGATG GTTCTAGGGT |
| 4241 | GGGTTTACAG CTTTTTGTAC TTTTACCTCC AATAAAGGGA |
| 4281 | AAATGAAGCT TTTTATGTAA ATTGGTTGAA AGGTCTAGTT |
| 4321 | TTGGGAGGAA AAAAGCCGTA GTAAGAAATG GATCATATAT |
| 4361 | ATTACAACTA ACTTCTTCAA CTATGGACTT TTTAAGCCTA |
| 4401 | ATGAAATCTT AAGTGTCTTA TATGTAATCC TGTAGGTTGG |
| 4441 | TACTTCCCCC AAACTGATTA TAGGTAACAG TTTAATCATC |
| 4481 | TCACTTGCTA ACATGTTTTT ATTTTTCACT GTAAATATGT |
| 4521 | TTATGTTTTA TTTATAAAAA TTCTGAAATC AATCCATTTG |
| 4561 | GGTTGGTGGT GTACAGAACA CACTTAAGTG TGTTAACTTG |
| 4601 | TGACTTCTTT CAAGTCTAAA TGATTTAATA AAACTTTTTT |
| 4641 | TAAATTAAGA AAAAAAAAA |

An updated cDNA sequence for this PERK protein is available as NCBI accession no. NM_004836.7. The human PERK gene is located on chromosome 2 at about NC_000002.12 (88556740 . . . 88627464, complement). As illustrated herein, knockout or inhibition of PERK can improve survival of subjects with cancer.

For example, the activation of PERK can be suppressed by administration of inhibitors of PERK such as AMG PERK 44 (Tocris) and the other PERK inhibitors described herein.

Alternatively, expression of PERK can be reduced with nucleic acid inhibitors of PERK, and/or knock-down or knockout of PERK. For example, cells (e.g., dendritic cells) can be removed from a subject, followed by mutation of the endogenous PERK gene in the cells to destroy or reduce PERK activity, and then administration of the PERK-mutated (knockout or knock-down) cells to the subject. Such inhibition or knockout can improve immune responses against cancer.

### Autotaxin/LPA Inhibitors

A variety of autotaxin inhibitors and other inhibitors of LPA function or LPA biosynthesis can be employed in the compositions and methods described herein.

For example, in some cases the inhibitor is one or more of the following GLPG1690, octanoylglycerol pyrophosphate (DGPP 8.0), 2-[[(E)-octadec-9-enoyl]amino]ethyl dihydrogen phosphate, (S)-phosphoric acid mono-[3-(4-benzyloxy-phenyl)-2-octadec-9-enoylamino-propyl] ester (ammonium salt), Ki16425, 2-(2-(2-aminoacetamido)-3-(2,4-dinitrophenylthio)propanamido)pentanedioic acid (NSC161613), AM152 (chemical name (R)-1-(4′-(3-methyl-4-(((1-phenylethoxy)carbonyl)amino)isoxazol-5-yl)-[1,1′-biphenyl]-4-yl)cyclopropane-1-carboxylic acid), VPC32183 (chemical name [(2R)-2-[[(Z)-Octadec-9-enoyl]amino]-3-[4-(pyridin-3-ylmethoxy)phenyl]propyl] dihydrogen phosphate), VPC12249 ((S)-phosphoric acid mono-[3-(4-benzyloxy-phenyl)-2-octadec-9-enoylamino-propyl]ester), H2L 5765834 (chemical name 2-[3-(4-nitrophenoxy)phenyl]-1,3-dioxoisoindole-5-carboxylic acid). NSC12404 (chemical name 2-[(9-Oxo-9H-fluoren-2-yl)carbamoyl]benzoic acid). GRI977143 (chemical name 2-[[3-(1,3-Dioxo-1H-benz[de]isoquinolin-2(3H)-yl)propyl]thio]-benzoic acid), H2L5547924 (chemical name 4,5-dichloro-2-((9-oxo-9H-fluoren-2-yl)carbamoyl)benzoic acid), H2L5828102 (chemical name 2-((9,10-dioxo-9,10-dihydroanthracen-2-yl)carbamoyl) benzoic acid), H2L5186303 (chemical name (Z,Z)-4,4′-[1,3-Phenylenebis(oxy-4,1-phenyleneimino))]bis[4-oxo-2-butenoic acid), compound 5987411 (chemical name 2-({3-[(3-propoxybenzoyl)amino]-benzoyl}amino)benzoic acid), AM966, AM095, PF-8380. SAR 100842, compound 35, SBJ-Cpd1, PAT-505, PAT-048, GWJ-A-23 (chemical name [4-(decanoylamino)benzyl]phosphonic acid)), GK442. BMP22 (chemical name (bis(monoacylglycerol)phosphate)), PharmAkea-Cpd A-E, aptamer RB014, BrP-LPA, an autotaxin inhibitor/LPA inhibitor with the following structure, where X is halogen (e.g., Br) and R is C15-C17 alkyl.

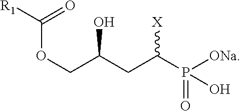

As illustrated herein, some of these inhibitors are more effective than others. In particular, the GLPG1690 is the most effective inhibitor of autotaxin that the inventors have identified for treatment of cancer. This GLPG1690 inhibitor is especially selective for autotaxin and useful for cancer treatment. The GLPG1690 inhibitor has the structure shown below.

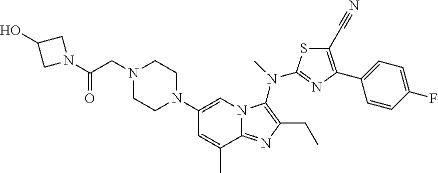 [SMILES: CCc1nc2c(C)cc(N3CCN(CC(=O)N4CC(O)C4)CC3)cn2c1N(C)c1nc(-c2ccc(F)cc2)c(C#N)s1]

GLPG1690 (also called Ziritaxestat) inhibits ATX-induced LPA 18:2 production in mouse, rat, and healthy donor plasma in a concentration-dependent manner, with IC₅₀ values of 418 nM, 542 nM, and 242 nM, respectively.

A structure for Ki16425 is shown below.

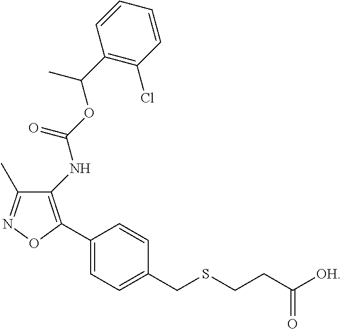 [SMILES: Cc1noc(-c2ccc(CSCCC(=O)O)cc2)c1NC(=O)OC(C)c1ccccc1Cl]

A structure for 2-(2-(2-aminoacetamido)-3-(2,4-dinitrophenylthio)-propanamido)pentanedioic acid (NSC161613) is shown below.

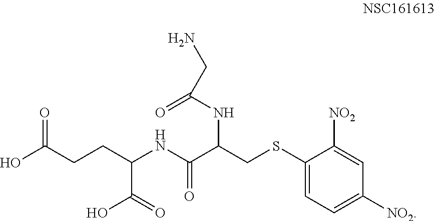 [SMILES: NCC(=O)NC(CSc1ccc([N+](=O)[O-])cc1[N+](=O)[O-])C(=O)NC(CCC(=O)O)C(=O)O]

A structure for AM152 is shown below.

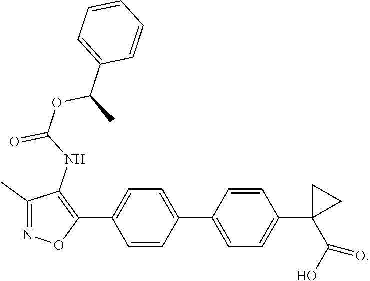 [SMILES: Cc1noc(-c2ccc(-c3ccc(C4(C(=O)O)CC4)cc3)cc2)c1NC(=O)O[C@H](C)c1ccccc1]

A structure for VPC32183 is shown below.

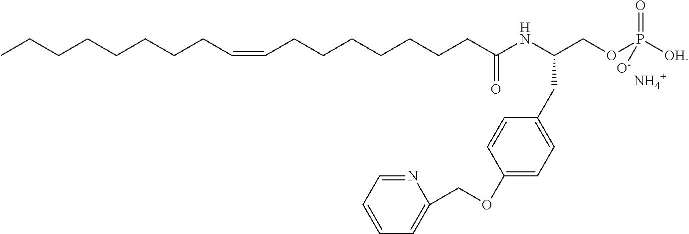 [SMILES: CCCCCCCC/C=C\CCCCCCCC(=O)N[C@H](COP(=O)([O-])O)Cc1ccc(OCc2ccccn2)cc1 ; [NH4+]]

A structure for VPC12249 is shown below.

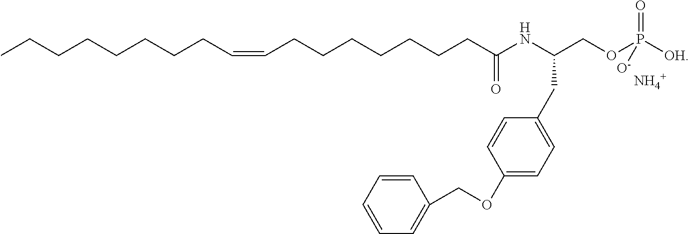 [SMILES: CCCCCCCC/C=C\CCCCCCCC(=O)N[C@H](COP(=O)([O-])O)Cc1ccc(OCc2ccccc2)cc1 ; [NH4+]]

A structure for H2L 5765834 is shown below.

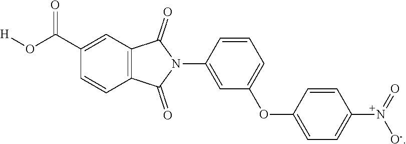

A structure for NSC12404 is shown below.

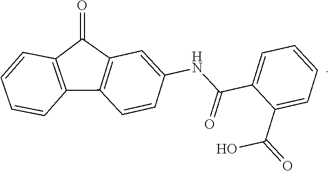 [SMILES: O=C(O)c1ccccc1C(=O)Nc1ccc2c(c1)C(=O)c1ccccc1-2]

A structure for GRI977143 is shown below.

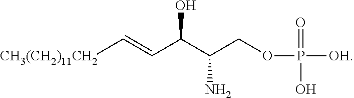

A structure for H2L5547924 (4,5-dichloro-2-((9-oxo-9H-fluoren-2-yl)carbamoyl)benzoic acid) is shown below.

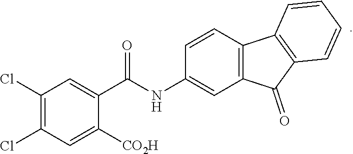 [SMILES: O=C(O)c1cc(Cl)c(Cl)cc1C(=O)Nc1ccc2c(c1)C(=O)c1ccccc1-2]

A structure for H2L5828102 is shown below.

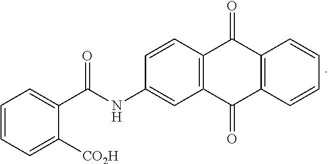 [SMILES: O=C(O)c1ccccc1C(=O)Nc1ccc2c(c1)C(=O)c1ccccc1C2=O]

A structure for H2L5186303 is shown below.

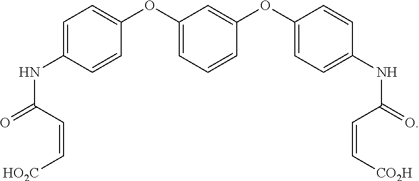 [SMILES: O=C(O)/C=C\C(=O)Nc1ccc(Oc2cccc(Oc3ccc(NC(=O)/C=C\C(=O)O)cc3)c2)cc1]

A structure for compound 5987411 is shown below.

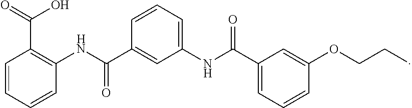

A structure for compound AM966 is shown below.

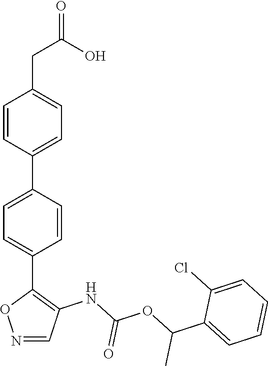 [SMILES: CC(OC(=O)Nc1cnoc1-c1ccc(-c2ccc(CC(=O)O)cc2)cc1)c1ccccc1Cl]

A structure for compound AM095 is shown below.

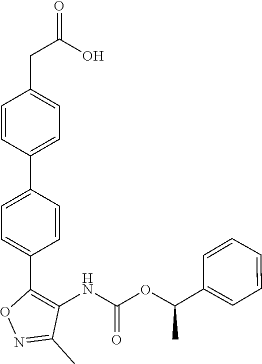 [SMILES: Cc1noc(-c2ccc(-c3ccc(CC(=O)O)cc3)cc2)c1NC(=O)O[C@H](C)c1ccccc1]

A structure for PF-8380 is shown below.

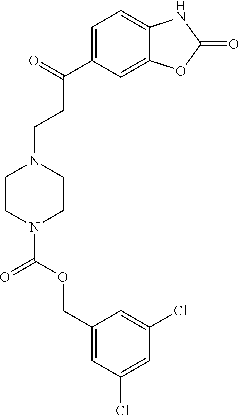 [SMILES: O=C(CCN1CCN(C(=O)OCc2cc(Cl)cc(Cl)c2)CC1)c1ccc2[nH]c(=O)oc2c1]

A structure for SAR 100842 is shown below.

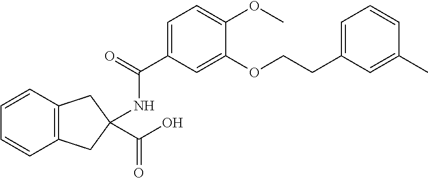 [SMILES: COc1ccc(C(=O)NC2(C(=O)O)Cc3ccccc3C2)cc1OCCc1cccc(C)c1]

A structure for compound 35 is shown below.

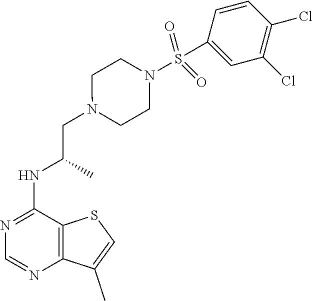 [SMILES: Cc1csc2c(N[C@@H](C)CN3CCN(S(=O)(=O)c4ccc(Cl)c(Cl)c4)CC3)ncnc12]

A structure for SBJ-Cpd1 is shown below.

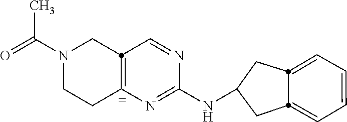

A structure for PAT-505 is shown below.

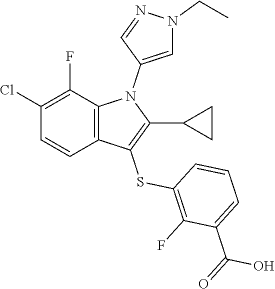 [SMILES: CCn1cc(-n2c(C3CC3)c(Sc3cccc(C(=O)O)c3F)c3ccc(Cl)c(F)c32)cn1]

A structure for PAT-048 is shown below.

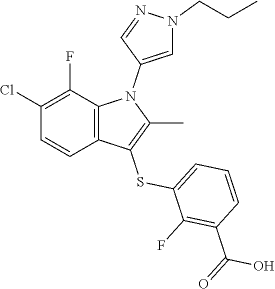 [SMILES: CCCn1cc(-n2c(C)c(Sc3cccc(C(=O)O)c3F)c3ccc(Cl)c(F)c32)cn1]

A structure for GWJ-A-23 is shown below.

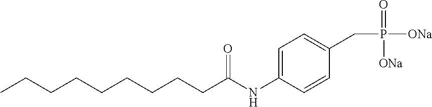

A structure for GK442 is shown below.

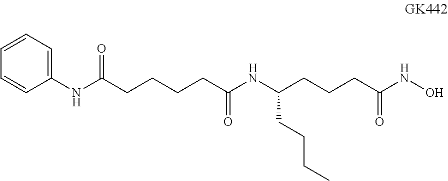 [SMILES: CCCC[C@@H](CCCC(=O)NO)NC(=O)CCCCC(=O)Nc1ccccc1]

A structure for BMP22 is shown below.

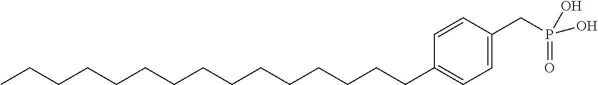 [SMILES: CCCCCCCCCCCCCCCc1ccc(CP(=O)(O)O)cc1]

A structure for the RB014 aptamer is shown below.
PEG-jCCTjGAmCjGmGAAjCCmAmGjAATmAmCjTTjTTGGTjCTjCjCmAmGmjG-idT RB014

A structure for BrP-LPA is shown below.

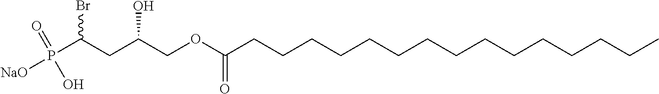

### PERK Inhibitors

A variety of PERK inhibitors can be employed in the compositions and methods described herein.

For example, in some cases the inhibitor is GSK2606414, GSK2656157, AMG52, AMG PERK 44, or a combination thereof.

A structure for GSK2606414 is shown below.

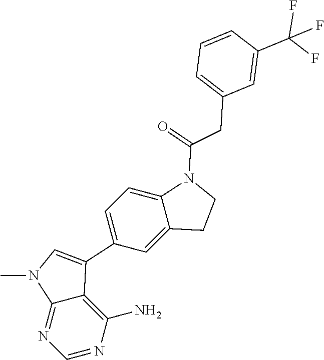 [SMILES: Cn1cc(-c2ccc3c(c2)CCN3C(=O)Cc2cccc(C(F)(F)F)c2)c2c(N)ncnc21]

A structure for GSK2656157 is shown below.

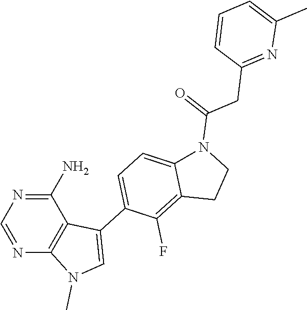 [SMILES: Cc1cccc(CC(=O)N2CCc3c2ccc(-c2cn(C)c4ncnc(N)c24)c3F)n1]

A structure for AMG PERK 52 is shown below.

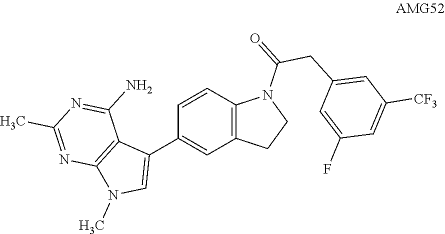 [SMILES: Cc1nc(N)c2c(-c3ccc4c(c3)CCN4C(=O)Cc3cc(F)cc(C(F)(F)F)c3)cn(C)c2n1]

A structure for AMG PERK 44 is shown below.

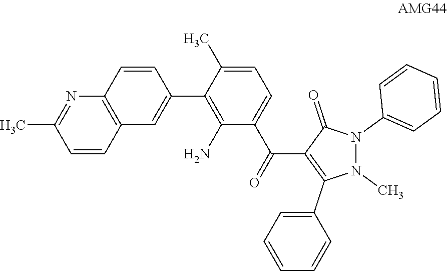 [SMILES: Cc1ccc2cc(-c3c(C)ccc(C(=O)c4c(-c5ccccc5)n(C)n(-c5ccccc5)c4=O)c3N)ccc2n1]

Genomic Modification to Reduce Autotaxin and/or PERK

In some cases, autotaxin, LPA receptor, and/or PERK expression or functioning can be reduced by genomic modification of one or more autotaxin-encoding (Enpp2), LPA receptor, and/or PERK genes.

Non-limiting examples of methods of introducing a modification into the genome of a cell can include use of microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with a guide nucleic acid that allows the nuclease to target the genomic autotaxin (Enpp2), LPA receptor (LPAR), and/or PERK site(s). In some cases, a targeting vector can be used to introduce a deletion or modification of one or more genomic Enpp2, LPA receptor, and/or PERK site(s).

Examples of guide RNA sequences for several genes, including autotaxin (Enpp2), LPA receptor (LPAR), and/or PERK genes are shown below in Tables 1 and 2.

TABLE 1
Guide RNA Sequences for Various Human Genes

| Gene | Position | | |
|---|---|---|---|
| (protein) | (Strand) | Sequence | PAM |
| Enpp2 | 119626678 | CAACATCTCCGGATCTTGCA | AGG |
| (Autotaxin) | (− strand) | (SEQ ID NO: 11) | |
| Enpp2 | 119617199 | TGGGTACACCGGCCTCATGT | AGG |
| (Autotaxin | (+ strand) | (SEQ ID NO: 12) | |
| Enpp2 | 119617511 | TGATGCACGGAAGCCATCCA | CGG |
| (Autotaxin) | (+ strand) | (SEQ ID NO: 13) | |
| eif2ak3 | 88590871 | TAAAGGTTTCGGTTGCTGAC | TGG |
| (PERK) | (− strand) | (SEQ ID NO: 14) | |
| eif2ak3 | 88593283 | AGAGCTGTCGGACCTCGCAG | TGG |
| (PERK) | (− strand) | (SEQ ID NO: 15) | |
| eif2ak3 | 88593350 | CCATTTCGTCACTATCCCAT | TGG |
| (PERK) | (+ strand) | (SEQ ID NO: 16) | |
| Lpar1 | 110941667 | GGGTATAGCACCCATAACGA | TGG |
| | (+ strand) | (SEQ ID NO: 17) | |
| Lpar1 | 110942002 | GTTGGCCAACCTATTGGTCA | TGG |
| | (− strand) | (SEQ ID NO: 18) | |
| Lpar1 | 110941840 | TAGCACATGGCTCCTTCGTC | AGG |
| | (− strand) | (SEQ ID NO: 19) | |
| Lpar2 | 19626929 | CACAAGCCTCACTGCGTCGG | TGG |
| | (− strand) | (SEQ ID NO: 20) | |
| Lpar2 | 19627246 | TGCTACTACAACGAGACCAT | CGG |
| | (− strand) | (SEQ ID NO: 21) | |
| Lpar2 | 19626833 | TGAGCATGACCACGCGGCCA | CGG |
| | (+ strand) | (SEQ ID NO: 22) | |
| Lpar3 | 84865480 | TGACGTACACGTAGATCCGC | AGG |
| | (+ strand) | (SEQ ID NO: 23) | |
| Lpar3 | 84865747 | CAACTTGCTGGTTATCGCCG | TGG |
| | (− strand) | (SEQ ID NO: 24) | |
| Lpar3 | 84866043 | GATACTGTCGATGACTGGAC | AGG |
| | (− strand) | (SEQ ID NO: 25) | |
| Lpar4 | 78755302 | GATCTCGTACTATTAGGACT | AGG |
| | (+ strand) | (SEQ ID NO: 26) | |
| Lpar4 | 78755158 | TTTACAACTTCAACCGCCAC | TGG |
| | (+ strand) | (SEQ ID NO: 27) | |
| Lpar4 | 78755437 | AAGGCTTCTCCAAACGTGTC | TGG |
| | (+ strand) | (SEQ ID NO: 28) | |
| Lpar5 | 6620959 | CGACCTCCTGTGCCAGACGA | CGG |
| | (− strand) | (SEQ ID NO: 29) | |
| Lpar5 | 6620779 | CGGCGGGCACGGCAAACACC | AGG |
| | (+ strand) | (SEQ ID NO: 30) | |
| Lpar5 | 6621187 | TAGGTCGGTAGTCAGGACAC | GGG |
| | (+ strand) | (SEQ ID NO: 31) | |
| Lpar6 | 48411986 | GTGTGGTTAACTGTGATCGG | AGG |
| | (− strand) | (SEQ ID NO: 32) | |
| Lpar6 | 48412172 | ACAACACGGAATTGGCCATT | TGG |
| | (− strand) | (SEQ ID NO: 33) | |
| Lpar6 | 48412078 | AAATCGATCTACACTAATAC | AGG |
| | (+ strand) | (SEQ ID NO: 34) | |

TABLE 2
Guide RNA Sequences for Various Mouse Genes

| Gene | Position | | |
|---|---|---|---|
| (protein) | (Strand) | Sequence | PAM |
| Enpp2 | 54910159 | TCTCCATGGACCAACACATC | TGG |
| (Autotaxin) | (− strand) | (SEQ ID NO: 35) | |
| Enpp2 | 54898895 | CTTCCCTAATCTGTATACGC | TGG |
| (Autotaxin) | (− strand) | (SEQ ID NO: 36) | |
| Enpp2 | 54919654 | ATCGGCGTCAATCTCTGCTT | AGG |
| (Autotaxin) | (− strand) | (SEQ ID NO: 37) | |
| eif2ak3 | 70844907 | GGCAACGGCCGAAGTGACCG | TGG |
| (PERK) | (+ strand) | (SEQ ID NO: 38) | |
| eif2ak3 | 70844987 | CCGATGACGACGTGGAACTG | CGG |
| (PERK) | (+ strand) | (SEQ ID NO: 39) | |
| eif2ak3 | 70858410 | AGATGGACGAATCGCTGCAC | TGG |
| (PERK) | (+ strand) | (SEQ ID NO: 40) | |
| Lpar1 | 58487158 | CCTTCTTTTATAACCGGAGT | GGG |
| | (− strand) | (SEQ ID NO: 41) | |
| Lpar1 | 58486786 | TCCATACACGAATGAGCAAC | CGG |
| | (− strand) | (SEQ ID NO: 42) | |
| Lpar1 | 58487043 | CGTAGATTGCCACCATGACC | AGG |
| | (+ strand) | (SEQ ID NO: 43) | |
| Lpar2 | 69824200 | TAGACGGGTGGAACGCATGG | CGG |
| | (+ strand) | (SEQ ID NO: 44) | |
| Lpar2 | 69823571 | TGCTACTACAACGAGACCAT | CGG |
| | (+ strand) | (SEQ ID NO: 45) | |
| Lpar2 | 69824118 | TAGGGCCCACGCAGCCAAGT | AGG |
| | (− strand) | (SEQ ID NO: 46) | |
| Lpar3 | 146240844 | ACGGTCAACGTTTTCGACAC | CGG |
| | (− strand) | (SEQ ID NO: 47) | |
| Lpar3 | 146240655 | CTTGTGATCGTCCTGTGCGT | GGG |
| | (+ strand) | (SEQ ID NO: 48) | |
| Lpar3 | 146241057 | AGGCAATTCCATCCCAGCGT | GGG |
| | (− strand) | (SEQ ID NO: 49) | |
| Lpar4 | 106930644 | GATCGCGTACCATCAGGACC | AGG |
| | (+ strand) | (SEQ ID NO: 50) | |
| Lpar4 | 106930779 | AAGGCTTCTCCAAACGTGTC | TGG |
| | (+ strand) | (SEQ ID NO: 51) | |
| Lpar4 | 106930500 | TTTACAACTTTAATCGCCAC | TGG |
| | (+ strand) | (SEQ ID NO: 52) | |
| Lpar5 | 125081706 | CATCAACGTGGACCGCTATG | CGG |
| | (+ strand) | (SEQ ID NO: 53) | |
| Lpar5 | 125081457 | GGAGACCAGTCGCCAATACC | AGG |
| | (− strand) | (SEQ ID NO: 54) | |
| Lpar5 | 125081855 | GATGTTCTTGTACGTGCAGT | GGG |
| | (− strand) | (SEQ ID NO: 55) | |
| Lpar6 | 73239196 | GAACGTAACTTGTTCTAGTA | TGG |
| | (+ strand) | (SEQ ID NO: 56) | |
| Lpar6 | 73238622 | GGAGTCGTCATAAGGGCACT | GGG |
| | (− strand) | (SEQ ID NO: 57) | |
| Lpar6 | 73238831 | GCAACACGGAATTGGCCATT | TGG |
| | (+ strand) | (SEQ ID NO: 58) | |

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, the foreign DNA sequence may encode a selectable marker. In some cases, the targeting vector does not comprise a selectable marker, but such a selectable marker can facilitate identification and selection of cells with desirable mutations. Examples of suitable selectable markers include antibiotics resistance genes such as chloramphenicol resistance, gentamycin resistance, kanamycin resistance, spectinomycin resistance (SpecR), neomycin resistance gene (NEO), and/or the hygromycin β-phosphotransferase genes. The 5′ flanking region and the 3′ flanking region can be homologous to regions within the gene, or to regions flanking the gene to be deleted, modified, or replaced with the unrelated DNA sequence.

The targeting vector is contacted with the native gene of interest in vivo (e.g., within the cell) under conditions that favor homologous recombination. For example, the cell can be contacted with the targeting vector under conditions that result in transformation of the cyanobacterial cell(s) with the targeting vector.

A typical targeting vector contains nucleic acid fragments of not less than about 0.1 kb nor more than about 10.0 kb from both the 5′ and the 3′ ends of the genomic locus which encodes the gene to be modified (e.g. the genomic autotaxin (Enpp2), LPA receptor, and/or PERK site(s)). These two fragments are separated by an intervening fragment of nucleic acid which encodes the modification to be introduced. When the resulting construct recombines homologously with the chromosome at this locus, it results in the introduction of the modification, e.g. a deletion of a portion of the genomic autotaxin (Enpp2), LPA receptor, and/or PERK site(s), replacement of the genomic Enpp2, LPA receptor, and/or PERK promoter or coding region site(s), or the insertion of non-conserved codon or a stop codon.

In some cases, a Cas9/CRISPR system can be used to create a modification in genomic autotaxin (Enpp2), LPA receptor, and/or PERK that reduces the expression or functioning of the autotaxin, LPA receptor, and/or PERK polypeptides. Clustered regularly interspaced short palindromic repeats (CRISPR)/CRISPR-associated (Cas) systems are useful for, e.g. RNA-programmable genome editing (see e.g., Marraffini & Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Cuff Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is described, for example, by Mali et al. Science 2013 339:823-6; which is incorporated by reference herein in its entirety. Kits for the design and use of CRISPR-mediated genome editing are commercially available, e.g. the PRECISION X CAS9 SMART NUCLEASE™ System (Cat No. CAS900A-1) from System Biosciences, Mountain View, Calif.

In other cases, a cre-lox recombination system of bacteriophage P1, described by Abremski et al. 1983. Cell 32:1301 (1983), Sternberg et al., Cold Spring Harbor Symposia on Quantitative Biology, Vol. XLV 297 (1981) and others, can be used to promote recombination and alteration of the genomic autotaxin, LPA receptor, and/or PERK site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic mutations so incorporated can alter one or more amino acids in the encoded autotaxin. LPA receptor, and/or PERK gene products. For example, genomic sites modified so that in the encoded autotaxin, LPA receptor, and/or PERK protein is more prone to degradation, or is less stable, so that the half-life of such protein(s) is reduced. In another example, genomic sites can be modified so that at least one amino acid of an autotaxin, LPA receptor, and/or PERK polypeptide is deleted or mutated to reduce the enzymatic activity at least one type of autotaxin, LPA receptor, and/or PERK. In some cases, a conserved amino acid or a conserved domain of the autotaxin, LPA receptor, and/or PERK polypeptide is modified. For example, a conserved amino acid or several amino acids in a conserved domain of the autotaxin, LPA receptor, and/or PERK polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following Table 3.

TABLE 3

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

Such genomic modifications can reduce the expression or functioning of autotaxin, LPA receptor, and/or PERK gene products by at least 10%, or at least 15%, or at least 20%, or at least 25%, or at least 30%, or at least 35%, or at least 40%, or at least 45%, or at least 50%, or at least 60%, or at least 70%, or at least 75%, or at least 80%, or at least 85%, or at least 90%, or at least 95%, or at least 97%, or at least 99%, compared to the unmodified autotaxin, LPA receptor, and/or PERK gene product expression or functioning.

### Methods

The inhibitors of PERK, LPA synthesis, autotaxin or combinations thereof can be administered to a subject. Similarly, immune-related cells such as dendritic cells can be mutated to reduce the activities of autotaxin, LPA sensors (receptors), and/or PERK, and those cells can then be administered to a subject (e.g., the subject from whom the cells were originally obtained).

Hence, methods are described herein can include administering inhibitors of PERK, LPA synthesis, LPA receptor function, autotaxin, or combinations thereof. Such inhibitors of PERK, LPA synthesis, LPA receptors, autotaxin, or combinations thereof can be administered in a composition. The compositions can include a carrier such as a liquid, solvent, or dispersant. Additional description of compositions is provided below.

One method can include: a) obtaining dendritic cells from a subject, b) deleting at least a portion of an endogenous PERK gene (EIF2AK3) in one or more dendritic cells to generate PERK-defective dendritic cells; and c) administering a population of the PERK-defective dendritic cells to the subject. Such a method can also include administering a composition that includes can inhibitors of PERK, LPA synthesis, LPAR antagonists, autotaxin, or combinations thereof to the subject.

Another method can include: a) obtaining dendritic cells from a subject, b) deleting or silencing at least a portion of an endogenous gene that encodes autotaxin or any LPA receptor in one or more dendritic cells to generate dendritic cells unable to produce or sense LPA; and c) administering a population of the autotaxin-deficient or the LPA receptor-defective dendritic cells to the subject. Such a method can also include administering a composition that includes inhibitors of PERK, LPA synthesis, LPAR antagonists, autotaxin inhibitors, or combinations thereof to the subject.

Such methods and the compositions described herein can reduce lysophosphatidic acid (LPA) production or signaling by at least 10%, or at least 20%, or at least 30%, or at least 40%, or at least 50%, or at least 60%, or at least 70%, or at least 80%, or at least 90%, or at least 95% in dendritic cells compared to control untreated dendritic cells.

Such methods and the compositions described herein can reduce expression of at least one of autotaxin, PERK, LPA receptor, IL6, IL1B. PTGS2, or VEGFA by at least 10%, or at least 20%, or at least 30%, or at least 40%, or at least 50%, or least 60%, or at least 70%, or at least 80%, or at least 90%, or at least 95% in dendritic cells compared to control untreated dendritic cells.

Such methods and the compositions described herein can inhibit enzymatic activity of autotaxin by at least 10%, or at least 20%, or at least 30%, or at least 40%, or at least 50%, or at least 60%, or at least 70%, or at least 80%, or at least 90%, or at least 95% in cells or in circulation compared to control untreated hosts.

The methods and compositions described herein can be used to treat a variety of cancers and tumors, for example, breast cancer, colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, pancreatic cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer, and cancer at an unknown primary site. In some cases, the cancer is breast cancer (e.g., triple-negative breast cancer), ovarian cancer, pancreatic cancer, prostate cancer, or a combination thereof.

Another method can include inducing tolerogenic dendritic cells. Such a method can include obtaining dendritic cells from a subject, contacting the dendritic cells with LPA, and then administering the LPA-treated cells to the subject. Bioinformatics analyses have shown that treatment of dendritic cells with LPA dramatically silences the expression of gene signatures involved in type 1 interferon signaling, as well as DDX58 (RIG-1). Such methods can produce tolerogenic dendritic cells. In some cases. LPAR agonists can be administered to a subject to control (e.g., reduce) pro-inflammatory mediators of a variety of diseases such as systemic lupus erythematosus (lupus) (including pediatric lupus), rheumatoid arthritis (RA), psoriasis, psoriatic arthritis, multiple sclerosis, inflammatory bowel disease, Addison's disease, Graves' disease, and the like.

### Compositions

The invention also relates to compositions containing an inhibitor of PERK, inhibitor of autotaxin, and/or an inhibitor of LPA activity or LPA generation or LPA sensing. Such an inhibitor can be a small molecule, an antibody, or a nucleic acid. For example, the nucleic acid inhibitors can inhibit PERK expression, autotaxin expression, LPA synthesis, or LPA receptor expression (e.g., within an expression cassette or expression vector). The compositions of the invention can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the therapeutic agents of the invention (e.g., small molecules, antibodies, inhibitors of PERK, autotaxin, and/or LPA, and/or inhibitory nucleic acids of PERK, autotaxin, and/or enzymes that generate LPA or that encode LPA receptors), are administered in a “therapeutically effective amount.” Such a therapeutically effective amount is an amount sufficient to obtain the desired physiological effect, such a reduction of at least one symptom of cancer. For example, the inhibitors can reduce LPA and PERK activity or synthesis and/or can increase immune responses against cancer cells by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%. Symptoms of cancer can also include tumor cachexia, tumor-induced pain conditions, tumor-induced fatigue, tumor growth, and metastatic spread.

To achieve the desired effect(s), the inhibitors, and combinations thereof, may be administered as single or divided dosages. For example, the inhibitors, can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the small molecules, antibodies or nucleic acid chosen for administration, the disease, the weight, the physical condition, the health, and the age of the mammal. Such factors can be readily determined by the clinician employing animal models or other test systems that are available in the art.

Administration of the therapeutic agents may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents and compositions of the invention may be essentially continuous over a preselected period of time or may be in a series of spaced doses. Both local and systemic administration is contemplated.

To prepare the composition, small molecules, antibodies, nucleic acids, and other agents are synthesized or otherwise obtained, purified as necessary or desired. These molecules, antibodies, nucleic acids, and other agents can be suspended in a pharmaceutically acceptable carrier and/or lyophilized or otherwise stabilized. The small molecules, antibodies, nucleic acid inhibitors or expression, and combinations thereof can be adjusted to an appropriate concentration, and optionally combined with other agents. The absolute weight of a given small molecule, antibody, nucleic acid, and/or another agent included in a unit dose can vary widely. For example, about 0.01 to about 2 g, or about 0.1 to about 500 mg, of at least one molecule, antibody, nucleic acid, and/or other agent, or a plurality of molecules, antibodies, nucleic acids, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the therapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amounts of molecules, antibodies, nucleic acids and/or other agents for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the small molecule(s), antibodies, nucleic acid(s) and/or agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The small molecule(s), antibodies, nucleic acid(s) and/or agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system. For example, the small molecule(s), antibodies, nucleic acid(s) and/or agent(s) can be linked to a convenient carrier such as a nanoparticle, albumin, polyalkylene glycol, or be supplied in prodrug form. The small molecule(s), antibodies, nucleic acid(s) and/or agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the small molecule(s), antibodies, nucleic acid(s) and/or agent(s) can sometimes be administered in an oral dosage form, that oral dosage form can be formulated so as to protect the molecules, peptides, nucleic acids from degradation or breakdown before the small molecule(s), antibodies, nucleic acid(s) and/or agent(s), and combinations thereof provide therapeutic utility. For example, in some cases the small molecule(s), antibodies, nucleic acid(s) and/or agent(s) can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The inhibitors can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

An inhibitor can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as chemotherapeutic agents, anti-viral agents, antibacterial agents, antimicrobial agents and/or preservatives.

Examples of additional therapeutic and/or chemotherapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatgonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The inhibitors can also be used in conjunction with radiation therapy.

The following non-limiting Examples illustrate some aspects of the development of the invention.

### Example 1: Dendritic Cells Express Lysophosphatidic Acid (LPA) Receptors

The Example illustrates LPA receptor expression levels in various dendritic cell populations (BMDCs) and dendritic cells isolated from mice bearing ovarian tumors.

### Methods

Ovarian cancer DCs were sorted from tumor locations of mice bearing ID8-based metastatic ovarian carcinoma for 24 days. The ID8 syngeneic mouse cell line model was derived from C57BL/6 mouse ovarian surface epithelial cells that were transformed by serial passage in vitro (Robey et al. Carcinogenesis 21: 585-591 (2000)). Luciferase was expressed in the ID8 cells (ID8-Luc-mCherry-Puro) to enable monitoring of orthotopic (intraperitoneal) tumor growth by bioluminescence imaging (BLI).

Relative expression levels of genes encoding LPA receptors in the indicated dendritic cell (DC) populations was determined by RNA-seq.

### Results

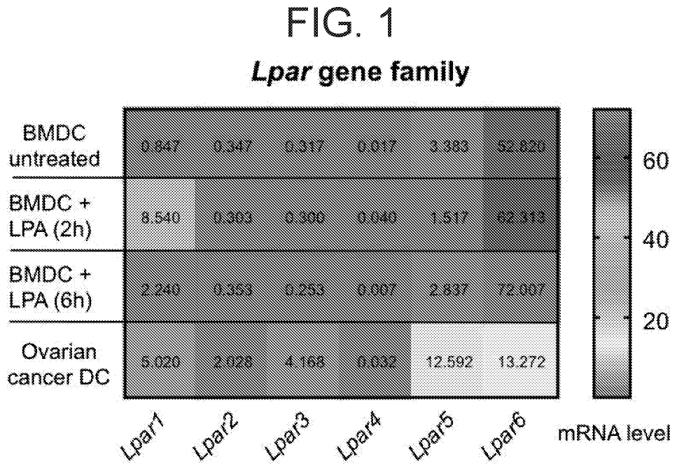

FIG. 1 illustrates that murine bone marrow derived DCs (BMDCs) as well as DCs infiltrating ovarian tumors expressed significant levels of genes encoding various LPA receptors, particularly Lpar6 (see also Table 3).

TABLE 3
LPA Receptor Expression Levels

| | Lpar1 | Lpar2 | Lpar3 | Lpar4 | Lpar5 | Lpar6 |
|---|---|---|---|---|---|---|
| BMDC | 0.847 | 0.347 | 0.317 | 0.017 | 3.383 | 52.820 |
| untreated | | | | | | |
| BMDC + | 8.540 | 0.303 | 0.300 | 0.040 | 1.517 | 62.313 |
| LPA (2 hr.) | | | | | | |
| BMDC + | 2.240 | 0.353 | 0.253 | 0.007 | 2.837 | 72.007 |
| LPA (6 hr.) | | | | | | |
| Ovarian | 5.020 | 2.028 | 4.168 | 0.032 | 12.592 | 13.272 |
| Cancer DCs | | | | | | |

These results indicate that the LPA phospholipid is a messenger that could signal in DCs to influence their function.

### Example 2: Several Gene Networks are Regulated by LPA

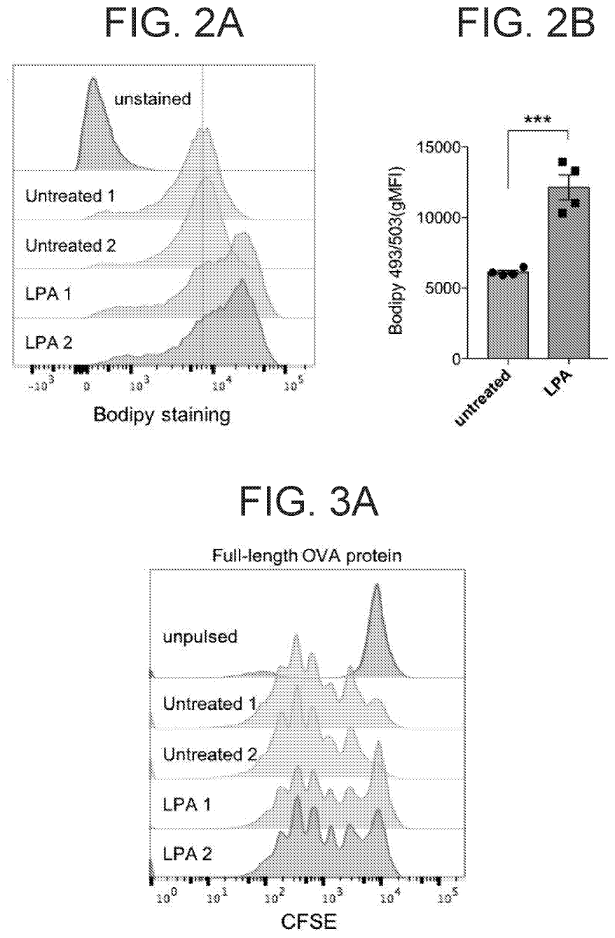

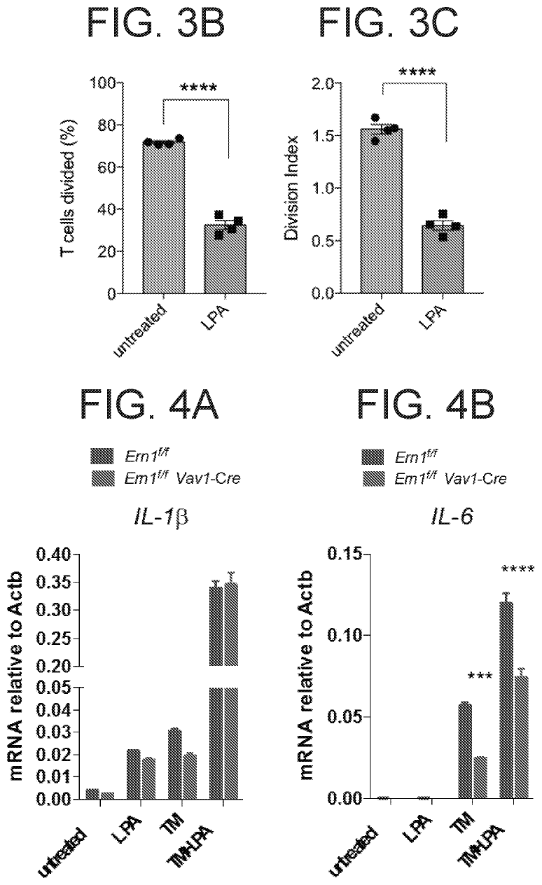

Genome-wide transcriptional profiling using RNA-seq revealed several gene networks regulated by LPA in dendritic cells. LPA concentrations similar to those found in the ascites of metastatic ovarian cancer patients (100 μM) rapidly re-programmed the global transcriptional profile of dendritic cells with nearly 4,000 genes demonstrating severe deregulation. Of particular interest, LPA exposure drastically inhibited genes implicated in the function, quantity and recruitment of antigen-presenting cells, as well as and type-1 Interferon signaling, while upregulating transcriptional processes involved in carbohydrate and lipid metabolism, and expression of immunosuppressive and protumoral genes encoding Arginase, IL-6, IL-1b, Vegf-α and Cox-2 (as assessed by bioinformatic analyses). Accordingly, LPA-driven transcriptional re-programming skewed DCs towards an immunoregulatory phenotype characterized by aberrant intracellular lipid accumulation (FIG. 2) and diminished antigen processing and presenting capacity, which ultimately resulted in defective T cell activation and proliferation in response to specific antigens (FIG. 3).

### Example 3: PERK and LPA are Both Tumorigenic

The inventors have demonstrated that DCs infiltrating ovarian tumors experience detrimental endoplasmic reticulum (ER) stress, a process that disrupts their metabolic homeostasis and that consequently inhibits their normal capacity to activate and stimulate tumor-reactive T cells in situ (Cubillos-Ruiz et al. Cell. 161(7):1527-38 (2015); Cubillos-Ruiz et al. Clin Cancer Res 22(9):2121-6 (2016); Cubillos-Ruiz et al. Cell 168(4):692-706 (2017)).

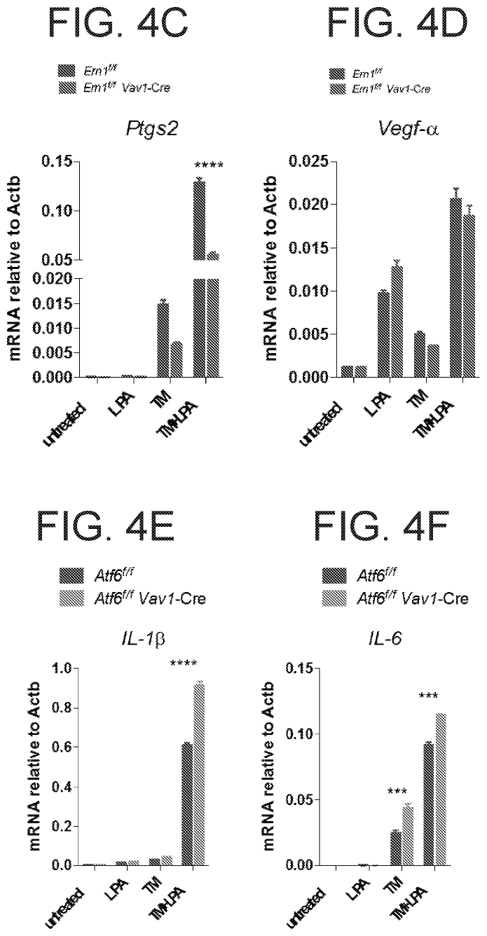

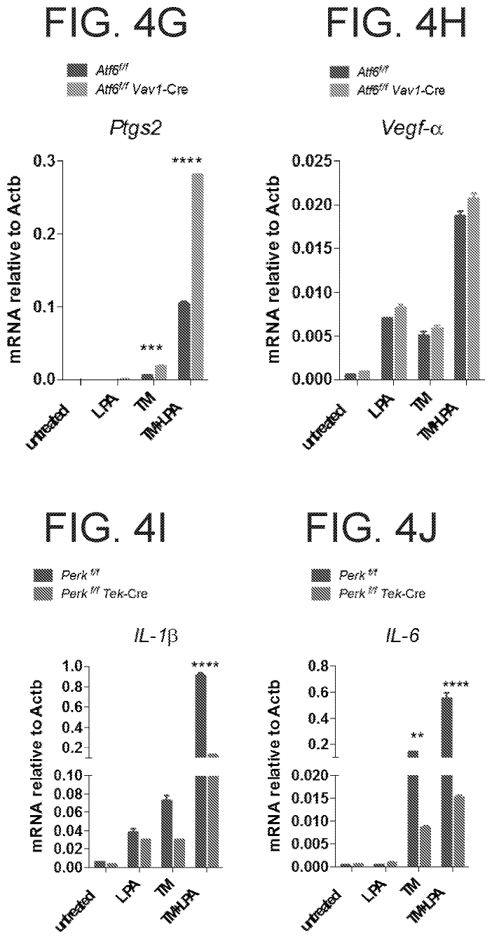

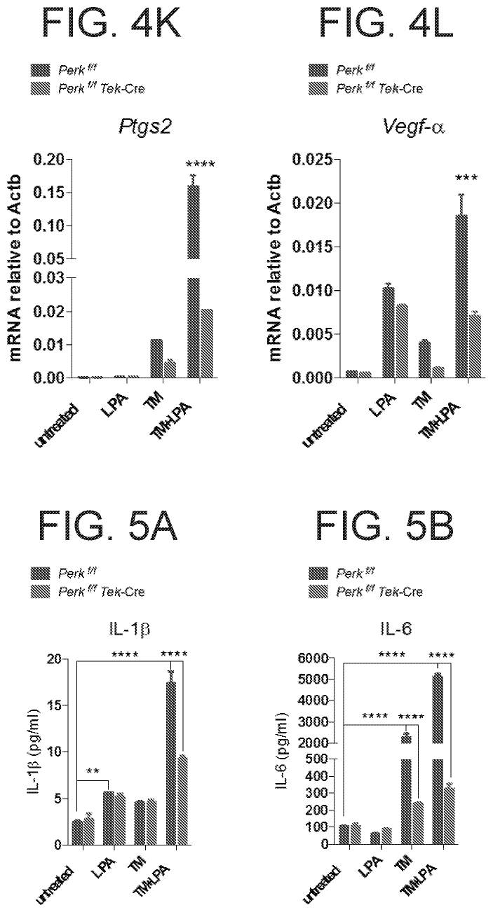

The inventors hypothesized that LPA signaling and ER stress could cooperate to endow DCs with robust tumorigenic and immunosuppressive capacity. In support of this conclusion, ER-stressed DCs simultaneously exposed to LPA demonstrated potent upregulation of genes encoding the immunomodulatory and tumorigenic mediators that were identified by RNA-seq, including IL-6, IL-1b, Arginase, Cox-2 and Vegf-A (FIG. 4). Taken together, these findings indicate that concomitant ER stress and LPA stimulation represents a new mechanism sculpting regulatory dendritic cells in cancer.

The following experiments were performed to determine the precise ER stress sensor (IRE1, PERK or ATF6) that cooperates with LPA signaling to rapidly induce immunoregulatory and protumoral attributes in DCs. Bone marrow derived DCs were generated or splenic dendritic cells were isolated from conditional knockout mice that had selective and independent deletions of each ER stress sensor in their immune cells.

Atf6^{f/f}, Vav1^{cre}: and CD11c^{cre} mice were obtained from The Jackson Laboratory. Xbp1^{f/f} and Ern1^{f/f} mice have been previously described by the inventors (Lee et al. Science 320, 1492 (Jun. 13, 2008); Iwawaki et al. Proc Natl Acad Sci USA 106, 16657 (Sep. 29, 2009)). Conditional knockout mice lacking XBP1, IRE1α or ATF6 in leukocytes were generated by crossing Xbp1^{f/f}, Ern1^{f/f} or Atf6^{f/f} animals, respectively, with the Vav1cre strain that allows selective gene deletion in hematopoietic cells (de Boer et al. Eur J Immunol 33, 314 (February 2003)). Crossing Eif2ak3^{f/f} mice with CD11c^{cre} animals generated mice devoid of PERK in dendritic cells (DC). All mouse strains had a full C57BL/6 background.

Such extensive genetic analysis revealed that PERK is the dominant ER stress sensor that co-operates with LPA signaling to provoke overexpression of factors such as IL-1b. IL-6, Cox-2 and Vegf-A in DCs undergoing ER stress (FIG. 4). This analysis also showed that genetic deletion of the IRE1α arm reduced the expression of the IL-1b, IL-6, Cox-2 and Vegf-A factors, although to a lesser extent than observed for PERK-deficient dendritic cells undergoing ER stress and exposed to LPA (FIG. 4).

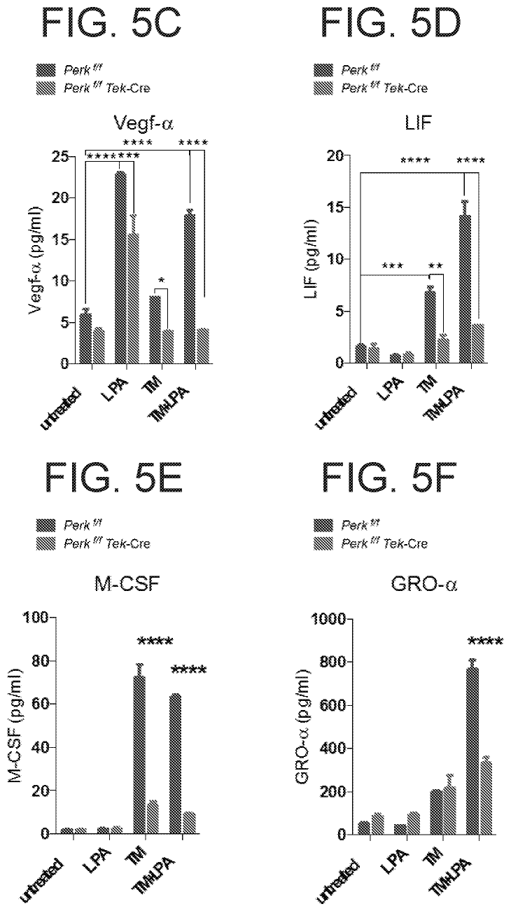

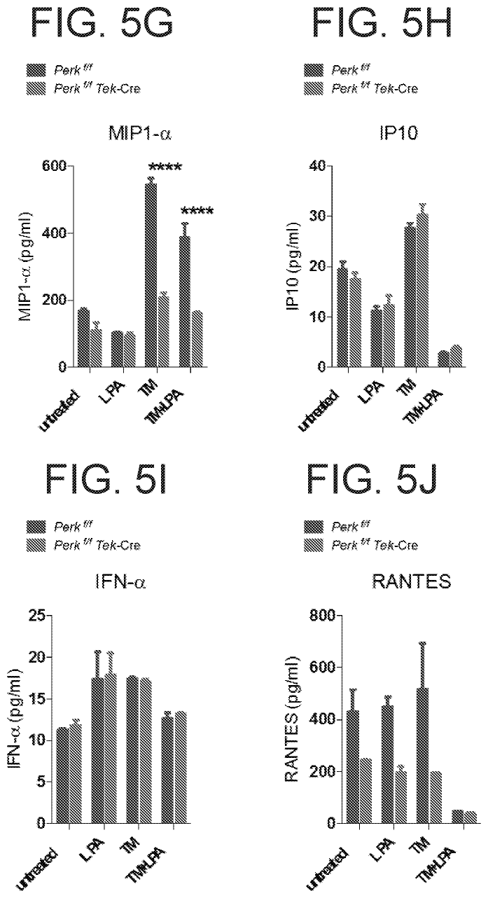

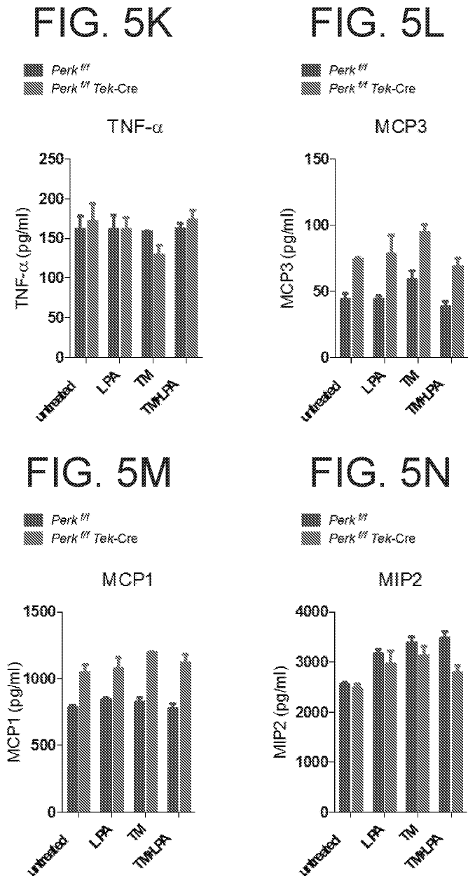

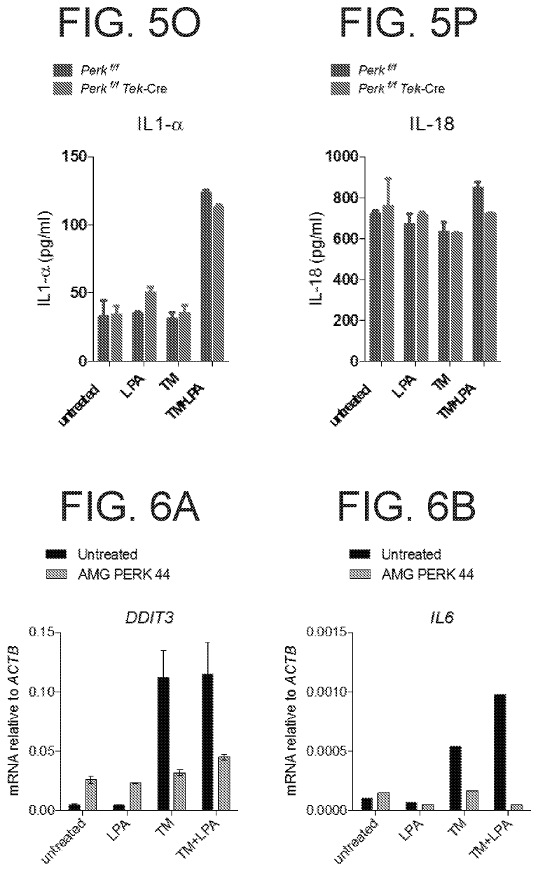

These results were further confirmed at the protein level using Multiplex cytokine assays. As shown in FIG. 5, bone marrow-derived DCs co-treated with the ER stressor Tunicamycin (TM) and physiological concentrations of LPA found in human ovarian cancer ascites (100 mM), demonstrated significant PERK-dependent overproduction of tumorigenic IL-1b, IL-6, VEGF-a, LIF, M-CSF, GRO-a (IL-8) and MIP1-a, while expression of protective anti-tumor cytokines like IFN-a, RANTES (CCL5) and TNF-a remained unaltered.

The findings described above were confirmed by analyzing human primary DCs treated with ER stressors and LPA in the presence or absence of the PERK inhibitor AMG PERK 44 (Tocris), which the inventors had tested and confirmed to recapitulate the effects of PERK deletion in murine DCs in vitro (data not shown). The structure of AMG PERK 44 is shown below as a HCl salt.

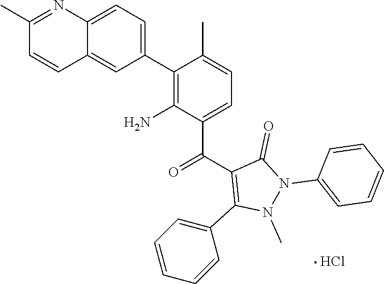

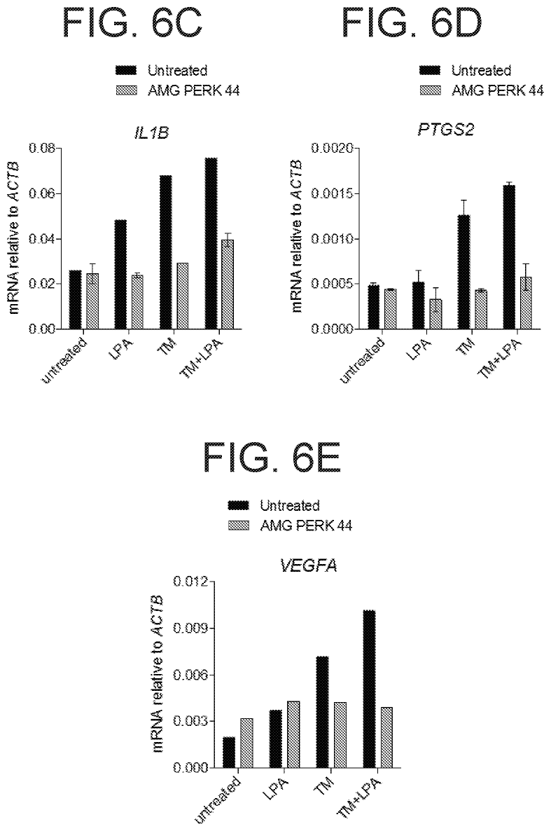

As shown in FIG. 6, human DCs undergoing ER stress and simultaneously exposed to LPA demonstrated robust PERK-dependent induction of IL6, IL1B, PTGS2 and VEGFA.

These results demonstrate, for the first time, that ER stress-activated PERK signaling amplifies the effects of LPA sensing by DCs. These data also indicate that disabling LPA biosynthetic pathways, LPA receptors/sensors or targeting PERK in DCs, could be used for anti-cancer therapeutic purposes.

### Example 4: PERK Deletion in Dendritic Cells Extends Survival in Ovarian Cancer Hosts

This Example shows that ablation of PERK in myeloid dendritic cells can improve survival of subjects that have cancer.

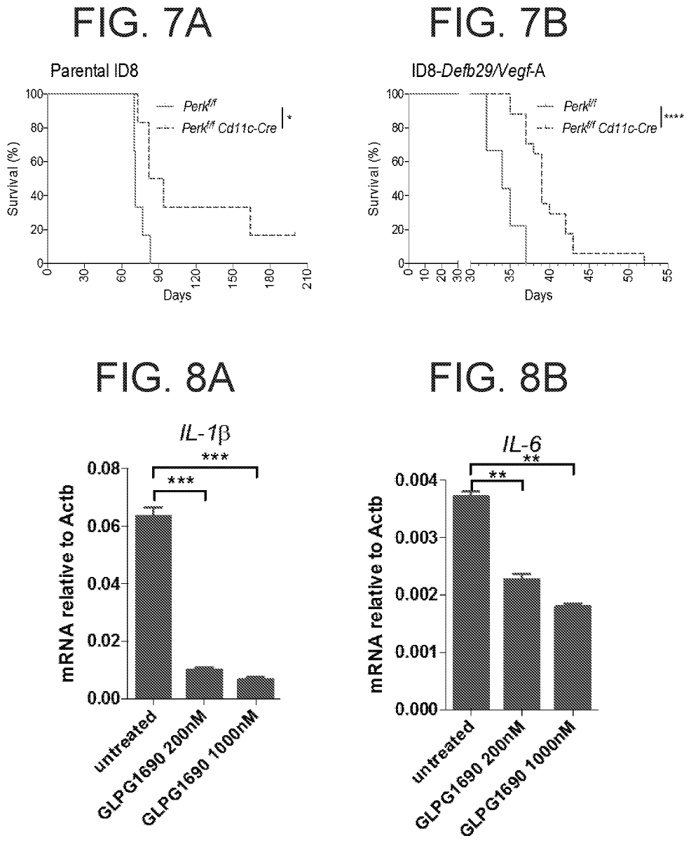

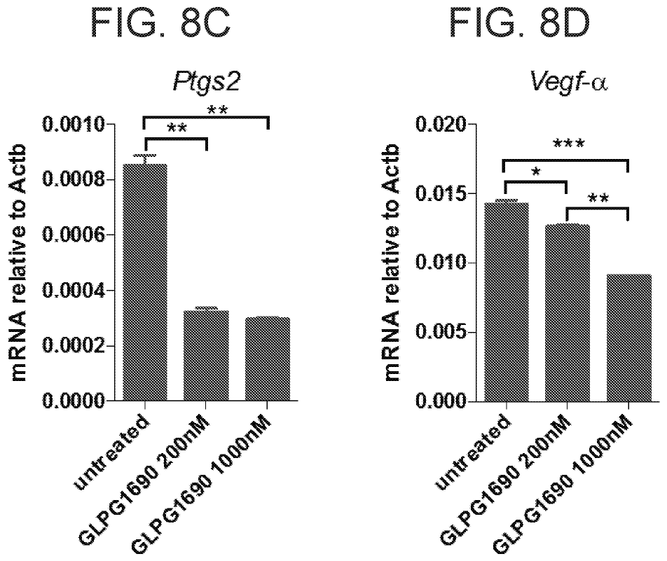

To determine the in vivo relevance of the foregoing findings metastatic ovarian cancer was developed in female mice that selectively lack PERK in CD11c⁺ DCs (Perk^{f/f} Cd11c^{cre}). Strikingly, PERK deficiency in these myeloid cells significantly extended host survival, compared with their wild-type counterparts (FIG. 7) using two independent models of disease. These data show that PERK expression in host DCs is necessary for the aggressive progression of metastatic ovarian cancer.

Further experiments were performed to ascertain whether inhibiting LPA biosynthetic pathways could be used to influence DC functions in the tumor microenvironment. Since Autotaxin is the main enzyme involved in LPA generation, the selective Autotaxin inhibitor GLPG1690 was used for this purpose. The structure of the GLPG1690 molecule is shown below.

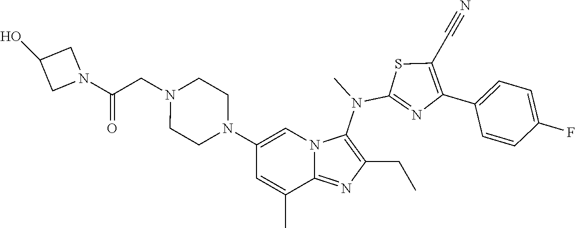 [SMILES: CCc1nc2c(C)cc(N3CCN(CC(=O)N4CC(O)C4)CC3)cn2c1N(C)c1nc(-c2ccc(F)cc2)c(C#N)s1]

Ovarian cancer ascites samples containing multiple immune and malignant cell types were obtained from tumor-bearing mice and incubated ex vivo with GLPG1690, and DCs present in this malignant fluid were isolated by FACS 24 h later. Of note, GLPG1690 treatment decreased the expression of the LPA/ER stress-induced Il1b, Il6, Ptgs2 and Vegf-α in these tumor-associated DCs (FIG. 8).

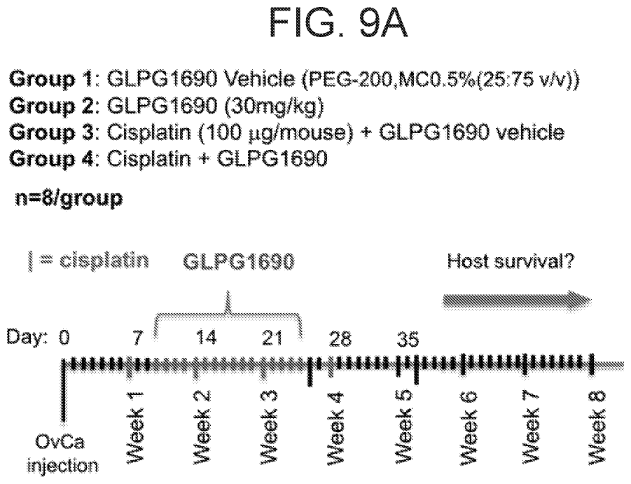

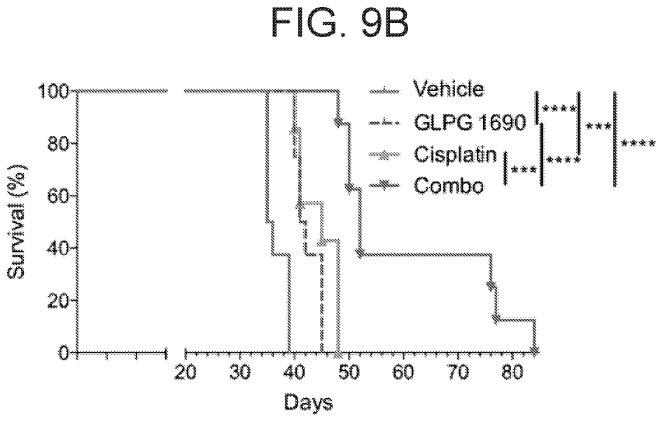

Next, experiments were performed to determine whether treatment with GLPG1690 could induce anti-ovarian cancer effects, an approach that has not been attempted or reported to date. As shown in FIG. 9, targeting LPA generation with this small molecule inhibitor modestly extended host survival when used as a single treatment (FIG. 9). However, GLPG1690 treatment significantly enhanced the effects of chemotherapy in mice bearing metastatic ovarian cancer (FIG. 9).

These results show that a previously unappreciated protumoral network exists in ovarian cancer that is coordinated by the phospholipid messenger LPA and PERK-driven ER stress responses in DCs. These results also show that inhibitors of LPA production enhance the effects of chemotherapy in combating cancers such as metastatic ovarian cancer.

### Example 5: LPA Reduced Expression of Genes Induced by Interferon in Dendritic Cells

This Example illustrates that LPA exposure blocked the expression of genes typically induced by type-I interferon (IFNα/β).

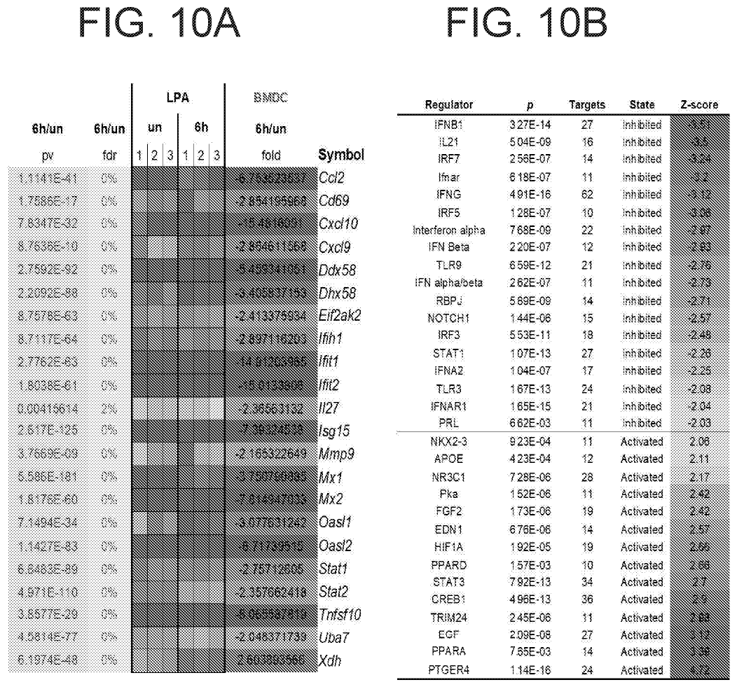

RNA was obtained from LPA-treated bone marrow-derived DCs (BMDCs) and RNA sequencing was performed. The RNA-sequencing data and Ingenuity Pathway Analyses (IPA) was performed on the RNA from LPA-treated bone marrow-derived DCs (BMDCs). These experiments revealed, unexpectedly, that LPA exposure blocked the expression of genes typically induced by type-I interferon (IFNα/β) (FIG. 10).

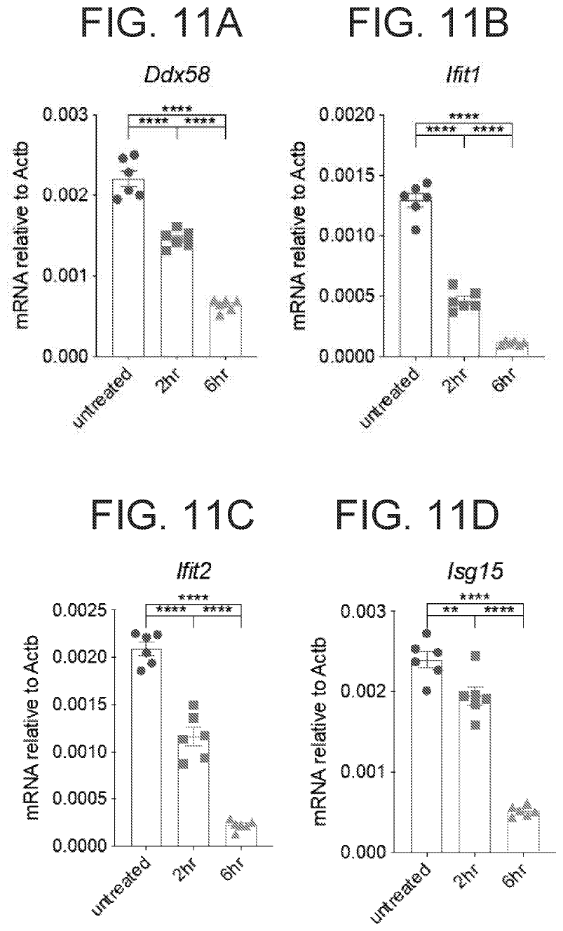

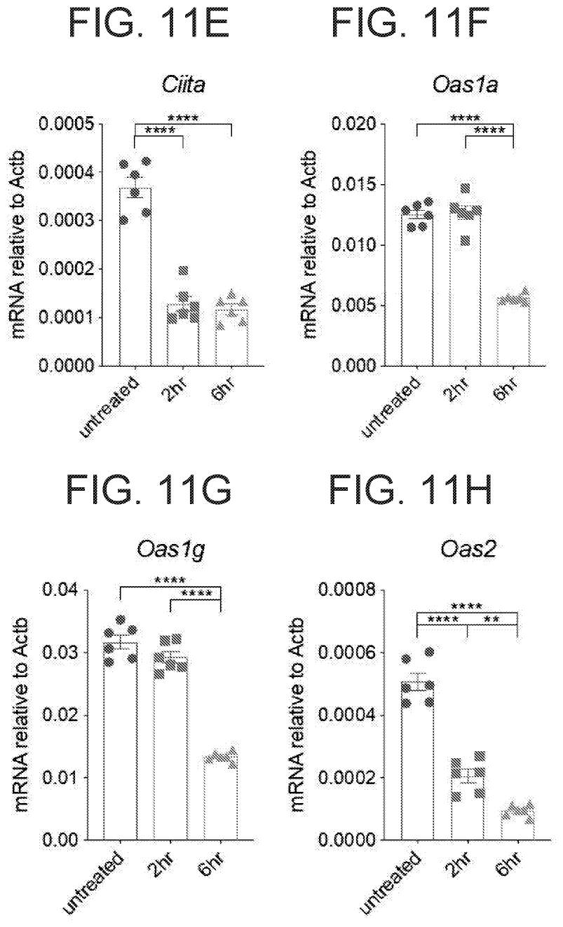

These results were confirmed via RT-qPCR evaluation of type-I IFN target gene expression such as Ddx58, Ifit1, Ifit2, Isg15, Ciita, Oas1a, Oas1g and Oas2, all of which were decreased upon LPA exposure (FIG. 11A-11H).

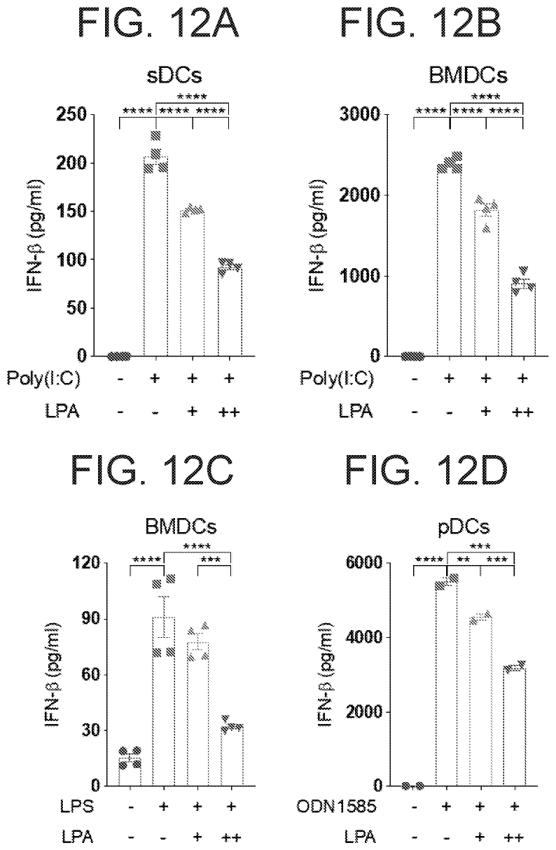

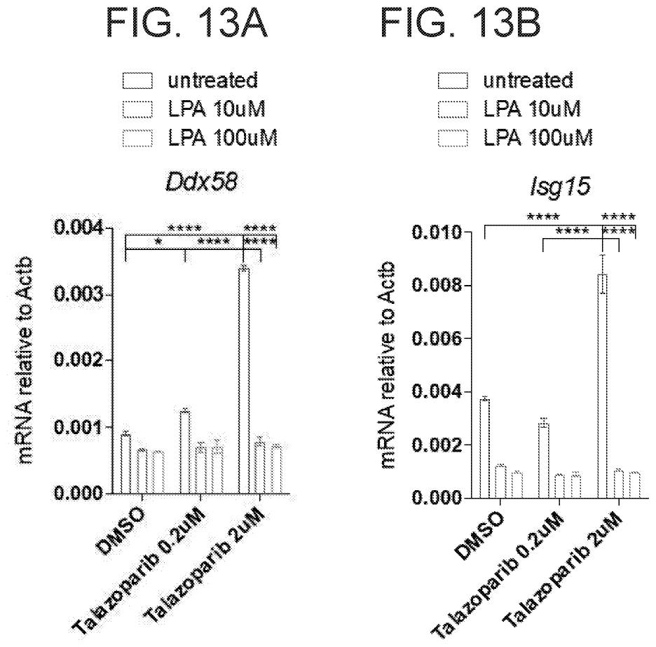

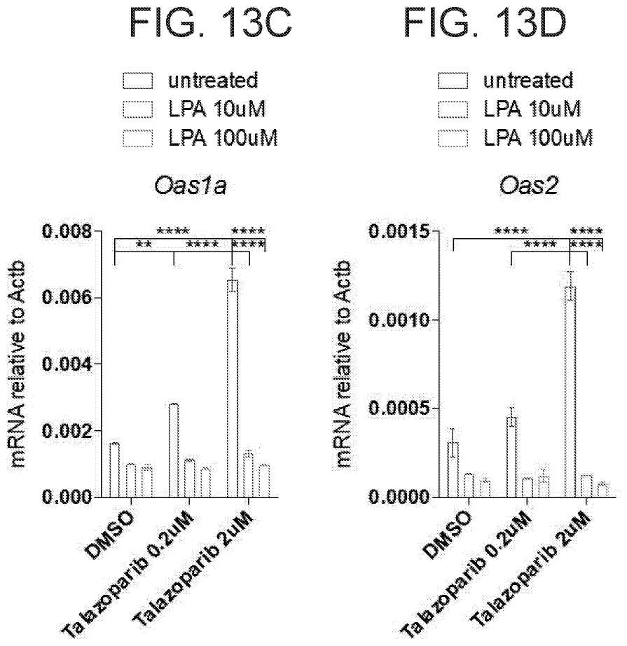

These effects also occurred in diverse DC types such as BMDCs, splenic DCs (sDCs) and plasmacytoid DCs (pDCs) during stimulation through Toll-like Receptors (TLRs) or upon exposure to cancer cells treated with inhibitors of poly ADP-ribose polymerase (PARP) that induce DNA damage. As shown in FIG. 12A-12D, BMDCs, sDCs and pDCs exposed to LPA expressed decreased levels of IFN-β protein upon TLR agonist stimulation. LPA also prevented expression of type-I IFN-related genes in BMDCs exposed to ovarian cancer cells treated with the PARP inhibitor Talazoparib (FIG. 13A-13D). The structure of Talazoparib is shown below.

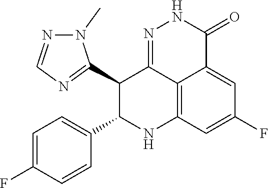 [SMILES: Cn1ncnc1[C@H]1c2n[nH]c(=O)c3cc(F)cc(c23)N[C@@H]1c1ccc(F)cc1]

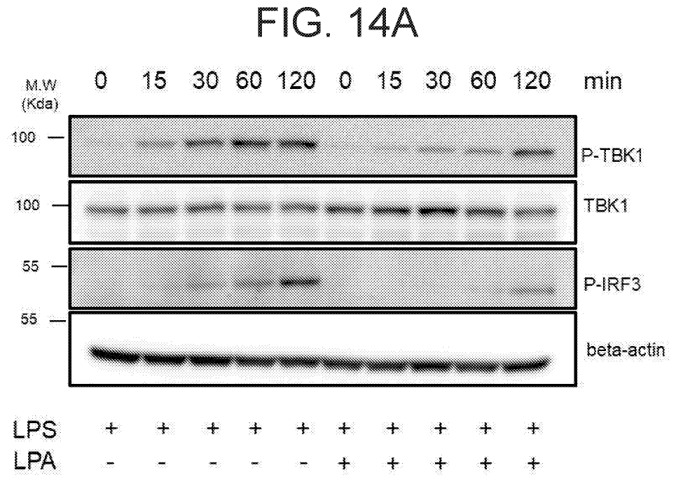

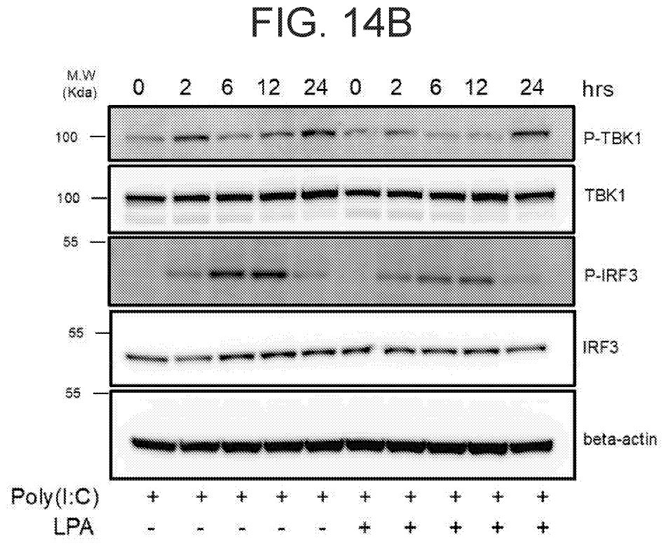

The activation status of signaling pathways implicated in the optimal induction of type-I IFNs was then evaluated. Compared with untreated BMDCs, LPA exposure inhibited phosphorylation of TBK1 and IRF3 proteins in LPS-BMDCs or Poly(I:C)-treated BMDCs (FIG. 14). These data unveil that LPA sensing by DCs abrogates the activation of key signaling mediators, such as TBK1 and IRF3, in order to blunt optimal type-1 IFN expression.

### Example 6: Autotaxin-Deficiency Increases Survival of Animals with Ovarian Cancer

This Example illustrates that inhibition of autotaxin increases survival of animals with ovarian cancer.

To define the in vivo relevance of the findings related to type-I IFN expression, the inventors abrogated the gene encoding the LPA-generating enzyme autotaxin (Enpp2) in ID8-based ovarian cancer cells lines using CRISPR-Cas9.

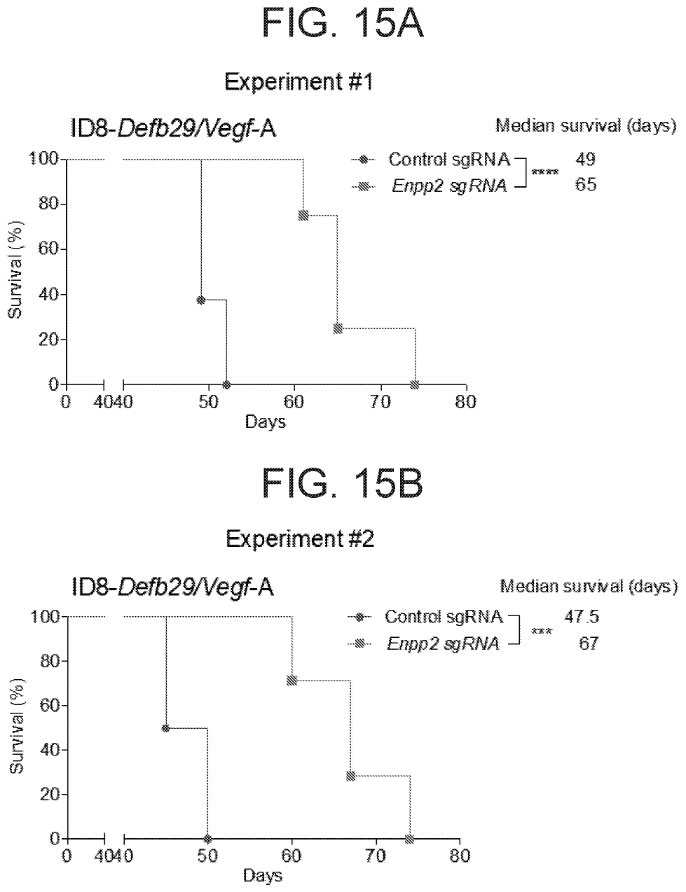

Female mice challenged with autotaxin-deficient ovarian cancer cells demonstrated a remarkable increase in survival compared with littermate controls implanted with isogenic cancer cell lines harboring scrambled sgRNAs that do not target the murine genome (FIG. 15A-15B). These effects were reproducible in two independent experiments using distinct cancer cell clones (FIG. 15). Hence, malignant cells represent a major source of autotaxin in the ovarian cancer microenvironment and these results indicate that the LPA generating autotaxin enzyme is indeed a pro-tumorigenic pathway that promotes disease progression in this malignancy.

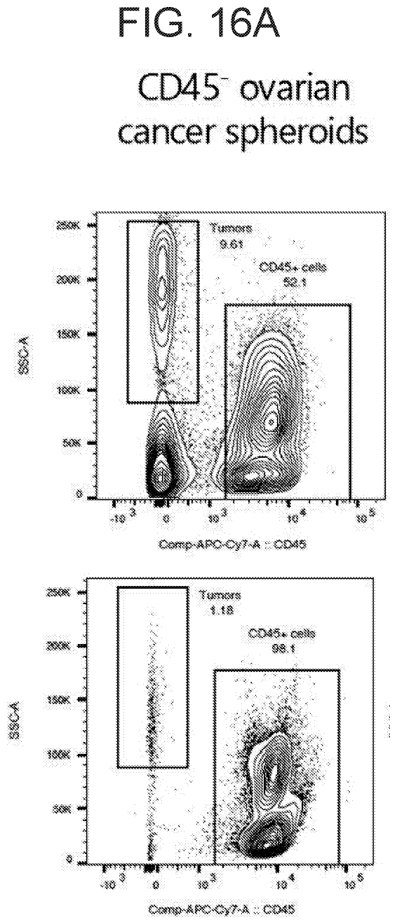

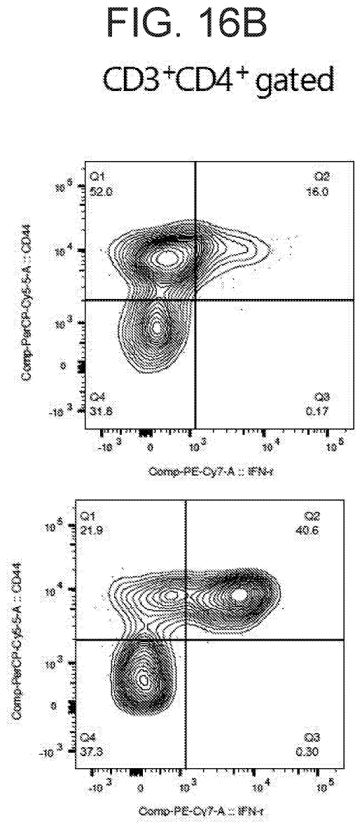

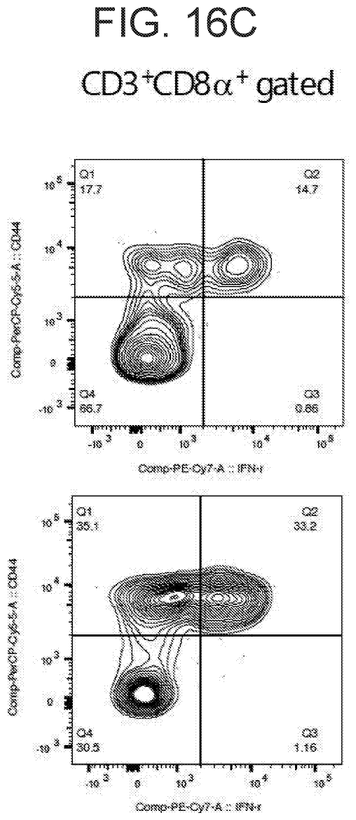

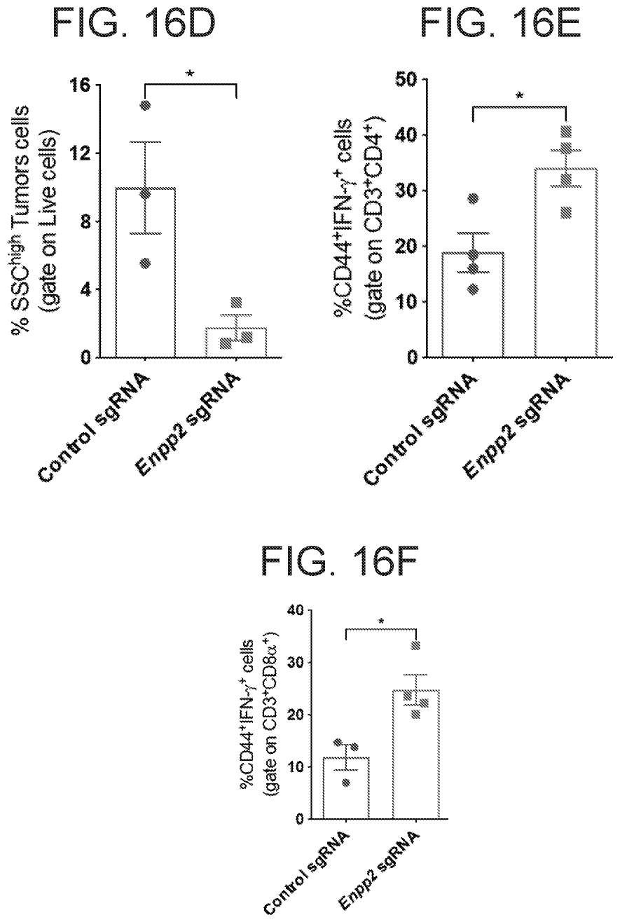

Immunophenotyping experiments were also performed at 4 weeks after tumor inoculation. The results show that loss of autotaxin in the cancer cells correlated with decreased proportions of malignant spheroids in the peritoneal cavity, and with enhanced infiltration by activated T cells producing IFNγ in situ (FIG. 16). These data demonstrate that the LPA-generating autotaxin also operates as an immunosuppressive mediator that inhibits T cell activation in the tumor microenvironment.

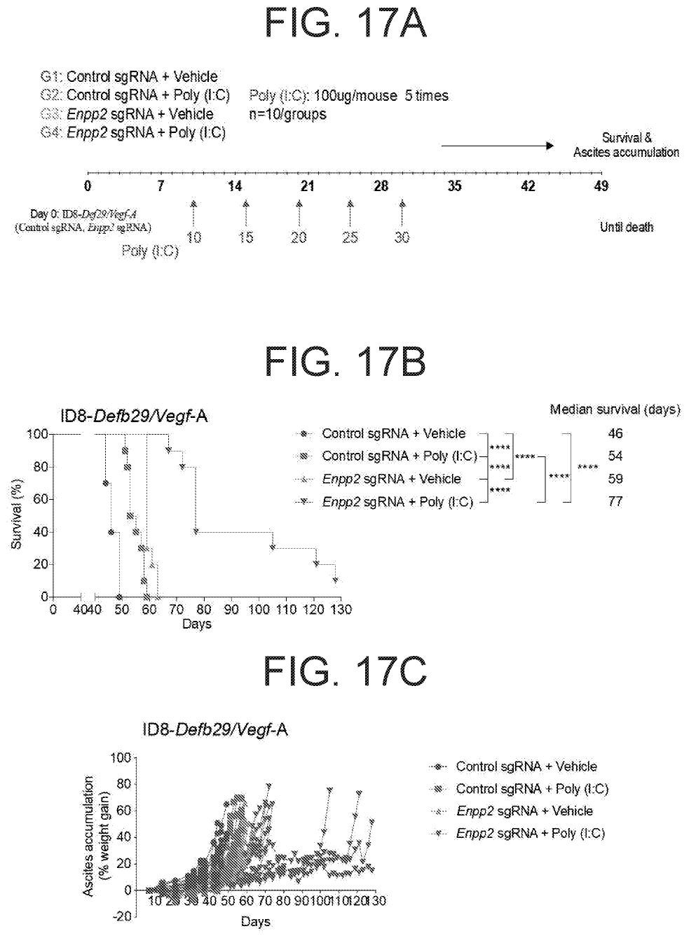

Based on these key findings, experiments were then performed to determine whether treatment with the TLR3 agonist Poly(I:C), which can enhance type-I IFN immune responses, could increase survival in mice bearing autotaxin-deficient ovarian tumors. Confirming our prior results, mice bearing autotaxin-deficient cancer cells demonstrated prolonged survival compared with their littermate controls bearing control sgRNA-transfected ovarian cancer cells (FIG. 17). Poly(I:C) treatment alone also extended host survival (FIG. 17). Strikingly, treatment with Poly(I:C) in mice bearing autotaxin-deficient ovarian cancer showed a remarkable increase in survival compared with all other experimental groups (FIG. 17B).

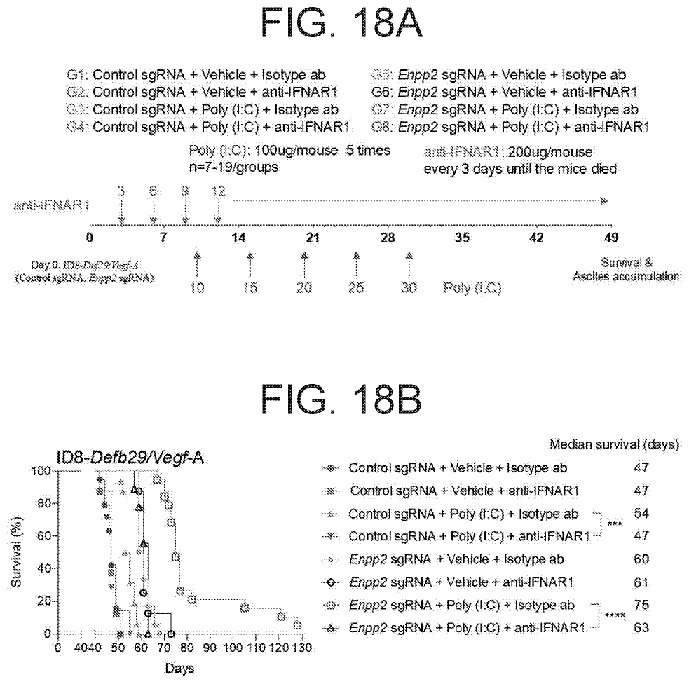

To determine whether these effects are really mediated by enhanced type-I IFN signaling, mice were treated with anti-IFNAR1 blocking antibodies. Blockade of IFNAR1 signaling with this approach fully abrogated the therapeutic effects Poly-(I:C) in mice bearing autotaxin-deficient ovarian tumors (FIG. 18). These data reveal that LPA signaling operates as a negative regulator of type-I IFN expression in the ovarian cancer microenvironment, and that targeting autotaxin-LPA can unleash protective anti-tumor type-I IFN responses.

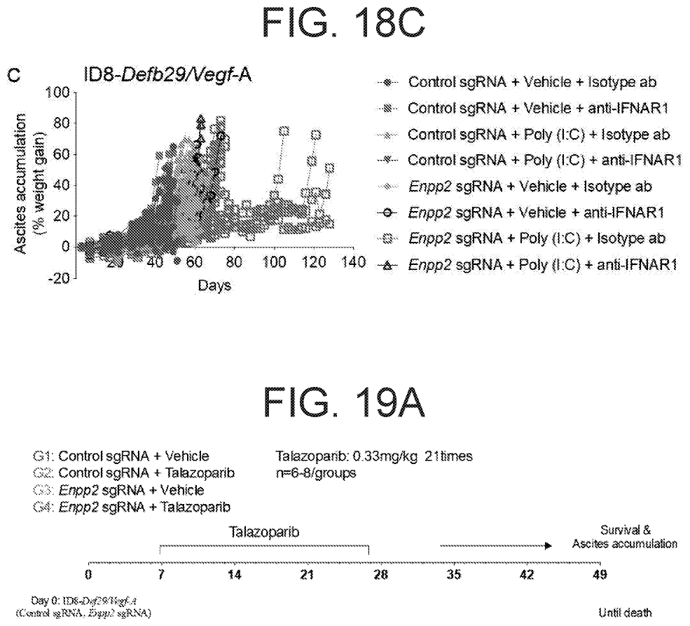

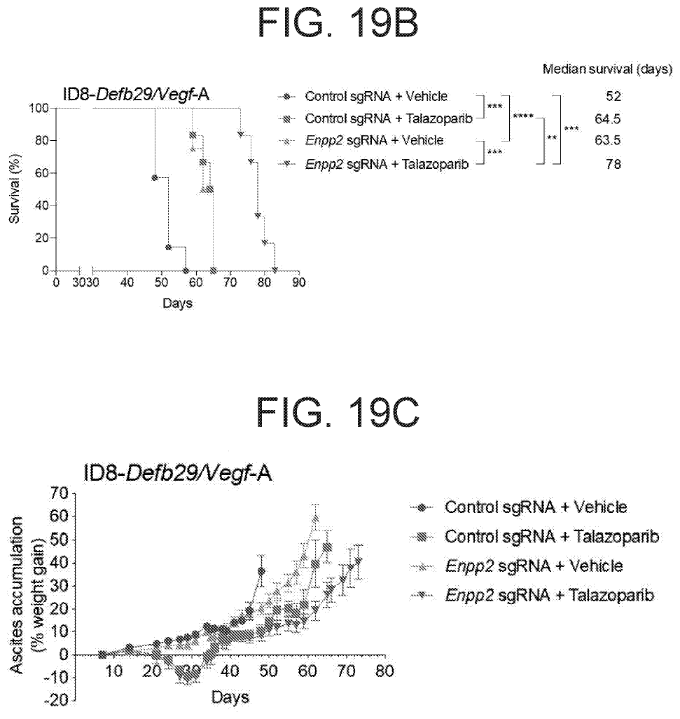

Experiments were also performed to determine whether disabling autotaxin-LPA expression could be used to improve the therapeutic efficacy of other anti-ovarian cancer agents, such as PARP inhibitors, which can activate type-I IFN responses. Surprisingly, treatment of mice bearing autotaxin-deficient ovarian cancer with the PARP inhibitor Talazoparib elicited a significant increase in host survival (FIG. 19).

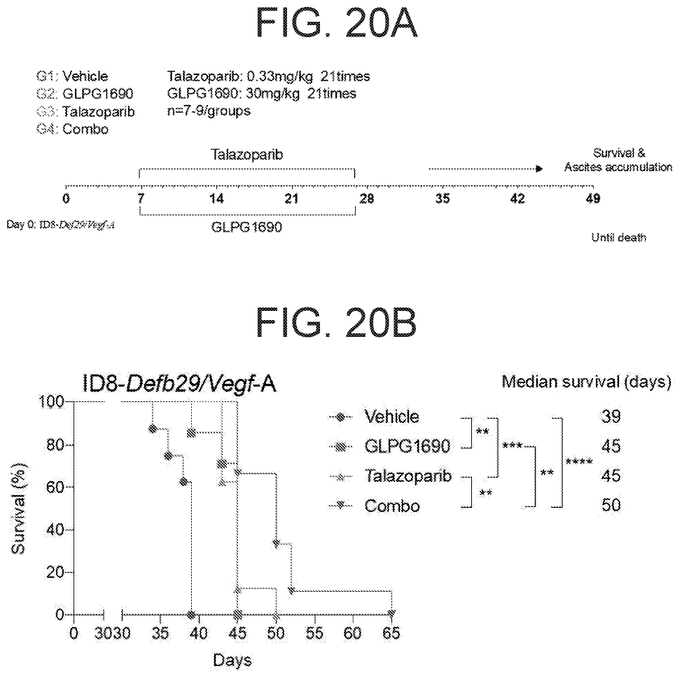

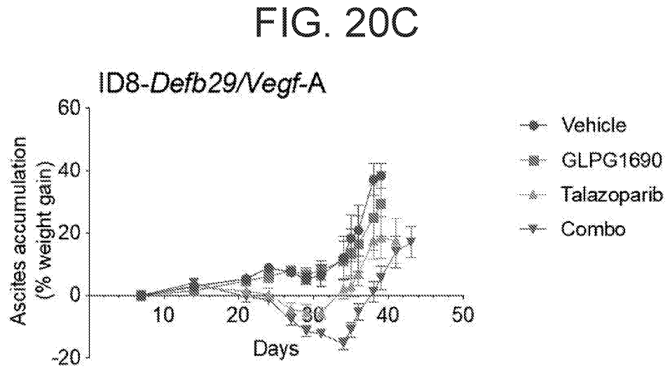

The inventors next determined whether treatment with small-molecule inhibitors for autotaxin (GLPG1690, Galapagos) could induce anti-ovarian cancer effects that improve the efficacy of PARP inhibition, an approach that has not been attempted or reported to date. Targeting LPA generation with this small molecule inhibitor modestly extended host survival when used as a single agent treatment. However, GLPG1690 administration significantly improved the therapeutic effects of Talazoparib in mice bearing metastatic ovarian cancer (FIG. 20). Taken together, these new data indicate that the autotaxin-LPA axis operates as a new immunosuppressive mechanism that promotes ovarian cancer progression by interrupting optimal type-I IFN responses. Consequently, production of this bioactive lipid mediator limits the effects of immunotherapies such as Poly-(I:C) treatment, and of novel chemotherapeutic interventions such as PARP inhibitors. Targeting the autotaxin-LPA pathway may therefore be beneficial to control ovarian cancer progression and unleash protective anti-tumor immune responses that extend host survival. Of particular importance, autotaxin inhibitors such as GLPG1690 are now being tested in the clinic in the setting of pulmonary diseases. Therefore, our findings indicate that autotaxin inhibitors could be repurposed to maximize the efficacy of PARP inhibitors and awaken protective type-I IFN responses in ovarian cancer patients.

### Example 7: GLPG1690 is More Effective than Some Other Autotaxin Inhibitors

This Example illustrates that is more effective than some other autotaxin inhibitors

Methods similar to those described above were used to evaluate the anti-tumor effects of various autotaxin and LPA receptor inhibitors. The inhibitors were administered to mice that had received ovarian cancer cells that overexpress VEGFA and Defb29 (ID8-Defb29/Vegf-A).

As shown in Table 4, only GLPG1690 was able to increase the survival of the mice, administered either as a single agent or when used in combination with other chemotherapeutic agents.

TABLE 4
Anti-Tumor Efficacy of Autotaxin/LPA Receptor Inhibitors

| Inhibitor (Target) | Administration Route | Efficacy |
|---|---|---|
| Ki16425 | I.P. | N.E. |
| (LPAR1, LPAR3) | | |
| Ki16198 | I.P. | N.E. |
| (LPAR1, LPAR3 | | |
| AM095 (LPAR1) | I.P. | N.E. |
| PF8380 (ATX) | I.P. | N.E. |
| GLPG1690 (ATX) | Oral | Increased survival |
| | | (singly or with other |
| | | agents) |
| ONO-840506 (ATX) | Oral | N.E. |

N.E.: no effect, I.P.: intra peritoneal, ATX: autotaxin, LPAR: LPA receptor

### Example 8: Abrogation of PERK and Autotaxin Increases Mammalian Survival

This Example illustrates that concomitant abrogation of ER stress sensor PERK in dendritic cells (DCs) and autotaxin in ovarian cancer cells elicits a synergistic increase in host survival.

Female mice that selectively lack PERK in CD11c⁺ DCs (Eif2ak3^{f/f} Cd11c-Cre), or their littermate controls (Eif2ak3^{f/f}), were challenged with ID8-based ovarian tumors devoid of autotaxin (Enpp2 sgRNA), or with their corresponding isogenic controls harboring scrambled sgRNA (Control sgRNA).

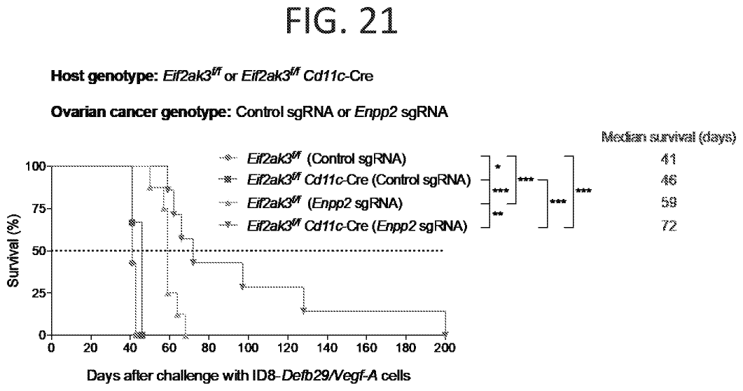

FIG. 21 shows that maximal survival occurs in mice with dendritic cells that are deficient in PERK when autotaxin-ablated ovarian tumors are present. Hence, the PERK-autotaxin pathway is a key enhancer of metastatic ovarian cancer progression. Inhibiting or ablating PERK in dendritic cells while also inhibiting autotaxin (especially in cancer cells) can effectively treat cancer.

### REFERENCES

1. Fang X, Gaudette D, Furui T. Mao M, Estrella V. Eder A, et al. Lysophospholipid growth factors in the initiation, progression, metastases, and management of ovarian cancer. Ann N Y Acad Sci. 2000; 905:188-208.
2. Fang X, Schummer M, Mao M. Yu S, Tabassam F H, Swaby R, et al. Lysophosphatidic acid is a bioactive mediator in ovarian cancer. Biochimica et biophysica acta. 2002; 1582(1-3):257-64.
3. Hu Y L, Albanese C, Pestell R G, and Jaffe R B. Dual mechanisms for lysophosphatidic acid stimulation of human ovarian carcinoma cells. J Natl Cancer Inst. 2003; 95(10):733-40.
4. Yamada T. Sato K, Komachi M, Malchinkhuu E, Tobo M, Kimura T, et al. Lysophosphatidic acid (LPA) in malignant ascites stimulates motility of human pancreatic cancer cells through LPA1. J Biol Chem. 2004; 279(8):6595-605.
5. Panupinthu N. Lee H Y, and Mills G B. Lysophosphatidic acid production and action: critical new players in breast cancer initiation and progression. Br J Cancer. 2010; 102(6):941-6.
6. Murph M M, Liu W, Yu S, Lu Y, Hall H, Hennessy B T, et al. Lysophosphatidic acid-induced transcriptional profile represents serous epithelial ovarian carcinoma and worsened prognosis. PLoS One. 2009; 4(5):e5583.
7. Cubillos-Ruiz J R, Silberman P C, Rutkowski M R, Chopra S, Perales-Puchalt A, Song M, et al. E R Stress Sensor XBP1 Controls Anti-tumor Immunity by Disrupting Dendritic Cell Homeostasis. Cell. 2015; 161(7):1527-38.
8. Cubillos-Ruiz J R, Bettigole S E, and Glimcher L H. Molecular Pathways: Immunosuppressive Roles of IRE1alpha-XBP1 Signaling in Dendritic Cells of the Tumor Microenvironment. Clin Cancer Res. 2016; 22(9):2121-6.
9. Cubillos-Ruiz J R, Bettigole S E, and Glimcher L H. Tumorigenic and Immunosuppressive Effects of Endoplasmic Reticulum Stress in Cancer. Cell. 2017; 168(4):692-706.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following Statements summarize aspects and features of the invention.

### Statements:

(1) A composition comprising one or more inhibitors of: (a) lysophosphatidic acid (LPA) production, (b) LPA receptor(s), (c) PERK expression or PERK activation, or (d) a combination of such inhibitors in an amount effective for increasing interferon in dendritic cells within a mammalian subject.
(2) The composition of statement 1, which reduces lysophosphatidic acid (LPA) production or LPA signaling by at least 10%, or at least 20%, or at least 30%, or at least 40%, or at least 50%, or at least 60%, or at least 70%, or at least 80%, or at least 90%, or at least 95% in the dendritic cells compared to control dendritic cells untreated by the one or more inhibitors.
(3) The composition of statement 1 or 2, which reduces expression of at least one of PERK, eif2ak3, IL6, IL1B, PTGS2, Enpp2, or VEGFA by at least 10%, or at least 20%, or at least 30%, or at least 40%, or at least 50%, or least 60%, or at least 70%, or at least 80%, or at least 90%, or at least 95% in the dendritic cells compared to control dendritic cells untreated by the one or more inhibitors.
(4) The composition of statement 1, 2 or 3, which reduces expression Atf4, Ddit3, Asns, or a combination thereof in the subject to which the composition is administered.
(5) The composition of statement 1-3 or 4, which increases expression of Ddx58, Ifit1, Ifit2, Isg15, Ciita, Oas1a, Oas1g, Oas2 or a combination thereof in the subject to which the composition is administered.
(6) The composition 1-4, or 5, which increases interferon in dendritic cells within a mammalian subject by at least 10%, or at least 20%, or at least 30%, or at least 40%, or at least 50%, or least 60%, or at least 70%, or at least 80%, or at least 90%, or at least 95% compared to control dendritic cells untreated by the one or more inhibitors.
(7) The composition 1-5 or 6, which increases interferon in dendritic cells within a mammalian subject by at least 2-fold or at least 3-fold compared to control dendritic cells untreated by the one or more inhibitors.
(8) The composition 1-6 or 7, which increases type 1 interferon signaling within a mammalian subject by at least 2-fold or at least 3-fold compared to control dendritic cells untreated by the one or more inhibitors.
(9) The composition of statement 1-7 or 8, with inhibits enzymatic activity of Autotaxin by at least 10%, or at least 20%, or at least 30%, or at least 40%, or at least 50%, or at least 60%, or at least 70%, or at least 80%, or at least 90%, or at least 95% in dendritic cells or in cancer cells compared to control untreated dendritic cells or control untreated cancer cells.
(10) The composition of statement 1-8 or 9, wherein the inhibitor is one or more of GLPG1690, octanoylglycerol pyrophosphate (DGPP 8.0), 2-[[(E)-octadec-9-enoyl]amino]ethyl dihydrogen phosphate, (S)-phosphoric acid mono-[3-(4-benzyloxy-phenyl)-2-octadec-9-enoylamino-propyl] ester (ammonium salt), Ki16425, 2-(2-(2-aminoacetamido)-3-(2,4-dinitrophenylthio)propanamido)pentanedioic acid (NSC161613). AM152 (chemical name (R)-1-(4′-(3-methyl-4-(((1-phenylethoxy)carbonyl)amino)isoxazol-5-yl)-[1,1′-biphenyl]-4-yl)cyclopropane-1-carboxylic acid). VPC32183 (chemical name [(2R)-2-[[(Z)-Octadec-9-enoyl]amino]-3-[4-(pyridin-3-ylmethoxy)phenyl]propyl]dihydrogen phosphate), VPC12249 ((S)-phosphoric acid mono-[3-(4-benzyloxy-phenyl)-2-octadec-9-enoylamino-propyl] ester), H2L 5765834 (chemical name 2-[3-(4-nitrophenoxy)phenyl]-1,3-dioxoisoindole-5-carboxylic acid), NSC12404 (chemical name 2-[(9-Oxo-9H-fluoren-2-yl)carbamoyl]benzoic acid), GRI977143 (chemical name 2-[[3-(1,3-Dioxo-1H-benz[de]isoquinolin-2(3H)-yl)propyl]thio]-benzoic acid), H2L5547924 (chemical name 4,5-dichloro-2-((9-oxo-9H-fluoren-2-yl)carbamoyl)benzoic acid), H2L5828102 (chemical name 2-((9,10-dioxo-9,10-dihydroanthracen-2-yl)carbamoyl) benzoic acid), H2L5186303 (chemical name (Z,Z)-4,4′-[1,3-Phenylenebis(oxy-4,1-phenyleneimino)]bis[4-oxo-2-butenoic acid), compound 5987411 (chemical name 2-({3-[(3-propoxybenzoyl)amino]-benzoyl}amino)benzoic acid), AM966, AM095, PF-8380, SAR 100842, compound 35, SBJ-Cpd1, PAT-505, PAT-048, GWJ-A-23 (chemical name [4-(decanoylamino)benzyl]phosphonic acid)), GK442, BMP22 (chemical name (bis(monoacylglycerol)phosphate)), PharmAkea-Cpd A-E, aptamer RB014, BrP-LPA, an autotaxin inhibitor/LPA inhibitor with the following structure, where X is halogen (e.g., Br) and R is C15-C17 alkyl.
(11) The composition of statement 1-9 or 10, wherein the inhibitor is one or more of GSK2606414, GSK2656157, AMG52, AMG PERK 44, or a combination thereof
(12) The composition of statement 1-10 or 11, comprising AMG PERK 44, GLPG1690, Talazoparib, or a combination thereof.
(13) The composition of statement 1-11 or 12, further comprising a second therapeutic agent and/or chemotherapeutic agent selected from one or more PARP inhibitors, alkylating agents (such as nitrogen mustards, alkyl sulfonates, nitrosoureas, ethylenimines, and triazenes); antimetabolites (such as folate antagonists, purine analogues, and pyrimidine analogues); antibiotics (such as anthracyclines, bleomycins, mitomycin, dactinomycin, and plicamycin); enzymes (such as L-asparaginase); farnesyl-protein transferase inhibitors; hormonal agents (such as glucocorticoids, estrogens/antiestrogens, androgens/antiandrogens, progestins, and luteinizing hormone-releasing hormone anatgonists); octreotide acetate; microtubule-disruptor agents (such as ecteinascidins or their analogs and derivatives; microtubule-stabilizing agents (such as paclitaxel (Taxol®), docetaxel (Taxotere®), and epothilones A-F or their analogs or derivatives); plant-derived products (such as vinca alkaloids, epipodophyllotoxins, taxanes); topoisomerase inhibitors; prenyl-protein transferase inhibitors; hydroxyurea; procarbazine; mitotane; hexamethylmelamine; platinum coordination complexes (such as cisplatin and carboplatin); biological response modifiers; growth factors; immune modulators; monoclonal antibodies; or a combination thereof.
(14) The composition of statement 1-12 or 13, which reduces the progression of cancer in the mammalian subject.
(15) The composition of statement 1-13 or 14, which prolongs the survival of the mammalian subject compared to an untreated control.
(16) A method comprising administering the composition of statement 1-14 or 15 to a subject.
(17) A method comprising: a) obtaining dendritic cells from a subject, b) deleting at least a portion of an endogenous PERK (eif2ak3) gene, an Enpp2 gene, or one or more LPAR-encoding genes in one or more dendritic cells to generate PERK-defective, Enpp2-defective, or LPAR-defective dendritic cells; and c) administering a population of the PERK-defective, Enpp2-defective, or LPAR-defective dendritic cells to the subject.
(18) The method of statement 17, further comprising administering the composition of statement 1-8 or 9 to the subject.
(19) The method of statement 16, 17, or 18, which reduces lysophosphatidic acid (LPA) production or LPA signaling by at least 10%, or at least 20%, or at least 30%, or at least 40%, or at least 50%, or at least 60%, or at least 70%, or at least 80%, or at least 90%, or at least 95% in the dendritic cells compared to control dendritic cells untreated by the one or more inhibitors.
(20) The method of statement 16-18 or 19, which reduces expression of at least one of PERK (eif2ak3), IL6, IL1B, PTGS2, Enpp2, or VEGFA by at least 10%, or at least 20%, or at least 30%, or at least 40%, or at least 50%, or least 60%, or at least 70%, or at least 80%, or at least 90%, or at least 95% in the dendritic cells compared to control dendritic cells untreated by the one or more inhibitors.
(21) The method of statement 16-19 or 20, which reduces expression Atf4, Ddit3, Asns, or a combination thereof in the subject to which the composition is administered.
(22) The method of statement 16-20 or 21, which increases expression of Ddx58, Ifit1, Ifit2, Isg15, Ciita, Oas1a, Oas1g, Oas2 or a combination thereof in the subject to which the composition is administered.
(23) The method of statement 16-21 or 22, which increases interferon in dendritic cells within a mammalian subject by at least 10%, or at least 20%, or at least 30%, or at least 40%, or at least 50%, or least 60%, or at least 70%, or at least 80%, or at least 90%, or at least 95% compared to control dendritic cells untreated by the one or more inhibitors.
(24) The method of statement 16-22 or 23, which increases interferon in dendritic cells within a mammalian subject by at least 2-fold or at least 3-fold compared to control dendritic cells untreated by the one or more inhibitors.
(25) The method of statement 16-23 or 24, wherein the subject is suspected of having cancer.
(26) The method of statement 16-24, or 25, wherein the subject has breast cancer, colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, pancreatic cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer.
(27) The method of statement 16-25, or 26, wherein the subject has ovarian cancer, pancreatic cancer, breast cancer (e.g., triple-negative breast cancer), or prostate cancer.
(28) The method of statement 16-26, or 27, wherein the inhibitor is one or more of GLPG1690, octanoylglycerol pyrophosphate (DGPP 8.0), 2-[[(E)-octadec-9-enoyl]amino]ethyl dihydrogen phosphate, (S)-phosphoric acid mono-[3-(4-benzyloxy-phenyl)-2-octadec-9-enoylamino-propyl] ester (ammonium salt), Ki16425, 2-(2-(2-aminoacetamido)-3-(2,4-dinitrophenylthio)propanamido)pentanedioic acid (NSC161613), AM152 (chemical name (R)-1-(4′-(3-methyl-4-(((1-phenylethoxy)carbonyl)amino)isoxazol-5-yl)-[1,1′-biphenyl]-4-yl)cyclopropane-1-carboxylic acid), VPC32183 (chemical name [(2R)-2-[[(Z)-Octadec-9-enoyl]amino]-3-[4-(pyridin-3-ylmethoxy)phenyl]propyl]dihydrogen phosphate), VPC12249 ((S)-phosphoric acid mono-[3-(4-benzyloxy-phenyl)-2-octadec-9-enoylamino-propyl] ester), H2L 5765834 (chemical name 2-[3-(4-nitrophenoxy)phenyl]-1,3-dioxoisoindole-5-carboxylic acid), NSC12404 (chemical name 2-[(9-Oxo-9H-fluoren-2-yl)carbamoyl]benzoic acid), GR1977143 (chemical name 2-[[3-(1,3-Dioxo-1H-benz[de]isoquinolin-2(3H)-yl)propyl]thio]-benzoic acid), H2L5547924 (chemical name 4,5-dichloro-2-((9-oxo-9H-fluoren-2-yl)carbamoyl)benzoic acid), H2L5828102 (chemical name 2-((9,10-dioxo-9,10-dihydroanthracen-2-yl)carbamoyl) benzoic acid), H2L5186303 (chemical name (Z,Z)-4,4′-[1,3-Phenylenebis(oxy-4,1-phenyleneimino)]bis[4-oxo-2-butenoic acid), compound 5987411 (chemical name 2-({3-[(3-propoxybenzoyl)amino]-benzoyl}amino)benzoic acid), AM966, AM095. PF-8380, SAR 100842, compound 35, SBJ-Cpd1, PAT-505, PAT-048, GWJ-A-23 (chemical name 14-(decanoylamino)benzyl]phosphonic acid)), GK442, BMP22 (chemical name (bis(monoacylglycerol)phosphate)), PharmAkea-Cpd A-E, aptamer RB014, BrP-LPA, an autotaxin inhibitor/LPA inhibitor with the following structure, where X is halogen (e.g., Br) and R is C15-C17 alkyl.
(29) The method of statement 16-27, or 28, wherein the inhibitor is one or more of GSK2606414, GSK2656157, AMG52. AMG PERK 44, or a combination thereof.
(30) The method of statement 16-28 or 29, further comprising administering a second therapeutic agent and/or chemotherapeutic agent.
(31) The method of statement 16-29 or 30, further comprising administering a second therapeutic agent and/or chemotherapeutic agent at the same time as administering the population of the PERK-defective, Enpp2-defective, or LPAR-defective dendritic cells to the subject.
(32) The method of statement 16-30 or 31, further comprising administering a second therapeutic agent and/or chemotherapeutic agent before or after administering the population of the PERK-defective, Enpp2-defective, or LPAR-defective dendritic cells to the subject.
(33) The method of statement 16-31 or 32, further comprising administering a second therapeutic agent and/or chemotherapeutic agent selected from one or more PARP inhibitors, alkylating agents (such as nitrogen mustards, alkyl sulfonates, nitrosoureas, ethylenimines, and triazenes); antimetabolites (such as folate antagonists, purine analogues, and pyrimidine analogues); antibiotics (such as anthracyclines, bleomycins, mitomycin, dactinomycin, and plicamycin); enzymes (such as L-asparaginase); farnesyl-protein transferase inhibitors; hormonal agents (such as glucocorticoids, estrogens/antiestrogens, androgens/antiandrogens, progestins, and luteinizing hormone-releasing hormone anatgonists); octreotide acetate; microtubule-disruptor agents (such as ecteinascidins or their analogs and derivatives; microtubule-stabilizing agents (such as paclitaxel (Taxol®), docetaxel (Taxotere®), and epothilones A-F or their analogs or derivatives); plant-derived products (such as vinca alkaloids, epipodophyllotoxins, taxanes); topoisomerase inhibitors; prenyl-protein transferase inhibitors; hydroxyurea; procarbazine; mitotane; hexamethylmelamine; platinum coordination complexes (such as cisplatin and carboplatin); biological response modifiers; growth factors; immune modulators; monoclonal antibodies; or a combination thereof.
(34) The method of statement 16-32 or 33, further comprising administering Talazoparib.
(35) The method of statement 16-33 or 34, further comprising radiation therapy.
(36) The method of statement 16-34 or 35, which improves the survival of the subject by at least 1 day, or at least 2 days, or at least 3 days, or at least 4 days, or at least 5 days, or at least 10 days, or at least 15 days, or at least 20 days, or at least 30 days, or at least 45 days, or at least 60 days, compared to a subject that did not receive the composition.

(37) The method of statement 17-35 or 36, wherein deleting at least a 50 portion of an endogenous PERK (eif2ak3) gene, an Enpp2 gene, or one or more LPAR-encoding genes is by CRISPR modification (e.g., deletion) of at least a portion of an endogenous PERK (eif2ak3) gene, an Enpp2 gene, or one or more LPAR-encoding genes.
(38) A method comprising administering one or more inhibitors of lysophosphatidic acid (LPA) production, (b) LPA receptor(s), (c) PERK activation, or (d) a combination of such inhibitors in an amount effective for increasing interferon.
(39) A method comprising administering a composition having AMG PERK 44, GLPG1690, or a combination thereof, to a subject suspected of having cancer, to thereby improve the survival of the subject by at least 5 days.
(40) Use of the composition of any of statements 1-15 to increase interferon in dendritic cells of a mammalian subject.
(41) Use of the composition of any of statements 1-15 to treat cancer in a mammalian subject.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “an inhibitor” or “a molecule” or “a cell” includes a plurality of such inhibitors, molecules or cells, and so forth. In this document, the term “or” is used to refer to a nonexclusive or, such that “A or B” includes “A but not B,” “B but not A.” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

1. A composition comprising one or more inhibitors of: (a) lysophosphatidic acid (LPA) production, (b) LPA receptor(s), (c) PERK activation, or (d) a combination of such inhibitors in an amount effective for increasing type-I interferon expression in dendritic cells within a mammalian subject.

2. The composition of claim 1, which reduces lysophosphatidic acid (LPA) production or LPA signaling by at least 10%, or at least 20%, or at least 30%, or at least 40%, or at least 50%, or at least 60%, or at least 70%, or at least 80%, or at least 90%, or at least 95% in the dendritic cells compared to control dendritic cells untreated by the one or more inhibitors.

3. The composition of claim 1, which reduces expression of at least one of PERK, IL6, IL1B, PTGS2, Enpp2, or VEGFA by at least 10%, or at least 20%, or at least 30%, or at least 40%, or at least 50%, or least 60%, or at least 70%, or at least 80%, or at least 90%, or at least 95% in the dendritic cells compared to control dendritic cells untreated by the one or more inhibitors.

4. The composition of claim 1, which reduces expression Atf4, Ddit3, Asns, or a combination thereof in the subject to which the composition is administered.

5. The composition of claim 1, which increases expression of Ddx58, Ifit1, Ifit2, Isg15, Ciita, Oas1a, Oas1g, Oas2 or a combination thereof in the subject to which the composition is administered.

6. The composition of claim 1, which increases type-I interferons in dendritic cells within a mammalian subject by at least 10%, or at least 20%, or at least 30%, or at least 40%, or at least 50%, or least 60%, or at least 70%, or at least 80%, or at least 90%, or at least 95% compared to control dendritic cells untreated by the one or more inhibitors.

7. The composition of claim 1, which increases type-I interferons in dendritic cells within a mammalian subject by at least 2-fold or at least 3-fold compared to control dendritic cells untreated by the one or more inhibitors.

8. The composition of claim 1, with inhibits enzymatic activity of Autotaxin by at least 10%, or at least 20%, or at least 30%, or at least 40%, or at least 50%, or at least 60%, or at least 70%, or at least 80%, or at least 90%, or at least 95% in dendritic cells or in cancer cells compared to control untreated dendritic cells or control untreated cancer cells.

9. The composition of claim 1, comprising AMG PERK 44, GLPG1690, Talazoparib, or a combination thereof.

10. The composition of claim 1, further comprising a second therapeutic agent and/or chemotherapeutic agent selected from one or more PARP inhibitors, alkylating agents, antimetabolites, antibiotics, L-asparaginases, farnesyl-protein transferase inhibitors, glucocorticoids, estrogens/antiestrogens, androgens/antiandrogens, progestins, luteinizing hormone-releasing hormone anatgonists, octreotide acetate, microtubule-disruptor agents, microtubule-stabilizing agents, epothilones A-F, vinca alkaloids, epipodophyllotoxins, taxanes, topoisomerase inhibitors, prenyl-protein transferase inhibitors, hydroxyurea, procarbazine, mitotane, hexamethylmelamine, platinum coordination complexes, growth factors, immune modulators, monoclonal antibodies, or a combination thereof.

11. The composition of claim 1, which reduces the progression of cancer in the mammalian subject.

12. The composition of claim 1, which prolongs the survival of the mammalian subject.

13. A method comprising administering the composition of claim 1 to a subject.

14. A method comprising: a) obtaining dendritic cells from a subject, b) deleting at least a portion of an endogenous PERK gene, an Enpp2 gene, or one or more LPAR-encoding genes in one or more dendritic cells to generate PERK-defective, Enpp2-defective, or LPAR-defective dendritic cells; and c) administering a population of the PERK-defective, Enpp2-defective, or LPAR-defective dendritic cells to the subject.

15. The method of claim 14, further comprising administering the composition of claim 1-8 or 9 to the subject.

16. The method of claim 14, which reduces lysophosphatidic acid (LPA) production or LPA signaling by at least 10%, or at least 20%, or at least 30%, or at least 40%, or at least 50%, or at least 60%, or at least 70%, or at least 80%, or at least 90%, or at least 95% in the dendritic cells compared to control dendritic cells untreated by the one or more inhibitors.

17. The method of claim 14, which reduces expression of at least one of PERK, IL6, IL1B, PTGS2, Enpp2, or VEGFA by at least 10%, or at least 20%, or at least 30%, or at least 40%, or at least 50%, or least 60%, or at least 70%, or at least 80%, or at least 90%, or at least 95% in the dendritic cells compared to control dendritic cells untreated by the one or more inhibitors.

18. The method of claim 14, which reduces expression of Atf4, Ddit3, Asns, or a combination thereof in the subject.

19. The method of claim 14, which increases expression of Ddx58, Ifit1, Ifit2, Isg15, Ciita, Oas1a, Oas1g, Oas2 or a combination thereof in the subject to which the composition is administered.

20. The method of claim 14, which increases type-I interferons in dendritic cells within a mammalian subject by at least 10%, or at least 20%, or at least 30%, or at least 40%, or at least 50%, or least 60%, or at least 70%, or at least 80%, or at least 90%, or at least 95% compared to control dendritic cells untreated by the one or more inhibitors.

21. The method of claim 14, which increases type-I interferons in dendritic cells within a mammalian subject by at least 2-fold or at least 3-fold compared to control dendritic cells untreated by the one or more inhibitors.

22. The method of claim 14, wherein the subject is suspected of having cancer.

23. The method of claim 14, wherein the subject has breast cancer, colon cancer, intestinal cancer, leukemia, sarcoma, osteosarcoma, lymphomas, melanoma, glioma, pheochromocytoma, hepatoma, ovarian cancer, skin cancer, testicular cancer, gastric cancer, pancreatic cancer, renal cancer, pancreatic cancer, prostate cancer, colorectal cancer, cancer of head and neck, brain cancer, esophageal cancer, bladder cancer, adrenal cortical cancer, lung cancer, bronchus cancer, endometrial cancer, nasopharyngeal cancer, cervical or liver cancer.

24. The method of claim 14, wherein the subject has ovarian cancer or pancreatic cancer.

25. The method of claim 14, further comprising administering a second therapeutic agent and/or chemotherapeutic agent.

26. The method of claim 14, further comprising administering a second therapeutic agent and/or chemotherapeutic agent at the same time as administering the population of the PERK-defective, Enpp2-defective, or LPAR-defective dendritic cells to the subject.

27. The method of claim 14, further comprising administering a second therapeutic agent and/or chemotherapeutic agent before or after administering the population of the PERK-defective, Enpp2-defective, or LPAR-defective dendritic cells to the subject.

28. The method of claim 14, further comprising administering a second therapeutic agent and/or chemotherapeutic agent selected from one or more PARP inhibitors, alkylating agents, antimetabolites, antibiotics, L-asparaginases, farnesyl-protein transferase inhibitors, glucocorticoids, estrogens/antiestrogens, androgens/antiandrogens, progestins, luteinizing hormone-releasing hormone anatgonists, octreotide acetate, microtubule-disruptor agents, microtubule-stabilizing agents, epothilones A-F, vinca alkaloids, epipodophyllotoxins, taxanes, topoisomerase inhibitors, prenyl-protein transferase inhibitors, hydroxyurea, procarbazine, mitotane, hexamethylmelamine, platinum coordination complexes, growth factors, immune modulators, monoclonal antibodies, or a combination thereof.

29. The method of claim 14, further comprising administering Talazoparib.

30. The method of claim 14, further comprising radiation therapy.

31. The method of claim 14, which improves the survival of the subject by at least 1 day, or at least 2 days, or at least 3 days, or at least 4 days, or at least 5 days, or at least 10 days, or at least 15 days, or at least 20 days, or at least 30 days, or at least 45 days, or at least 60 days, compared to a subject that did not receive the composition.

32. A method comprising administering to a mammalian subject one or more inhibitors of lysophosphatidic acid (LPA) production, (b) LPA receptor(s), (c) PERK activation, or (d) a combination of such inhibitors in an amount effective for increasing type-I interferon expression in dendritic cells of the mammalian subject.

33. A method comprising administering a composition having AMG PERK 44, GLPG1690, or a combination thereof, to a subject suspected of having cancer, to thereby improve the survival of the subject by at least 5 days.
